(19) United States
(12) Patent Application Publication
(10) Pub. No.: US 2021/0387237 A1
(43) Pub. Date: Dec. 16, 2021

(54) Apparatus, System, and Method for Cleaning, Healing, and Tissue Regeneration

(71) Applicant: SLOAN WATER TECHNOLOGY LIMITED, London (GB)

(72) Inventors: Timothy Grant Leighton, Southampton (GB); Craig Dolder, Southampton (GB); George Bareham, Poole (GB); Mengyang Zhu, Southampton (GB); Carl Knox, Portsmouth (GB); Freya Jane Malcher, Southampton (GB); Maryam Malakoutikhah, Chandler's Ford (GB); Kay Herbert, Winthrop, MA (US)

(21) Appl. No.: 17/346,937

(22) Filed: Jun. 14, 2021

Related U.S. Application Data
(60) Provisional application No. 63/044,628, filed on Jun. 26, 2020.
(60) Provisional application No. 63/038,231, filed on Jun. 12, 2020.

(51) Int. Cl. B08B 3/12; A61L 2/025; A61L 2/18

(52) U.S. Cl. CPC B08B 3/12; A61L 2/025; A61L 2202/17; A61L 2202/16; A61L 2/18

(57) ABSTRACT

An apparatus includes a conical body defining a chamber configured to contain a liquid and having a nozzle located a front end of the conical body and configured to discharge a stream of the liquid, a rear wall positioned at a rear of the conical body and at least partially defining the chamber, an inlet in communication with the chamber for introducing the liquid into the chamber, an acoustic transducer positioned at the rear wall, the acoustic transducer configured to generate acoustic energy and to introduce the acoustic energy into the liquid contained in the chamber, and an exhaust system including a vent in communication with the chamber to remove a mixture of liquid and gas bubbles from the chamber, an exhaust leading away from the vent, and an outlet in communication with the exhaust and configured to discharge the liquid and gas bubbles into or adjacent to the stream.

### CROSS-REFERENCE TO RELATED APPLICATIONS

This application is a non-provisional of, and claims priority to, U.S. Provisional Application No. 63/038,231, filed Jun. 12, 2020, and U.S. Provisional Application No. 63/044,628, filed Jun. 26, 2020, which prior applications are incorporated by reference herein in their entireties.

### FIELD OF THE INVENTION

This disclosure relates to an apparatus for cleaning and/or providing other treatments using ultrasonic techniques, and more specifically, to an apparatus using acoustic energy propagated through a stream of liquid and to methods for cleaning and providing other treatments using such an apparatus.

### BACKGROUND

Cleaning is an essential part of many research, commercial, and public service processes, such as healthcare, laboratory work, joining, and manufacturing, including processing and packaging produce and other beverage/food or biological products (e.g., enzymes, living cells, genetic material, forensic samples, etc.), as well as in the defense, clean water, sewage, chemical, and nuclear sectors. Cleaning of many articles is often a complex process: the object to be cleaned may be complicated, with many crevices or chambers that are inaccessible to many cleaning techniques, and potential contaminants may be very hazardous (a good example being the biopsy endoscope). The object to be cleaned may also be delicate (a good example of this being salad and vegetable matter, electronic microchips, flesh, forensic material etc.) or tolerate minimal levels of scratching and damage (such as optical lenses, jewelry, prestige watch glasses and faces or prestige car finishes). If cleaning induces scratches, those scratches can harbor subsequent contamination (e.g., dirt, biofilm, etc.) and so make subsequent cleaning more difficult, which further increases the importance of avoiding scratching. Often the time available for cleaning is limited, as there is an imperative to move the object along to the next stage of processing or usage after it is cleaned (either because the number of units available for use is limited—as with an endoscope—or because retardation of through-put cuts profile—as, for example, in the production and packaging of fresh produce like salad).

Cleaning often uses relatively large amounts of water, even for “natural” products: the production of 1 ton of wool currently requires use of around 500 tons of water. When one considers the biohazardous waste of a hospital or abattoir, or the cleaning associated with chemical and nuclear plants, water conservation becomes a very major concern. The requirement for thorough cleaning is often in conflict with the requirements not to damage the target to be cleaned or decontaminated, not to use excessive water, not to contaminate the environment with run-off containing chemicals (e.g. cleaning products) or containing removed contaminants (which might then enter groundwater supplies or be difficult to process back into clean water), and not to use excessive energy or manpower or time. Damage to the target may include, for example, degrading food or skin through the use of cleaning or disinfectant products, alcohol gel, etc., and/or excessive heating; scratching surgical instruments or optical or electronic components through brushing or scrubbing, or other damage.

Ultrasonic cleaning has been known in the art for many years, by the use of “ultrasonic cleaning baths,” whereby inertial cavitation and the generation of high speed liquid jets through bubble involution causes the removal of surface contaminants. The exploitation of cavitation in ultrasonic cleaning baths has for decades provided ultrasonic cleaning facilities that are suitable for those applications which have had robust objects to be cleaned (i.e. where cavitation erosion damage of the target is ideally not an issue), where the size of the object to be cleaned is small enough to be immersed in the cleaning bath, and where the cleaning lacks the urgency which would necessitate a portable decontamination or cleaning unit to supply on-the-spot cleaning resulting from, e.g., accidental contamination. In many instances of such cleaning, samples are either cleaned prior to further processing or dispersed within a suitable media as part of a larger methodology. Cleaning or processing is then facilitated by the employment of an ultrasonic bath. This often involves the immersion of a suitable container within the bath. If the target to be cleaned is too large, or too remote from, an immovable cleaning system that requires immersion, cleaning can require the target to be disassembled into smaller components and transported for immersion cleaning. There is therefore particular utility in a cleaning apparatus that can be taken to the target to “clean in place,” reducing the need for disassembling and relocation of the parts. Such immersion systems can also result in re-contamination of the target being cleaned. If such an apparatus operates by a rinse, then the contamination can be flushed away, and optionally can be collected if the contamination is hazardous or environmentally unfriendly, or the collected run-off can be sent for further testing, e.g. by PCR (Polymerase Chain Reaction).

The ultrasonic cleaning action is often attributed to the generation of violent cavitation within the vessel itself and the interaction of these phenomena with the walls of the object in question. This cleaning action is attributed to cavitation events where the inertia of the liquid has had a dominant effect on the bubble dynamics. As one example, such cavitation events may result when a high-speed liquid jet passes through the bubble as a result of involution of the bubble wall and generates a blast wave on impact with liquid or solid. As another example, such cavitation events may result when bubbles collapse with almost spherical symmetry in “transient” or “inertial” cavitation events, generating shock waves in the liquid and highly reactive chemical species such as free radicals. As a further example, such cavitation events may result when clouds of bubbles collapse in a concerted manner to magnify these effects to become greater than would be expected without the cloud effect. Hence the exact mechanism is often associated with “transient cavitation” or more precisely inertial cavitation where the violent collapse phase results in the local generation of extreme conditions such as blast waves, jets, high pressures and associated transient high temperatures, and the generation of free radicals.

However, such ultrasonic cleaning systems may suffer from one or more problems of damage to the target surface, poor cleaning, particularly of three dimensional surfaces (e.g., with crevices), and an inability to clean larger objects or surfaces. Furthermore, the insertion of the object to be cleaned into an ultrasonic cleaning bath may disturb the sound field in a manner which degrades the ability of an ultrasonic cleaning bath to clean the object. Ultrasonic cleaning baths present additional difficulties such as limited size (which limits the maximum size of objects that can be placed within it), lack of portability, and difficulty in processing numerous objects simultaneously (e.g., because one object may shield another, or other parts of itself, from the sound field by acoustic scattering or attenuation) or in a short period of time.

The present disclosure is provided to address these problems and other problems in existing cleaning apparatuses and methods. A full discussion of the features and advantages of the present disclosure is deferred to the following detailed description, which proceeds with reference to the accompanying drawings.

### BRIEF SUMMARY

Aspects of the disclosure relate to an apparatus that includes a body defining a chamber configured to contain a liquid and having a nozzle located a front end of the body and configured to discharge a stream of the liquid from the chamber toward a surface, a rear wall positioned at a rear of the body and at least partially defining the chamber, an inlet in communication with the chamber and configured for connection to an inlet conduit to introduce an inlet stream of the liquid into the chamber, and an acoustic transducer positioned at the rear wall. The acoustic transducer is configured to generate acoustic energy and to introduce the acoustic energy into the liquid contained in the chamber. The apparatus also includes an exhaust system including a vent in communication with the chamber and configured to remove a mixture of liquid and gas bubbles from the chamber, an exhaust leading away from the vent, and an outlet in communication with the exhaust and configured to discharge the mixture of liquid and gas bubbles into or adjacent to the stream.

According to one aspect, the body is a conical body including a conical wall having a concavely curved portion more proximate to the rear wall and a convexly curved portion between the concavely curved portion and the nozzle, when viewed from inside the chamber, and where an inflection point is located between the concavely curved portion and the convexly curved portion.

According to another aspect, the outlet is positioned to discharge the mixture of liquid and gas bubbles into the stream at a location within the nozzle, or the outlet is positioned to discharge the mixture of liquid and gas bubbles into or adjacent to the stream at a location adjacent to the nozzle.

According to a further aspect, the exhaust system further includes a pump in communication with the exhaust and configured to draw the mixture of liquid and gas bubbles from the vent and to pump the mixture of liquid and gas bubbles to the outlet.

According to yet another aspect, the apparatus includes a manifold located at the rear of the body and connected to an inlet conduit to receive the inlet stream, the manifold further having a plurality of inlets distributed around the manifold and configured for introducing the inlet stream into the chamber. In one embodiment, the vent is formed in the manifold. Additionally, in one embodiment, the manifold includes a central cavity, and the inlets are distributed around the central cavity and configured to introduce the liquid into the central cavity.

According to a still further aspect, the apparatus includes a casing having a handle, where the body, the rear wall, the transducer, the inlet, the vent, and the outlet are positioned within the casing, and the nozzle is positioned to discharge the stream out of the casing. In one embodiment, the exhaust includes an output conduit extending out of the casing and a return conduit extending back into the casing and to the outlet. In this embodiment, the exhaust system may further include a pump in communication with the exhaust and configured to draw the mixture of liquid and gas bubbles from the vent and to pump the mixture of liquid and gas bubbles to the outlet, where the pump is located outside the casing.

Additional aspects of the disclosure relate to an apparatus that includes a conical body defining a chamber configured to contain a liquid and having a nozzle located a front end of the conical body and configured to discharge a stream of the liquid from the chamber toward a surface, a rear wall positioned at a rear of the conical body and at least partially defining the chamber, a manifold located at the rear of the conical body and engaged with the conical body and the rear wall, and an acoustic transducer positioned at the rear wall. The manifold is connected to a liquid supply conduit to receive an inlet stream of the liquid and has a plurality of inlets in communication with the chamber, the inlets being distributed around the manifold and configured for introducing the inlet stream into the chamber, and the acoustic transducer is configured to generate acoustic energy and to introduce the acoustic energy into the liquid contained in the chamber. The apparatus also includes an exhaust system including a vent formed in the manifold in communication with the chamber and configured to remove a mixture of liquid and gas bubbles from the chamber, an output conduit connected to the manifold and leading away from the vent, a pump in communication with the output conduit and configured to draw the mixture of liquid and gas bubbles from the vent via the output conduit, a return conduit in communication with the pump, and an outlet in communication with the return conduit and configured to discharge the mixture of liquid and gas bubbles into or adjacent to the stream at a location adjacent to the nozzle. The pump is further configured to pump the mixture of liquid and gas bubbles to the outlet via the return conduit.

According to one aspect, the manifold includes a central cavity, and the inlets are distributed around the central cavity and configured to introduce the liquid into the central cavity.

According to another aspect, the apparatus includes a casing having a handle, where the conical body, the rear wall, the transducer, the manifold, the vent, and the outlet are positioned within the casing, and the nozzle is positioned to discharge the stream out of the casing. In one embodiment, the output conduit of the exhaust extends out of the casing, and the return conduit extends back into the casing and to the outlet. Additionally, the pump may be located outside the casing.

Other aspects of the disclosure relate to an apparatus that includes a body defining a chamber configured to contain a liquid and having a nozzle located a front end of the body and configured to discharge a stream of the liquid from the chamber toward a surface, a rear wall positioned at a rear of the body and at least partially defining the chamber, a manifold located at the rear of the body and engaged with the body and the rear wall, and an acoustic transducer positioned at the rear wall. The manifold is connected to an inlet conduit to receive an inlet stream of the liquid and has a plurality of inlets in communication with the chamber, the inlets being distributed around the manifold and configured for introducing the inlet stream into the chamber. The manifold further includes a vent in communication with the chamber and configured to remove a mixture of liquid and gas bubbles from the chamber. The acoustic transducer is configured to generate acoustic energy and to introduce the acoustic energy into the liquid contained in the chamber.

Other aspects of the disclosure relate to an apparatus that includes a body defining a chamber configured to contain a liquid and having a nozzle located a front end of the body and configured to discharge a stream of the liquid from the chamber toward a surface, a rear wall positioned at a rear of the body and at least partially defining the chamber, a manifold located at the rear of the body and engaged with the body and the rear wall, and an acoustic transducer positioned at the rear wall. The manifold is connected to an inlet conduit to receive an inlet stream of the liquid and has a plurality of inlets in communication with the chamber, the inlets being distributed around the manifold and configured for introducing the inlet stream into the chamber. The manifold additionally includes a central cavity, and the inlets are distributed around the central cavity and configured to introduce the liquid into the central cavity in a direction parallel to the rear wall. The acoustic transducer is configured to generate acoustic energy and to introduce the acoustic energy into the liquid contained in the chamber.

Other aspects of the disclosure relate to an apparatus that includes a body defining a chamber configured to contain a liquid and having a nozzle located a front end of the body and configured to discharge a stream of the liquid from the chamber toward a surface, a rear wall positioned at a rear of the body and at least partially defining the chamber, a manifold located at the rear of the body and engaged with the body and the rear wall, and an acoustic transducer positioned at the rear wall. The manifold is connected to an inlet conduit to receive an inlet stream of the liquid and has a plurality of inlets in communication with the chamber, the inlets being distributed around the manifold and configured for introducing the inlet stream into the chamber. The manifold also includes a circular central cavity, and the inlets are distributed around the central cavity and configured to introduce the liquid in a radial direction into the central cavity. The acoustic transducer is configured to generate acoustic energy and to introduce the acoustic energy into the liquid contained in the chamber.

Further aspects of the disclosure relate to a system an apparatus as described herein, a control cabinet located remote from the apparatus, and an electrical conduit extending from the control cabinet to the apparatus, where the inlet conduit or the liquid supply conduit extends from the control cabinet to the manifold to supply the inlet stream of the liquid to the manifold, and the electrical conduit is connected to the transducer.

According to one aspect, the control cabinet includes an outgasser in communication with the inlet conduit or the liquid supply conduit and configured to remove gas from the inlet stream. The outgasser may include a micropore filter.

According to another aspect, the pump is located in the control cabinet, and the output conduit and the return conduit extend to and from the control cabinet, respectively.

Still further aspects of the disclosure relate to a system including a liquid supply conduit configured to supply an inlet stream of liquid, a control cabinet including an outgasser in communication with the liquid supply conduit and configured to remove gas from the inlet stream, and a pump, and an apparatus for discharging a stream of the liquid from the chamber toward a surface. The apparatus includes a conical body defining a chamber configured to contain a liquid and having a nozzle located a front end of the conical body and configured to discharge the stream of the liquid from the chamber toward the surface, a rear wall positioned at a rear of the conical body and at least partially defining the chamber, a manifold located at the rear of the conical body and engaged with the conical body and the rear wall, and an acoustic transducer positioned at the rear wall. The manifold is connected to the liquid supply conduit to receive the inlet stream of the liquid and has a plurality of inlets in communication with the chamber, the inlets being distributed around the manifold and configured for introducing the inlet stream into the chamber. The acoustic transducer is configured to generate acoustic energy and to introduce the acoustic energy into the liquid contained in the chamber. The apparatus further includes an exhaust system including a vent formed in the manifold in communication with the chamber and configured to remove a mixture of liquid and gas bubbles from the chamber, an output conduit connected to the manifold and leading away from the vent to the pump, a return conduit in communication with the pump, and an outlet in communication with the return conduit. The pump is configured to draw the mixture of liquid and gas bubbles from the vent via the output conduit and to pump the mixture of liquid and gas bubbles to the outlet via the return conduit, and the outlet is configured to discharge the mixture of liquid and gas bubbles into or adjacent to the stream at a location adjacent to the nozzle.

Other aspects of the disclosure relate to a manifold for use with an apparatus for discharging a stream of liquid from a chamber. The manifold includes a manifold body formed in an annular shape with a central cavity configured to be in communication with the chamber, the manifold body further having a front surface and a rear surface, a vent located at a top of a periphery of the central cavity and configured to remove a mixture of liquid and gas bubbles from the chamber, an exhaust outlet in communication with the vent and configured for connection to an exhaust leading away from the vent, and a manifold inlet configured for connection with a liquid supply to receive an inlet stream of the liquid. The manifold further includes a plurality of conduits extending from the manifold inlet through the manifold body and a plurality of ports distributed around the periphery of the central cavity, where the ports are in communication with the plurality of conduits and are configured introducing the inlet stream into the chamber.

Yet additional aspects of the disclosure relate to an apparatus that includes a conical body defining a chamber configured to contain a liquid and having a base and a nozzle located a front end of the body opposite the base, the conical body configured to discharge a stream of the liquid from the chamber toward a surface, a rear wall positioned proximate the base of the body and at least partially defining the chamber, an inlet in communication with the chamber and configured for connection to an inlet conduit to introduce an inlet stream of the liquid into the chamber, and an acoustic transducer positioned at the rear wall. The acoustic transducer is configured to generate acoustic energy and to introduce the acoustic energy into the liquid contained in the chamber. The conical body includes a conical wall having a concavely curved portion more proximate to the base and a convexly curved portion between the concavely curved portion and the nozzle, when viewed from inside the chamber, and an inflection point is located between the concavely curved portion and the convexly curved portion.

According to one aspect, the conical body further includes a linear portion in which the conical wall narrows in a constant linear manner, the linear portion extending from the convexly curved portion to the front end of the body.

According to another aspect, the conical body is made of a single, molded piece that continuously narrows in diameter from the rear wall to the front end of the body.

According to a further aspect, the conical body has an outwardly extending flange at the base.

Other features and advantages of the disclosure will be apparent from the following description taken in conjunction with the attached drawings.

### BRIEF DESCRIPTION OF THE DRAWINGS

To allow for a more full understanding of the present disclosure, it will now be described by way of example, with reference to the accompanying drawings in which:

FIGS. 1-11 illustrate one embodiment of an apparatus according to aspects of the disclosure;

FIG. 12 illustrates a plot of two embodiments of conical inner wall shapes for use in connection with an apparatus according to aspects of the disclosure;

FIGS. 13-16 illustrate embodiments of different nozzle shapes for use in connection with an apparatus according to aspects of the disclosure;

FIGS. 17-19 illustrate embodiments of conical wall designs for use in connection with an apparatus according to aspects of the disclosure;

FIG. 20 is a schematic view of another embodiment of an apparatus according to aspects of the disclosure;

FIG. 21 is a schematic view of another embodiment of an apparatus according to aspects of the disclosure;

FIG. 22A is a schematic diagram illustrating propagation of acoustic energy through a liquid in an apparatus according to aspects of the disclosure;

FIGS. 22B-C are plots illustrating a model showing a real part of a reflection coefficient and a phase of a reflected wave for multiple different materials, wall thicknesses, and acoustic energy frequencies;

FIGS. 23-26 illustrate one embodiment of a stand for use with an apparatus according to aspects of the disclosure;

FIGS. 27-30 illustrate one embodiment of a conical body including a butterfly valve choke for use with an apparatus according to aspects of the disclosure;

FIG. 31 is a schematic view of another embodiment of an apparatus according to aspects of the disclosure;

FIG. 32 is a schematic view of another embodiment of an apparatus according to aspects of the disclosure;

FIGS. 33-34 illustrate one embodiment of a system including multiple apparatuses according to aspects of the disclosure;

FIGS. 35-38 illustrate another embodiment of a system including multiple apparatuses according to aspects of the disclosure;

FIG. 39 illustrates another embodiment of a system including multiple apparatuses according to aspects of the disclosure;

FIG. 40 illustrates one embodiment of a conveyor cleaning system for use with a system that includes multiple apparatuses according to aspects of the disclosure;

FIGS. 41-44 illustrate one embodiment of a system including a test chamber for use with an apparatus according to aspects of the disclosure;

FIGS. 45-46 illustrate another embodiment of an apparatus according to aspects of the disclosure;

FIGS. 47-52 illustrate another embodiment of an apparatus according to aspects of the disclosure;

FIGS. 53-54 illustrate another embodiment of an apparatus according to aspects of the disclosure, with insets 53A and 54A illustrating portions of the apparatus in greater detail;

FIG. 55 is a diagram illustrating absolute acoustic pressure within a half of a conical body according to aspects of the present disclosure;

FIGS. 56A-C are photographs showing stream characteristics and cleaning performances for removal of waterproof mascara from stainless steel tiles using different pulsing regimes;

FIGS. 56A-C are photographs showing stream characteristics and cleaning performances for removal of waterproof mascara from stainless steel tiles (placed 2 cm downstream from the nozzle) using different pulsing regimes;

FIG. 57A is a plot showing bubble radius vs. frequency to achieve pulsation resonance of air bubbles in water under 1 bar of static pressure;

FIG. 57B is a plot showing the threshold acoustic pressure (dB re 1 μPa) that must be present at a bubble which is assumed to be in pulsation resonance for the frequency in question at the target to stimulate a surface wave of order n, for air bubbles in water under 1 bar of static pressure (for clarity, only a selection of axisymmetric modes are shown);

FIG. 58 shows photographs of samples from cleaning tests using a stream that exits a nozzle having an inner diameter of 2 mm, cleaning mascara foulant that had been allowed to dry on an aluminum bar for 1 minute;

FIG. 59 is a schematic side view of one embodiment of a system that includes multiple apparatuses connected to pipes, according to aspects of the disclosure;

FIG. 60 is a schematic side view of another embodiment of a system that includes multiple apparatuses connected to pipes, according to aspects of the disclosure;

FIG. 61 is a schematic side view of another embodiment of a system that includes an apparatus connected to a pipe, according to aspects of the disclosure;

FIG. 62 is a schematic side view of another embodiment of a system that includes an apparatus connected to a pipe, according to aspects of the disclosure;

FIG. 63 is a schematic side view of one embodiment of an apparatus for cleaning a surface, according to aspects of the disclosure;

FIG. 64 is a schematic side view of another embodiment of an apparatus for cleaning a surface, according to aspects of the disclosure;

FIG. 65A is a perspective view of another embodiment of an apparatus according to aspects of the disclosure;

FIG. 65B is a perspective view of the apparatus of FIG. 65A connected to a container in the form of a water bottle;

FIG. 66 is a schematic side view of another embodiment of an apparatus according to aspects of the disclosure;

FIG. 67 is a schematic side view of another embodiment of an apparatus according to aspects of the disclosure;

FIG. 68 is a schematic side view of another embodiment of an apparatus according to aspects of the disclosure;

FIG. 69 is a schematic side view of another embodiment of an apparatus according to aspects of the disclosure;

FIG. 70 is a schematic side view of another embodiment of an apparatus according to aspects of the disclosure;

FIG. 71 is a schematic side view of one embodiment of a system including an apparatus used with a sink and control cabinet, according to aspects of the disclosure;

FIG. 72 is a schematic side view of another embodiment of a system including an apparatus used with a sink and control cabinet, according to aspects of the disclosure;

FIG. 73A is a schematic side view of one embodiment of a control cabinet that is usable in connection with the systems of FIGS. 71 and 72;

FIG. 73B is a schematic side view of another embodiment of a control cabinet that is usable in connection with the systems of FIGS. 71 and 72, with an inset shown within broken lines;

FIG. 74 is a side view of another embodiment of an apparatus according to aspects of the disclosure;

FIG. 75 is a perspective exploded view of the apparatus of FIG. 74;

FIG. 76 is a cross-sectional view of a conical body of the apparatus of FIG. 74;

FIG. 77 is a rear view of a manifold of the apparatus of FIG. 74;

FIG. 78 is a cross-sectional view of another embodiment of an apparatus according to aspects of the disclosure;

FIG. 79 is a schematic side view of one embodiment of a system including an apparatus provided as a faucet according to aspects of the disclosure;

FIG. 80 is a schematic side view of the apparatus of FIG. 79 and a rotational structure that is usable in connection with the system of FIG. 79;

FIG. 81 is a front view of another embodiment of an apparatus provided as a faucet according to aspects of the disclosure that is usable with the system of FIG. 79;

FIG. 82 is a cross-sectional view of the apparatus of FIG. 81;

FIG. 83 is a front view of another embodiment of an apparatus provided as a faucet according to aspects of the disclosure that is usable with the system of FIG. 79;

FIG. 84 is a cross-sectional view of the apparatus of FIG. 83;

FIG. 85 is a front view of another embodiment of an apparatus provided as a faucet according to aspects of the disclosure that is usable with the system of FIG. 79;

FIG. 86 is a cross-sectional view of the apparatus of FIG. 85;

FIG. 87 is a schematic side view of another embodiment of a system including an apparatus used with a fixture in the form of a water dispenser, according to aspects of the disclosure

FIG. 88 is a schematic side view of an embodiment of a system including an apparatus and a control cabinet, according to aspects of the disclosure;

FIG. 89 is a perspective view of the apparatus of FIG. 88;

FIG. 90 is a perspective view of the apparatus of FIG. 89 with a portion of a casing of the apparatus removed to show internal detail;

FIG. 91 is a cross-section view of the apparatus of FIG. 89;

FIG. 92 is a partial cross-section view of the apparatus of FIG. 89 with arrows indicating flow of fluid through a vent and exhaust of the apparatus;

FIG. 93 is a cross-section view of a portion of the apparatus of FIG. 89;

FIG. 93A illustrates a plot of another embodiment of a conical inner wall shape for use in connection with an apparatus according to aspects of the disclosure;

FIG. 94 is a perspective view of the portion of the apparatus shown in FIG. 93;

FIG. 95 is a rear perspective view of the portion of the apparatus shown in FIG. 93;

FIG. 96 is an exploded rear view of a manifold of the apparatus of FIG. 89;

FIG. 97 is a rear perspective view of the manifold of the apparatus of FIG. 89;

FIG. 98 is a bottom rear perspective view of the manifold of the apparatus of FIG. 89;

FIG. 99 is a cross-section view taken along lines 99-99 of FIG. 98;

FIG. 100 is a cross-section view taken along lines 100-100 of FIG. 98;

FIG. 101 is a top view of the apparatus of FIG. 89 with laser beams shown schematically;

FIG. 102 is a perspective view of the control cabinet of FIG. 88;

FIG. 103 is a cross-section view of the control cabinet of FIG. 88;

FIG. 104 is a perspective view of the control cabinet of FIG. 88, with a portion of a frame of the control cabinet removed to show internal detail, and with arrows schematically illustrating flow of liquid into and out of the control cabinet;

FIG. 105 is a partially exploded perspective view of the control cabinet of FIG. 88;

FIG. 106 is a rear perspective view of the control cabinet of FIG. 88, with a portion of the control cabinet removed to show internal detail;

FIG. 107 is a partially exploded perspective view of the control cabinet of FIG. 88; and

FIG. 108 is a plan view of a portion of the control cabinet of FIG. 88, with arrows schematically illustrating flow of liquid through the components depicted.

### DETAILED DESCRIPTION

While this invention is capable of embodiments in many different forms, there are shown in the drawings and will herein be described in detail example embodiments of the invention with the understanding that the present disclosure is to be considered as an exemplification of the principles of the invention and is not intended to limit the broad aspect of the invention to the embodiments illustrated. In the following description of various example structures according to the invention, reference is made to the accompanying drawings, which form a part hereof, and in which are shown by way of illustration various example devices, systems, and environments in which aspects of the invention may be practiced. It is to be understood that other specific arrangements of parts, example devices, systems, and environments may be utilized and structural and functional modifications may be made without departing from the scope of the present invention.

In this specification, the term “wound” is defined as including (but is not restricted to) sites formed by the removal or transformation or inflammation of the normal human or animal tissue (epidermis, gum etc.) to produce abnormal exposure of underlying tissue, or transform healthy tissue into unhealthy tissue. Trauma, burning, sun exposure, cutting, the formation of ulcers and abscesses, disease (including gum disease) are all included. Specific circumstances would be abrasion or cutting or burning or solar exposure of the epidermis to expose the dermis; or damage to the gum. Additionally, in this specification, the term “anatomical pocket” is defined as including (but is not restricted to) periodontal pockets, cavities associated with the eye, the urinary-genital system, ears, and oral, nasal and digestive systems, other anatomical cavities or spaces, or wound crevices.

In some embodiments, the stream of liquid for therapeutic use can be chemical free, and may comprise or consist of water, optionally in the form of a conventional saline solution which in this context means that the solution is approximately isotonic with human tissue fluid, or solution of sodium chloride 0.9% w/v, and may be free of biocides and/or drugs or other pharmaceutical compositions. The use of water or a saline solution for cleaning reduces the risk of adverse reactions in tissue, such as tissue that has been severely traumatized (e.g. burns) or as a result of allergic reaction or damage through repeated chemical use (e.g., hand sanitizer). The use of water or a saline solution for cleaning also reduces the provision in waste of dilute forms of pharmaceuticals (such as antibiotics) which are known to contribute to the development of antimicrobial resistance (such as antibiotic resistance in bacteria, the resistance of viruses to antivirals, the resistance of fungi to antifungals, and the resistance of parasites to the drugs used against them).

Generally, this disclosure relates to an apparatus and method for delivering one or more streams of water or other liquid (which may, in some embodiments, carry active agents such as biocides, cleaning agents, drugs, etc.) and acoustically activated bubbles to a target to provide beneficial effects (e.g., rinsing and cleaning) to the target. The stream(s) individually or collectively include acoustic waves (e.g., ultrasound) and bubbles of a suitable size and concentration to be effective in cleaning the target and/or producing other beneficial effects. The general principle supporting the efficacy of this apparatus and method is the delivery of bubbles of a suitable size at a suitable time to produce beneficial effects at the target and acoustic waves that engage the bubbles at or near the target to produce these beneficial effects. If a gas bubble in a liquid is subjected to the oscillating pressure field of an acoustic wave, it pulsates with close to spherical symmetry. However, if the conditions are tuned properly, the acoustic wave can cause tiny asymmetries on the bubble wall to grow very rapidly during this pulsation, so that instead of dissipating, these bubble wall instabilities grow to form surface waves that ripple across the bubble wall, having amplitudes that can be 1-4 orders of magnitude (usually 2-3 orders of magnitude) higher than the amplitude of the original spherical pulsation, and usually have frequencies of around half that of the spherical pulsation.

As used herein, the term “resonant bubbles” refers to bubbles that are of a size that is sufficiently close to the bubble size that would be in pulsation resonance with the sound field to have these surface waves excited upon their individual bubble walls, provided that the amplitude of the acoustic field at the location (typically at the target) of the bubble of relevance exceeds the threshold required to excite these surface waves, for bubbles of this size and an acoustic wave of this frequency. Bubbles that are either too large or too small to have such surface waves stimulated on them, in the sound field (given its frequency and amplitude) that is present at the target, are referred to herein as “unwanted bubbles” or “sub-optimal bubbles.” According to one embodiment, the apparatus and method described herein are configured such that resonant bubbles and acoustic waves arrive at the target at the same optimal time. According to an additional embodiment, the apparatus and method described herein are configured such that the number of unwanted bubbles is minimized and such that the number of bubbles in the travel path of the acoustic waves to the target is minimized.

These surface waves on the bubble wall generate a number of actions that produce beneficial effects associated with the target. For example, these surface waves produce liquid shear and flow (often circulating flow) in the liquid close to the bubble, i.e. from next to the bubble wall to a distance of up to 50 bubble radii, and this shear and flow produce beneficial effects associated with the target. One such effect is the removal of contaminants adhered to the target, and it has been found that such acoustically-excited bubbles clean surfaces more gently (i.e., without causing damage to the target) than can the cavitation generated in ultrasonic baths. Other such beneficial effects include wound healing and other forms of regeneration and/or treatment. As one example, the shear and secondary acoustic waves generated in an anatomical pocket can achieve the removal of microbes without compromising the host immune system (e.g. of the plant or patient, be it human or animal). As another example, the convection of material in and out of the anatomical pocket, resulting from such shear and flow, would flush fresh water/saline solution and any chemical agent (e.g., biocide) or drug from the solution into the pocket which would not normally penetrate so rapidly, or in such high concentrations, by diffusion alone. Effects such as ultrasonically induced microstreaming, convection, and bubble wake effects promote penetration of any such additives into pockets or crevices where they would normally not penetrate. Additionally, after microbes have been dislodged, e.g., by bubble-generated shear, the convection would flush them out of the wound crevice. As a further example, many cells in a wound, including keratinocytes and fibroblasts, are mechano-transducers. Such cells will respond to the mechanical forces generated by the shear and flow by up-regulating production of the chemical signals such as growth factors that accelerate the healing process. Similarly, the forces exerted by the presence of the bubbles stretch activated cell wall receptors, stimulating the release of intracellular signals that in turn up-regulate the expression of genes and improve healing.

Other examples of beneficial actions generated by the surface waves on the bubble wall include hydrodynamic pressure fluctuations close to the bubbles and acoustic signals generated and scattered by the bubble. These are generated even if the entire system is simply two-phase (bubble gas and liquid). Another such beneficial action includes mixing, for example, to avoid deleterious effects that can occur in the absence of mixing. An example of such a deleterious effect is if the target is interacting through the generation or transfer of chemical species into and out of the liquid, this action may form a depletion layer adjacent to the target in the liquid (e.g., a layer that is depleted of dissolved or chemical species that have been used up by the target, such as dissolved oxygen, chemical species, reactive agents), or a liquid layer that contains an elevated concentration of species generated by or emitted from the target (e.g. CO₂ from the respiring living tissue layer). These strong concentration gradients caused by species that are depleted or elevated in the liquid layer next to the target can be reduced, often in a beneficial way, to remove the unwanted excess or depletion of chemical species, by mixing the liquid close to the surface (where the strong concentration gradient occurs) with the liquid further from the target. Such mixing can increase the efficiency of reaction at, or transfer across the interface with, the target, by the circulation currents set up by the surface waves on the bubble wall and by the motion of bubbles towards the target under the influence of radiation forces. In this way, the liquid close to the surface is refreshed by mixing in liquid from further away from the surface.

Still further beneficial actions can be created by addition of chemicals to the liquid as discussed elsewhere herein. Such chemicals may be doped or built on or within the bubble wall, including in the form of distinct chemically-distinctive layers forming that wall, both to stabilize the wall and to impact a chemical or biological effect upon the target. Such chemicals could additionally or alternately be placed within the bubble gas. These chemicals may impart change upon the target and/or affect (e.g., stabilize) the fragmentation or dissolution of the bubble.

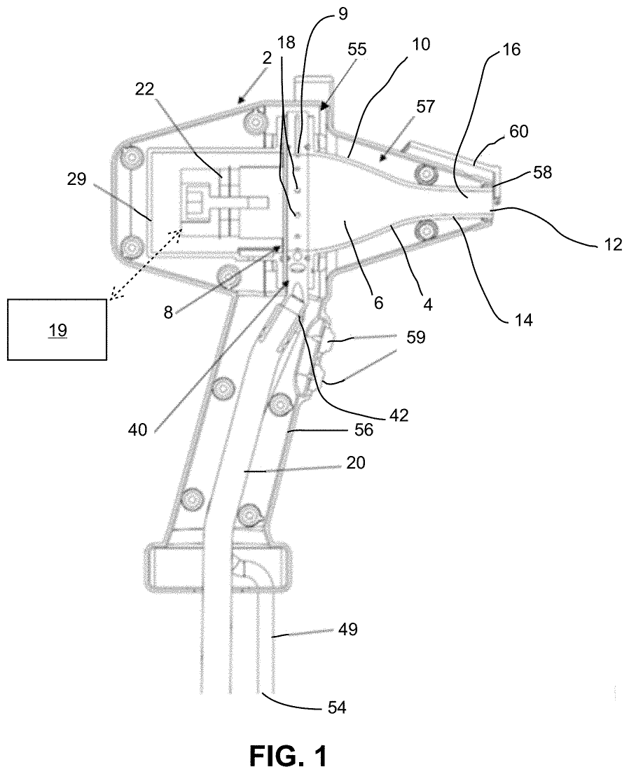

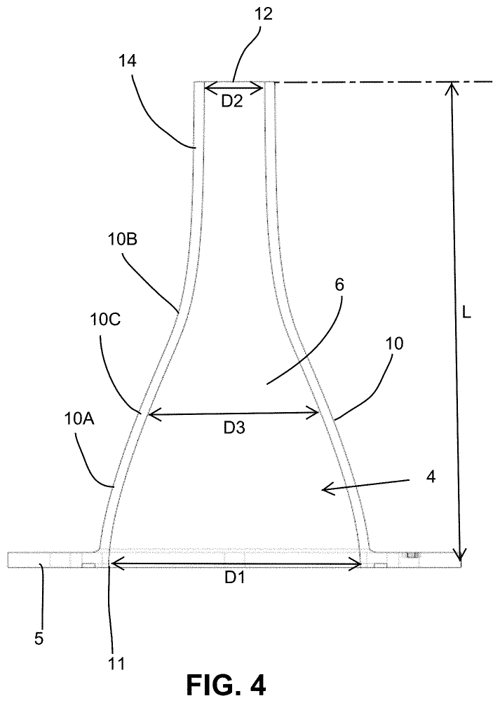

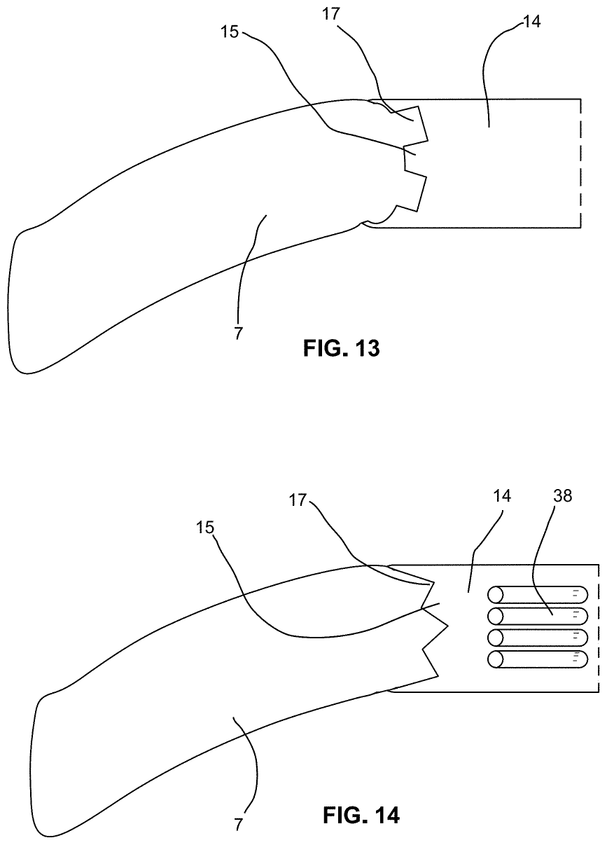

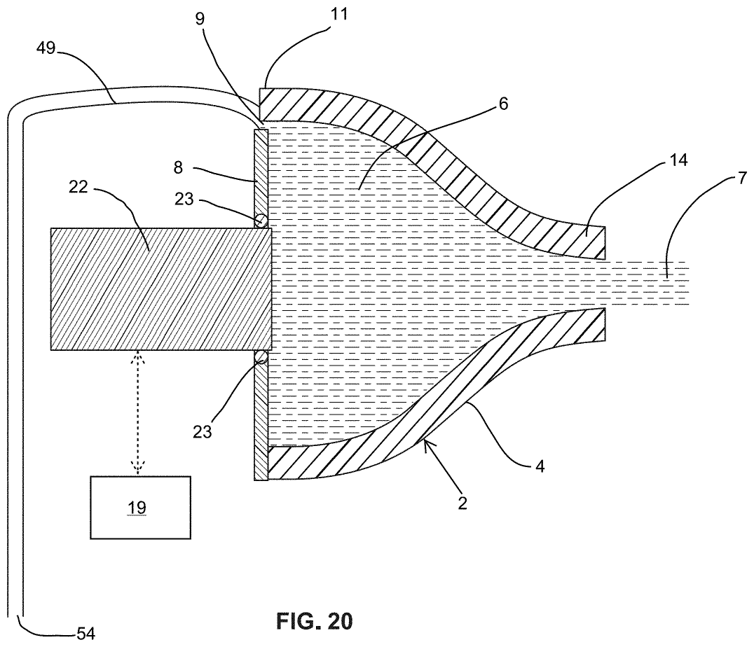

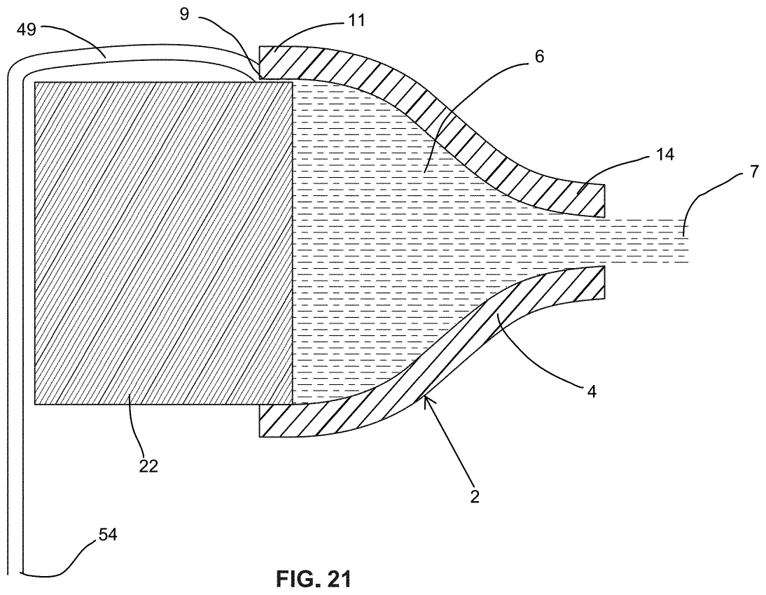

FIGS. 1-11 illustrate one general embodiment of an apparatus 2 for delivering a stream of liquid including bubbles and acoustic waves to a target. The apparatus 2 can be used in any orientation, and some orientations may benefit from a venting or exhaust system and/or a choking mechanism, as described herein. The apparatus 2 in this embodiment includes a hollow conical body 4 defining a chamber 6. During operation, this will be filled with liquid that issues from the nozzle 14 as a stream (as shown by the dotted region in FIGS. 20-21). The body 4 has a base 11, with a rear wall 8 located at the base 11 and a substantially conical wall 10 extending forwardly away from the rear wall 8. The conical wall 10 terminates in an outlet nozzle 14 at an outlet 16 of the conical body 4, and the nozzle 14 includes an orifice 12 through which the liquid exits the chamber 6. The apparatus 2 also includes one or more vents 9 through which liquid containing any gas pockets can leave. In the embodiment of FIGS. 1-11, the apparatus 2 has a vent 9 located near the manifold 40 or as a part of the manifold 40 and/or near the rear wall 8. This location that is configured to be the uppermost point of the liquid in the chamber 6 during use in which the apparatus 2 is oriented so the nozzle 14 points downwardly, horizontally, or at some angle in between. In the embodiment of FIGS. 1-11, the uppermost point of the liquid in the chamber 6 during use in this orientation is generally where the portion of the rear wall 8 opposite the manifold inlet 42 meets the portion of the substantially conical wall 10 opposite the orifice 12. Both the substantially conical wall 10 and the outlet nozzle 14 in FIGS. 1-11 are rotationally symmetric, i.e. circular, although other geometric shapes may be employed. For example, FIGS. 13-16 depict some additional embodiments of configurations for the outlet nozzle 14 described elsewhere herein, some of which may not be rotationally symmetric. As used in this specification, the term “conical” should be interpreted broadly to encompass structures that have a narrowing width/diameter and cross-sectional area along the length thereof. The term “conical” therefore encompasses structures which are not only geometrically conical, and for example have a linear, convex or concave wall, but also structures which for example are bell-like and have a concave contour as seen from inside the chamber 6, or have a constant half-angle as shown, or are horn-like and have a convex contour as seen from the inside the chamber 6, or have a combination of surface contours, such as having a combination of concave and convex contours when seen from inside the chamber 6.

The apparatus 2 further includes an acoustic transducer 22 positioned at or adjacent to the rear wall 8 and configured to introduce acoustic energy into the liquid within the chamber 6. The acoustic transducer 22 is mounted on the rear wall 8 in the embodiment of FIGS. 1-11. A controller 19 (shown schematically in FIGS. 1 and 20) controls the operation of the transducer 22. The controller 19 may perform signal generation, including its form, temporal profile and frequency content, the timing of any pulsing of the signal and its relationship to the timing of the bubble generation signal, and the amplitude of the signal as supplied to the power amplifier, which may also be included in the controller 19. Typically, the transducer 22 is mounted on an outer surface of the wall 8 and extends over a substantial proportion of the surface area of the wall 8. Alternatively, the transducer 22 may be embedded into the chamber 6 on or through the rear wall 8. FIG. 20 illustrates an embodiment where the transducer 22 is embedded through the rear wall 8 with seals 23 between the transducer 22 and the rear wall 8, and FIG. 21 illustrates an embodiment where the transducer 22 replaces the rear wall 8 entirely and occupies the space that is occupied by the rear wall 8 in other embodiments. The transducer 22 may be mounted elsewhere at a location associated with conical body 4 provided that the transducer 22 is configured to introduce acoustic energy into the liquid within the chamber 6 whereby the acoustic energy is present in an output stream from the nozzle 14.

In some embodiments, an outgasser may be used for the liquid supplied to the conical body 4 to reduce the build-up of gas within the conical body 4, such as an outgasser that uses a microporous filter. Other example embodiments of outgassers that may be used in connection with the apparatus 2 as described herein are shown and described in International Publication No. WO 2018/228848, which is incorporated by reference herein. Nevertheless, certain circumstances (for example, prolonged use, air leaks in the pump, insufficiently smooth pumping, variation in the gas content of the water coming from the source, etc.) can cause gas to build up in the chamber 6 that needs to be vented. Thus, the apparatus 2 may include both an outgasser and one or more vents 9 in one embodiment.

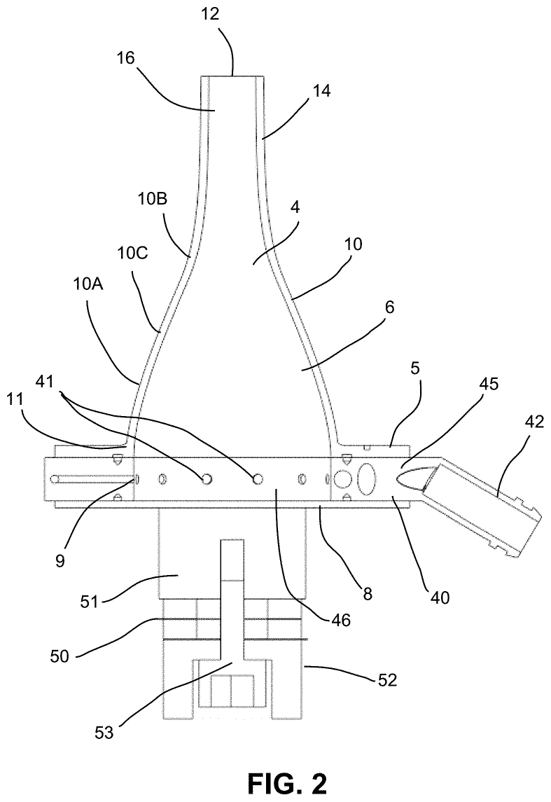

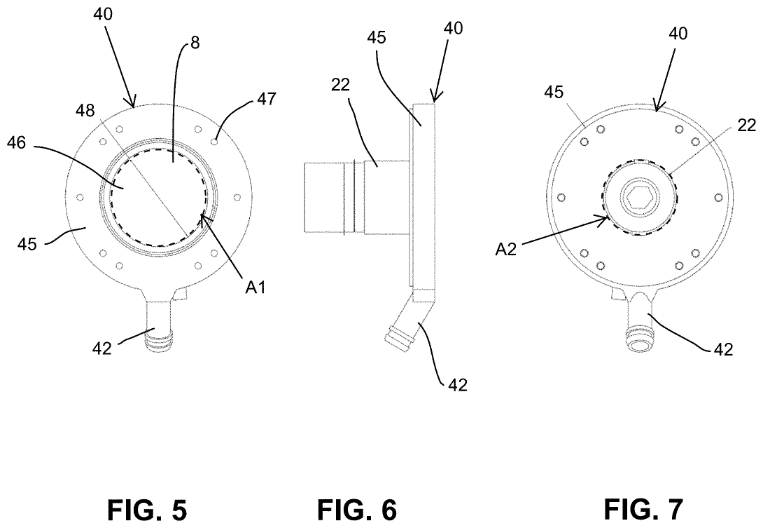

The conical body 4 also includes one or more liquid inlets 18 located at or adjacent to the rear wall 8. In the embodiment of FIGS. 1-11, the apparatus 2 includes a manifold 40 located at the base 11 of the body 4 that includes a manifold body 45 having multiple ports 41 that operate as the inlets 18 of the body 4. In general, the manifold 40 includes a manifold inlet 42 that is connected to a liquid supply conduit 20 in communication with a source/supply of liquid (not shown) and one or more internal conduits 44 that extend from the manifold inlet 42 through the manifold body 45 to the plurality of ports 41 to distribute the liquid flow from the manifold inlet 42 to the ports 41, where the liquid enters the body 4. The liquid supply conduit 20 is typically in the form of a flexible hose and forms part of a liquid supply system that also includes the liquid source (not shown). Rigid piping may be used for the supply conduit 20 in some embodiments, such as if the apparatus is mounted as part of a fixture, e.g., as a faucet. FIGS. 5-8 illustrate the manifold 40 of the embodiment of FIGS. 1-11 in greater detail. As shown in FIGS. 1-2, the manifold 40 is connected directly to the base 11 of the body 4 by connection of the manifold body 45 to a flange 5 that extends outward around the base 11. The manifold 40 is also connected directly to the rear wall 8 by connection of the manifold body 45 to the rear wall 8, such that a portion of the manifold body 45 is sandwiched between the rear wall 8 and the flange 5 of the base 11. The manifold body 45 includes a plurality of holes 47 that receive fasteners for connection to the base 11 and the rear wall 8. The manifold inlet 42 is in the form of a tube that extends downwardly from the bottom side of the manifold body 45.

In one embodiment, the apparatus 2 is configured so that the inlet(s) 18 introduce the liquid into the chamber 6 in a flow direction that is radially inward with respect to the periphery of the conical wall 10 and parallel to the rear wall 8 and/or parallel to the face 25 of the transducer 22. In the embodiment of FIGS. 1-11, the manifold body 45 is formed in an annular shape with a central cavity 46, and the ports 41 of the manifold 40 are directed to introduce the liquid radially inwardly into the central cavity 46. In this configuration, the periphery 48 of the central cavity 46 partially defines the chamber 6, and the central cavity 46 exposes a portion of the rear wall 8. The direction of the liquid flow in this embodiment assists in removing bubbles that may adhere to the rear wall 8. The internal conduits 44 in the embodiment of FIGS. 1-11 include two branch conduits 44A that branch in diverging directions away from the manifold inlet 42, and a plurality of intermediate conduits 44B extending from the branch conduits 44A around the periphery of the central cavity 46 to a plurality of distribution chambers 44C. The distribution chambers 44C are positioned immediately surrounding the central cavity 46, with each distribution chamber 44C having a plurality of ports 41. The ports 41 may be distributed at regular intervals around the central cavity 46 in one configuration. In the embodiment of FIGS. 1-11, the manifold 40 has two symmetrical intermediate conduits 44B and four symmetrical distribution chambers 44C, with each distribution chamber 44C extending around approximately ¼ of the periphery 48 of the central cavity 46 and each distribution chamber 44C having three ports 41. In this configuration, the twelve total ports 41 are distributed at approximately 30° intervals around the central cavity 46. The use of multiple, evenly distributed inlets 18 reduces the amount of hydrodynamic turbulence within the chamber 6 and ensures a symmetric flow pattern to match the symmetric acoustic field generated by the transducer 22. In particular, the turbulence is reduced compared to a configuration having fewer, larger inlets 18 because smaller length-scales of turbulence have less energy and are more rapidly dissipated by the liquid viscosity.

The manifold 40 also includes a vent 9 positioned at or near the end of the manifold body 45 opposite the manifold inlet 42. In this location, the vent 9 is configured to be positioned at the uppermost level of the liquid in the chamber 6 during use in which the apparatus 2 is oriented so the nozzle 14 points downwardly, horizontally, or at some angle in between. The vent 9 opens to the central cavity 46 and is thereby in communication with the chamber 6. The manifold 40 also includes a gas conduit 43 extending from the vent 9 through the manifold body 45 to an exhaust 49 connected to the manifold 40 near the manifold inlet 42 at the bottom of the manifold body 45.

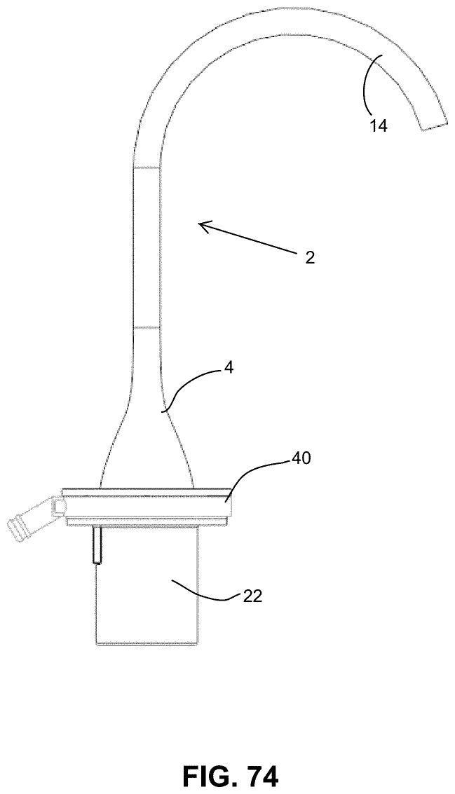

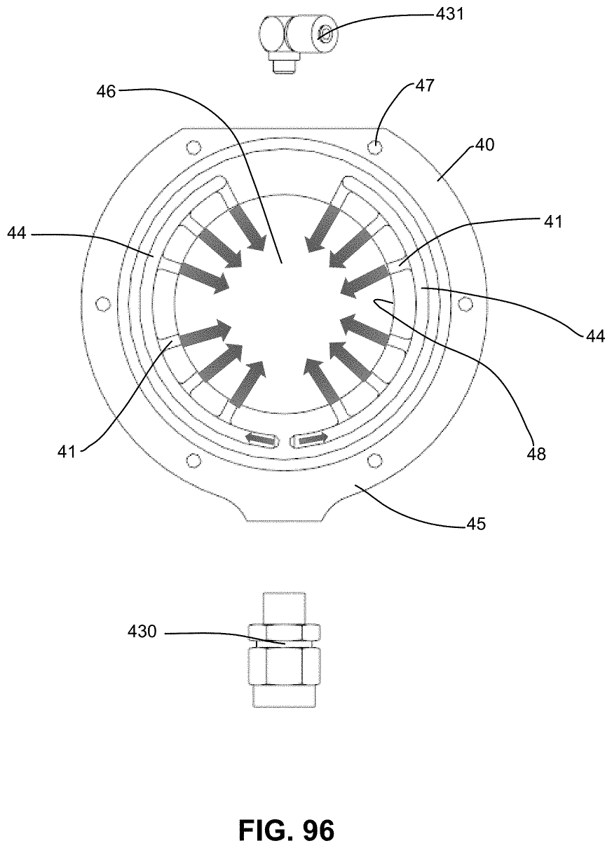

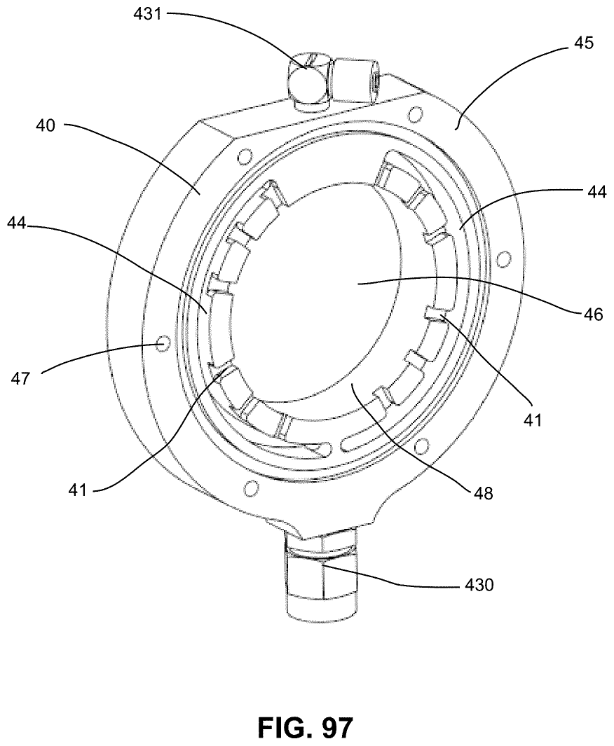

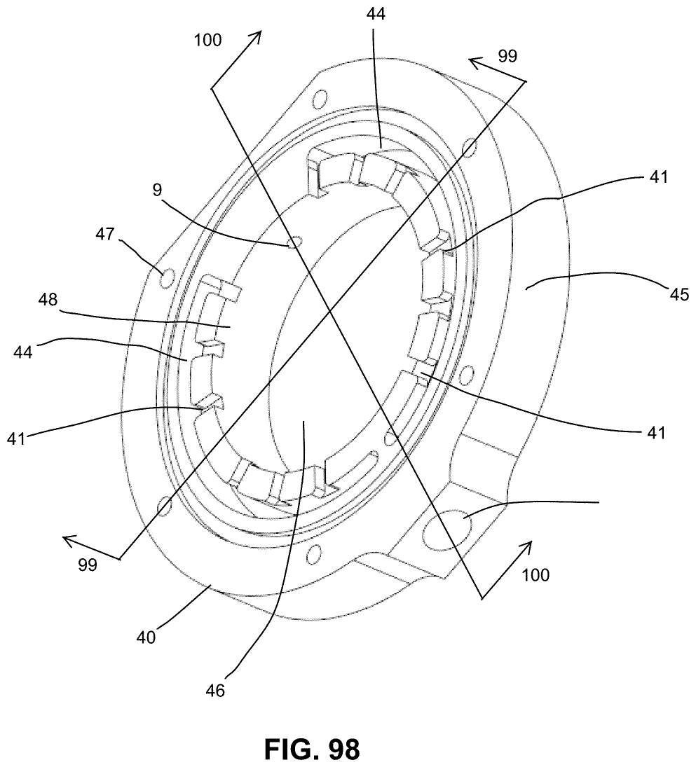

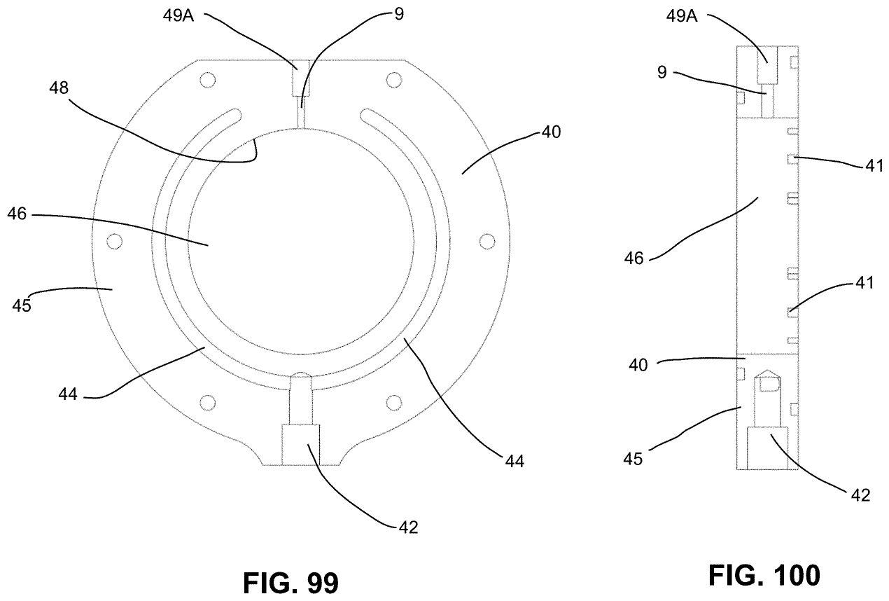

It should be noted that the embodiment of the manifold 40 shown in FIGS. 1-11 is only one potential embodiment. As discussed herein, FIGS. 74-78 and FIGS. 90-100 illustrate additional embodiments of manifolds 40. Additional features may be included in further embodiments, which may potentially improve functionality for the manifold 40. For example, in one embodiment, the corners in and between the conduits 44 within the manifold body 45 may be smoother and/or more rounded, in order to promote a smoother flow path through the conduits 44, which may include replacing sharper corners with gently rounded bends of large bend radius. In another embodiment, the ports 41 may have different sizes and/or spacing depending on their distances from the manifold inlet 42, in order to reduce the tendency for greater flow through the ports 41 closer to the manifold inlet 42. As one example, the ports 41 may be configured to have larger sizes (i.e., widths) at greater distances from the manifold inlet 42 in one embodiment. As another example, the ports 41 furthest from the manifold inlet 42 may have a total cross-sectional area that is smaller than that of the conduits 44 extending to such ports 41, as this creates a small pressure drop across the ports 41 and a higher flow resistance across the ports 41. In another embodiment, the ports 41 may be directed such that the liquid exits the ports 41 in an angled (non-radial) direction to generate a swirl or vortex in the chamber 6 to assist with the removal of bubbles from the chamber 6. Further features and modifications may be provided in other embodiments.

Each embodiment of the manifold 40 as described herein (including the embodiments in FIGS. 1-11, FIGS. 74-78, and FIGS. 90-100, may be used in connection with any other embodiment described herein. It is understood that in some embodiments herein, such as those in FIGS. 20-21 and 31-32, the drawings are depicted schematically, and the manifold 40 (or other liquid inlet 18) is not illustrated for the sake of simplicity. Nevertheless, such embodiments are contemplated for use with a manifold 40 or other liquid inlet(s) 18.

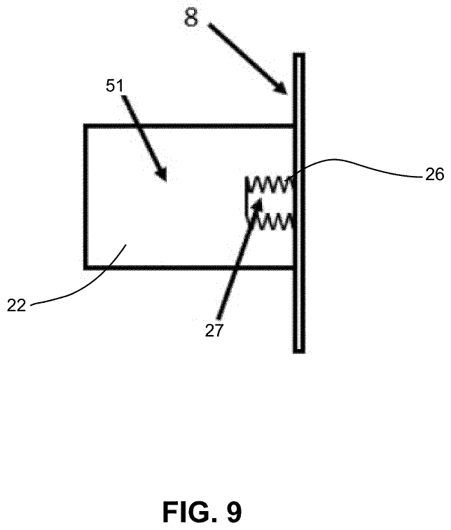

The rear wall 8 is positioned at the base 11 of the body 4 and defines at least a portion of the rear of the chamber 6. The rear wall 8 comprises a plate, for example of plastics/polymers, such as polycarbonate, acrylic, or rubber, or of a metal such as aluminum, brass, stainless steel, or copper. The material may be provided with anti-microbial properties as well (e.g., copper), which may be useful in clinical or other settings. It is understood that the rear wall 8 may be made from multiple materials. In one embodiment, the acoustic transducer 22 is mounted on the rear wall 8 or otherwise connected to the rear wall 8. In the embodiment of FIGS. 1-11, the transducer 22 is connected to the rear side of the rear wall 8, e.g. by a bonding material or technique such as an adhesive that will not delaminate or fracture under the expected usage, welding, brazing, soldering, etc. and/or by a mechanical connection. FIG. 9 illustrates one embodiment of a mechanical connection where the transducer 22 (e.g., the head mass 51) has a female threaded hole 27 that receives a bolt or threaded stud 26 that is attached or welded to the rear wall 8. It is understood that these structures may be transposed in another embodiment. In other embodiments, as described elsewhere herein, the transducer 22 may extend through a hole in the rear wall 8 (or multiple holes if multiple transducers 22 are used), such as in FIG. 20, or the transducer 22 may be connected directly to the base 11 of the conical body 4 without a rear wall 8 being present, such as in FIG. 21. In a further embodiment, the transducer 22 may be directly connected to the manifold body 45 and positioned within or adjacent to the central cavity 46. The rear wall 8 in FIGS. 1-11 is connected to the rear side of the manifold body 45 as described herein and covers the central cavity 46 of the manifold 40 to define the rear end of the chamber 6. In this configuration, the liquid inlets 18 are directed parallel and adjacent to the portions of the rear wall 8 exposed within the central cavity 46 of the manifold 40. An inner housing 29 is also connected to the rear side of the rear wall 8 to enclose the transducer 22. In another embodiment, multiple transducers 22 may be connected to the rear wall 8 or otherwise configured to pass acoustic energy through the rear wall 8, as described below.

The body 4 extends forwardly of the rear wall 8 to the nozzle 14, and the nozzle 14 may be integral with the end of the conical body 4, as in the embodiment of FIGS. 1-11. The body 4 generally has a conical structure as defined herein, which structure may also be rotationally-symmetrical when viewed along the length thereof. In other embodiments, non-symmetrical shapes may be used, such as shapes that reflect and merge into a letter-box-shaped or elliptical nozzle. Generally, the base 11 of the body (having the largest width/diameter) will be at least as large as the diameter/width of the face of the transducer 22, and the nozzle 14 will have a smaller diameter than the base 11. The diameter of the nozzle 14 may be configured based on the minimum frequency at which sound will pass down the stream Table 1 below reports such minimum acoustic wave frequencies for various liquid media (having different sound speeds) and nozzle diameters.

TABLE 1

| | Liquid | | | |
|---|---|---|---|---|
| | Bubble-free | Water with void | Water with void | Bubble-free |
| | water | fraction of 0.003%* | fraction of 0.006%* | liquid mercury |
| | Sound speed (m/s) | | | |
| | 1481 | 1224 | 1067 | 1450 |
| Nozzle diameter (mm) | Cut-off frequency (kHz) (the minimum operating ultrasonic carrier frequency to transmit sound into the stream) | | | |
| 30 | 38 | 31 | 27 | 37 |
| 20 | 57 | 47 | 41 | 55 |
| 10 | 113 | 94 | 82 | 111 |
| 4 | 283 | 234 | 204 | 277 |
| 2 | 567 | 468 | 408 | 555 |
| 1 | 1134 | 937 | 817 | 1110 |

*at frequencies far below bubble resonance, 20° C. and 1 atm. pressure.

Table 2 below reports the minimum nozzle diameter required to permit acoustic energy to propagate through the stream for various liquid media and acoustic wave frequencies.

TABLE 2

| | Liquid | | | |
|---|---|---|---|---|
| | Bubble-free | Water with void | Water with void | Bubble-free |
| | water | fraction of 0.003%* | fraction of 0.006%* | liquid mercury |
| | Sound speed (m/s) | | | |
| | 1481 | 1224 | 1067 | 1450 |
| Frequency (kHz) | Cut-off diameter (mm) (the minimum nozzle diameter to transmit sound into the stream) | | | |
| 70 | 16.2 | 13.4 | 11.7 | 15.9 |
| 135 | 8.4 | 6.9 | 6.1 | 8.2 |
| 160 | 7.1 | 5.9 | 5.1 | 6.9 |
| 200 | 5.7 | 4.7 | 4.1 | 5.5 |
| 500 | 2.3 | 1.9 | 1.6 | 2.2 |
| 1000 | 1.1 | 0.9 | 0.8 | 1.1 |
| 3000 | 0.4 | 0.3 | 0.3 | 0.4 |

*at frequencies far below bubble resonance, 20° C. and 1 atm. pressure.

It is noted that the minimum frequency that will propagate through the stream is determined by the stream diameter and not the diameter of the nozzle 14 itself as expressed in Tables 1 and 2. Nevertheless, the stream diameter at the nozzle 14 is determined by the diameter of the nozzle 14, and thus, the nozzle 14 may be designed based on the calculations and figures set forth in Tables 1 and 2. Therefore, Tables 1 and 2 show how selection of the acoustic wave frequency places a lower limit on the nozzle 14 diameter that will allow the acoustic energy to enter into the stream and propagate away from the nozzle 14, depending on the sound speed of the liquid. It is noted that the calculations in Tables 1 and 2 assume a nozzle of circular cross-section; nozzles and streams of different cross-sections (e.g. letter-box shaped) will follow similar rules but with specific values related to the stream. In a liquid stream with pressure-release walls, the narrowest dimension plays a dominant role in determining the cut-off frequency.

In Tables 1 and 2, the mapping between nozzle 14 diameters and minimum operating frequencies (Table 1), and between the operating frequency and the minimum nozzle 14 diameter (Table 2), depends on the sound speed in the liquid. The sound speed in the liquid depends not only on liquid identity, but can also be strongly influenced by the size distribution and void fraction (the percentage of bubbly water that is free gas) within it. Four examples (one of bubble-free water, two of bubbly water of differing void fractions assuming all bubbles are much smaller than the size that would be in pulsation resonance with the ultrasound, and one of bubble-free mercury) that are not exclusive, but are illustrative, are shown in Tables 1 and 2 for 20° C. and 1 atmosphere of static pressure.

As expressed in Table 1, once the nozzle diameter has been chosen, this structure (e.g., diameter) of the nozzle 14 places a lower limit on the acoustic wave frequency that will enter the stream and propagate away from the nozzle 14, depending on the sound speed of the liquid. For the data presented in Table 2, it is noted that higher frequencies than the minimum values in Table 1 can propagate into the stream for a given nozzle 14 diameter and sound speed, until such point when the stream thins to a width that makes that acoustic frequency evanescent at that particular location in the stream.

As expressed in Table 2, once the acoustic wave frequency has been chosen, for a liquid with a given sound speed, this determines the minimum stream thickness and nozzle diameter 14 through which this acoustic energy will propagate. For the data presented in Table 2, it is noted that wider nozzles 14 allow the acoustic energy to propagate into the stream for a given frequency and sound speed, until such point when the stream thins to a width that makes that acoustic wave frequency evanescent at that particular location in the stream.

In one embodiment, the body 4 has a circular cross-sectional shape that tapers in width from the base 11 to the nozzle 14. In a further embodiment, the conical wall 10 of the body 4 has a rotationally symmetrical shape about the length or axial direction and has a concave portion 10A closer to the rear wall 8 and a convex portion 10B between the concave portion 10A and the nozzle 14, when viewed from inside the chamber 6, and may also include an inflection point 10C located between the concave portion 10A and the convex portion 10B. The curved shape of the body 4 in this embodiment prevents a sudden change in cross section.

The body 4 in FIGS. 1-4 has a length L of 96.5 mm, a diameter D1 at the base 11 of 50 mm, a diameter D2 at the nozzle 14 of 12 mm, and a wall thickness of 2 mm, and the inflection point 10C is located 44.8 mm from the base 11 of the body 4, with a diameter D3 at the inflection point 10C of 22.2 mm. The body 4 also has an internal chamber 6 volume V of 64,705 mm³ or 64.705 ml, and the rear wall 8 has a diameter of 90 mm, with an area A1 having a diameter of 50 mm and a surface area of 1,963 mm² being exposed to the chamber 6. In other embodiments, the body 4 may have a structural configuration with dimensions that are within +/−10% of these values or +/−5% of these values. The apparatus 2 shown in FIGS. 1-4 is configured for producing a 135 kHz acoustic frequency, and these dimensions are based on that. The minimum nozzle 14 diameter for this configuration would be 8.4 mm, as shown in Table 2. In other embodiments, the body 4 may be scaled in proportion to the properties (e.g., size) of the transducer 22, the diameter of which does not scale with frequency, and the cut-off diameter (as noted in Table 2). The minimum nozzle 14 diameter therefore does scale approximately with the inverse of the acoustic wave frequency, but the transducer 22 diameter may change far less. Once scaled for those factors, the dimensions and frequencies used may be within +/−10% of these values or +/−5% of these values in various embodiments.

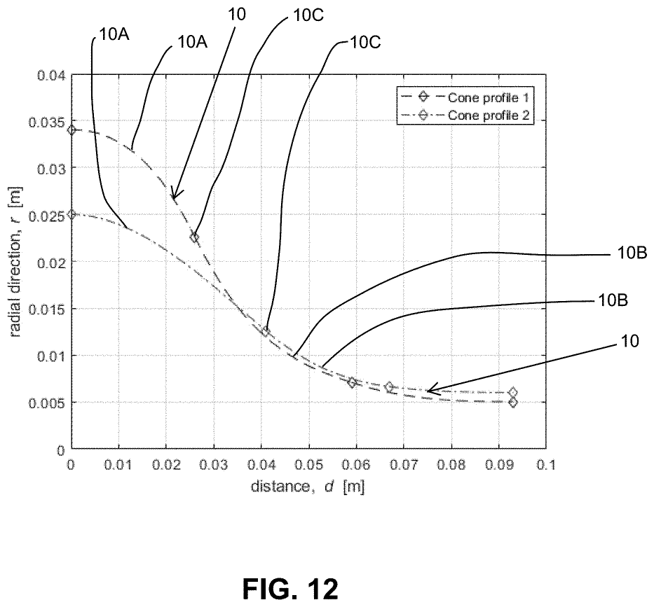

FIG. 12 illustrates two potential sidewall profiles for the body 4 that include such a concave portion 10A and a convex portion 10B, which present particular advantages by minimizing the amount of standing water in the chamber 6 and transmitting maximum acoustic energy into the liquid stream 7. In FIG. 12, the “r” and “d” values represent the vertical and horizontal coordinates, respectively, in meters of the inner surface of the wall 10, assuming the axis of rotational symmetry is horizontal and lies on the axis defined by r=0. The diamonds signify the points where the radius and slope are defined. These profiles are defined by piece-wise third-order polynomial expressions based on the radius and slope at four distances shown in Table 3. The radius values between these distances can be determined by the unique third-order polynomial with the slopes and radius' in the Table 3. At each distance d between two points d(1) and d(2) (values from adjacent rows in Table 3) the radius can be determined by a polynomial of the following form:

r=A₁(d−d_{mid})³+A₂(d−d_{mid})²+A₁(d−d_{mid})+A₄,

where d_{mid} is the average of d(1) and d(2) and the A coefficients can be determined by solving the following matrix operation:

$$( \begin{matrix}r ( 1 ) \\ r ( 2 ) \\ S ( 1 ) \\ S ( 2 )\end{matrix} ) = ( \begin{matrix}{( d ( 1 ) - {d}_{mid} )}^{3} & {( d ( 1 ) - {d}_{mid} )}^{2} & ( d ( 1 ) - {d}_{mid} ) & 1 \\ {( d ( 2 ) - {d}_{mid} )}^{3} & {( d ( 1 ) - {d}_{mid} )}^{2} & ( d ( 1 ) - {d}_{mid} ) & 1 \\ 3 \cdot {( d ( 1 ) - {d}_{mid} )}^{2} & 2 \cdot ( d ( 1 ) - {d}_{mid} ) & 1 & 0 \\ 3 \cdot {( d ( 1 ) - {d}_{mid} )}^{2} & 2 \cdot ( d ( 2 ) - {d}_{mid} ) & 1 & 0\end{matrix} ) ( \begin{matrix}{A}_{1} \\ {A}_{2} \\ {A}_{3} \\ {A}_{4}\end{matrix} ) ,$$

where r(1), r(2), S(1), S(2) are the values from the table that correspond to lines d(1) and d(2).

TABLE 3
Basis Data for FIG. 12

| | | |
|---|---|---|
| d [m] | r [m] | S [m/m] |

Cone profile 1

| | | |
|---|---|---|
| 0 | 0.034 | 0 |
| 0.025 | 0.0226 | −1.033 |
| 0.059 | 0.0071 | −1.652 |
| 0.093 | 0.005 | 0 |

Cone profile 2

| | | |
|---|---|---|
| 0 | 0.025 | 0 |
| 0.041 | 0.0126 | −0.4326 |
| 0.067 | 0.0066 | −0.0597 |
| 0.093 | 0.006 | 0 |

It is understood that the two-dimensional profiles shown in FIG. 12 are symmetrically rotated about the length (d-axis) to define the shape of the body 4, and that these shapes do not include additional structure of the body 4 such as mounting structures (e.g., flange 5). It is also understood that the outer surface of the body 4 may be dimensioned using a constant wall thickness (e.g., 2 mm or other thickness described herein) based on the internal wall shape.

The body 4 corresponding to Profile 1 in FIG. 12 has a volume of 108,650 mm³ or 108.650 mL. The volume of the chamber produced by Profile 2 in FIG. 12 has a smaller volume, which presents some advantages. For example, the chamber 6 empties more quickly, which assists in flushing gas out of the chamber 6. Additionally, if the transducer 22 is operated in a pulsing manner as described herein, and the time between pulses is configured to flush bubbles out of the chamber 6 and nozzle 14 as described herein, then the use of a smaller chamber 6 means that bubbles can be more rapidly cleared from the propagation path between the transducer 22 and the target. This facilitates complete flushing of the bubbles in the off-time between pulses if the volumes of the chamber 6 and the nozzle 14 are smaller.

In another embodiment, longer off-times for the transducer 22 may be used to clear a chamber 6 and a nozzle 14 having larger volumes. The use of longer off-times, however, results in the time-averaged acoustic power (time averaged over the pulses plus their off-times) that is received by the target decreasing in proportion with the duty cycle (which is defined as the ratio of the on-time to the sum of the off-time and the on-time). Of course, the decision on the length of off-times and on-times during pulsing is a compromise to optimize many factors, including avoiding instabilities on the wall of the stream, avoiding spitting, avoiding reducing the time-average acoustic power delivered to the target to be cleaned to such an extent that it compromises the cleaning, avoiding transducer over-heating, etc. Nevertheless, having a smaller chamber volume produces greater tolerance in using the off-time to clean acoustically-attenuating bubbles from the propagation path between transducer and target, and so eases that optimization. It is understood that the reduction in chamber volume cannot continue indefinitely without compromising the ability of the conical profile of the body 4 to shape the liquid flow and sound field in the body 4 to sufficiently empower the sound field in the nozzle 14 and the stream so that the acoustic energy reaches the target to be cleaned. One consideration in operation is the generation of sufficient acoustic intensity (power divided by area perpendicular to the direction of power transmission) in the nozzle 14 and the shaping of the acoustic mode in the chamber 6 supports this. Generating sufficient intensity in the nozzle 14 where the stream exits generally requires less acoustic power to be emitted from the transducer 22 for Profile 2 than it would for Profile 1. This is because the acoustic power density (power divided by liquid volume) is larger in Profile 2 if the transducer 22 emits the same power with Profile 1. Accordingly, the desired intensity where the stream exits the nozzle 14 can be produced using less electrical power with Profile 2. This is a simplified explanation that ignores modes or resonances and assumes that the same amount of power is exiting into the stream and is lost due to absorption for this comparison, and it is noted that even if the transducer 22 is supplied with the same power, that does not mean the same power is radiated into the volume.

The profiles shown in FIG. 12 and described herein may be particularly suitable for use in a conical body 4 produced from a plastic material, e.g., acrylonitrile butadiene styrene (ABS), with either a solid or partly solid infill material to provide structural stability, and may be manufactured using 3D printing. If the body 4 is 3D printed, it must be made water tight, e.g., by sealing the internal layer of the cone, which (for an ABS material) can be done by painting on acetone to melt the printed layers together. The internal curve of the body 4 should be smooth to reduce nucleation and adherence of air bubbles. In another embodiment, the structure of the body 4 and the operation of the transducer 22 may be configured to produce coupled acoustic modes between the solid wall 10 of the body 4 and the liquid, which in turn couple to coupled acoustic modes between the solid wall of the nozzle 14 and the liquid. This configuration benefits from materials with less dissipation of the acoustic energy, such as metal or glass, although some plastics could potentially be used. It is understood that the body 4 may be made from multiple materials and/or multiple pieces in some embodiments.

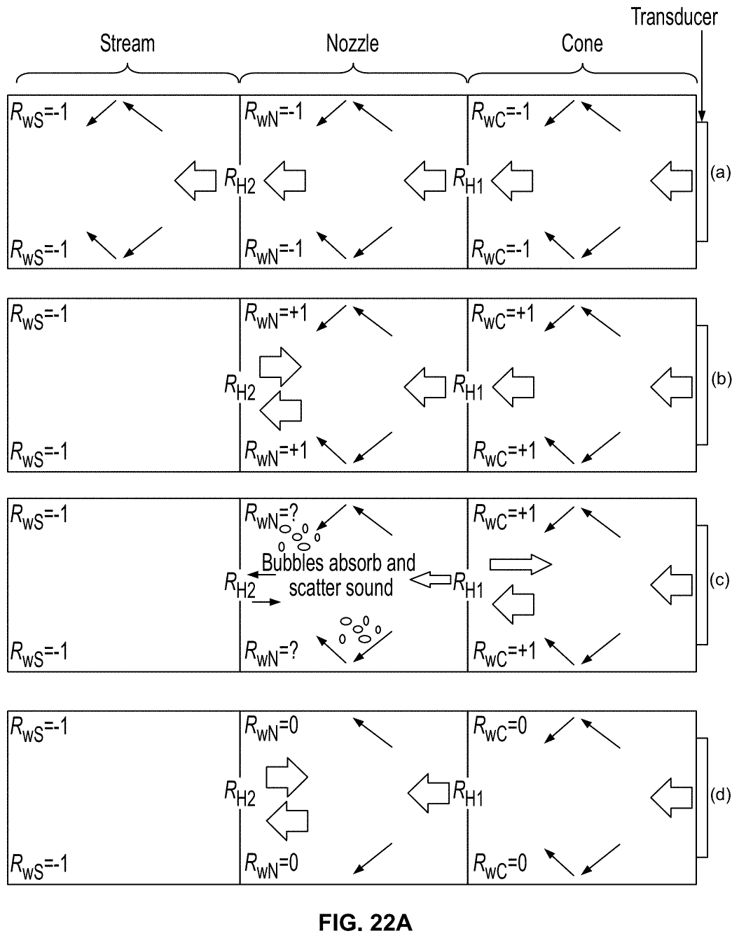

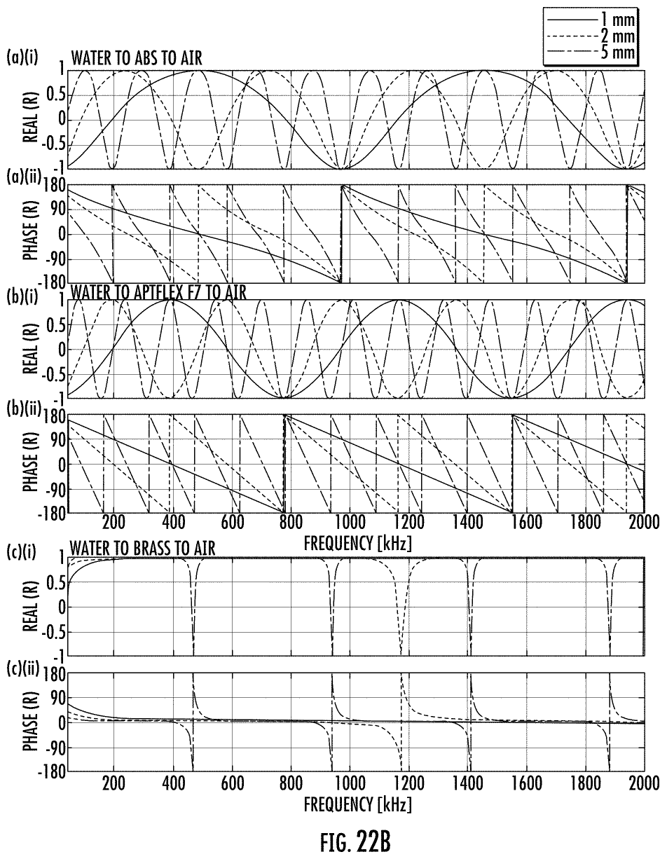

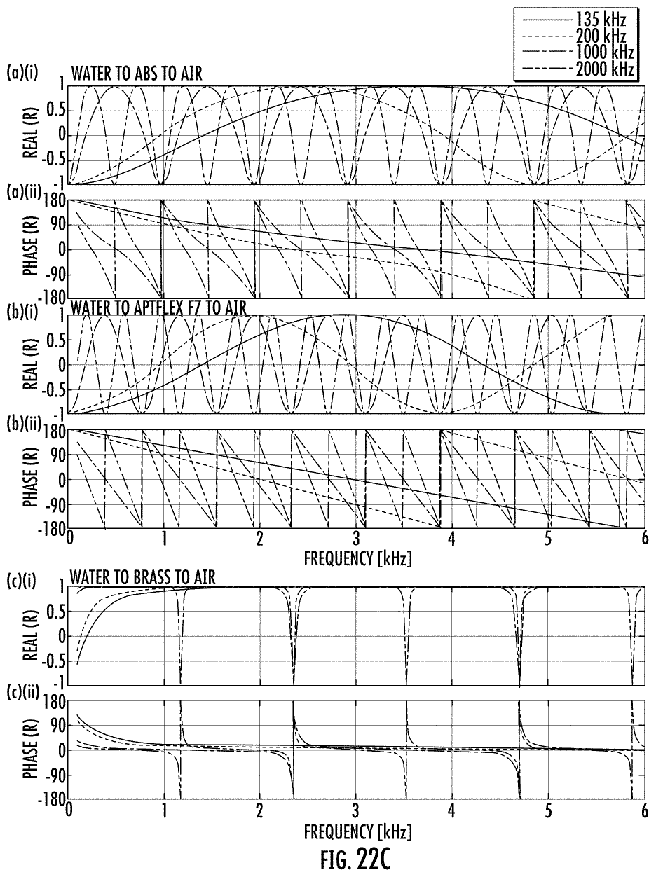

In some embodiments, the structure of the body 4 and the acoustic material properties are such that the transmission of acoustic energy between the nozzle 14 and the stream are maximized. There are a number of ways of accomplishing this, and they rely on the acoustic boundary condition at the interface between different media (the liquid, the solid of the walls of the cone and nozzle, and the air), the differing acoustic impedances of these media, and the way that they couple or fail to couple. The starting point is that, as seen from the acoustic field in the stream as it passes through air, there is an acoustically pressure-release boundary at the interface between the liquid and the surrounding air, represented by the horizontal boundaries in the stream portion at the top and bottom of each panel in FIG. 22A. This is represented in FIG. 22A by the acoustic pressure reflection coefficient R_{wS}=−1, which strongly influences the modal shape of the sound field within the stream. FIG. 22A illustrates how propagation of acoustic waves through the liquid is affected by factors such mismatched or improperly selected reflection coefficients or unwanted bubbles in the stream 7. If the sound field in the nozzle 14 does not match to this in some way, the “transmission line” (extending from right to left in FIG. 22A) between the transducer to the body 4 and the liquid in the chamber 6, to the nozzle 14 and the liquid therein, and then to the stream, will be partially interrupted by junctions between these items. These junctions are represented by the vertical interfaces between the different sections, with the cone/body 4 on the right, the nozzle 14 in the middle, and stream on the left. It is noted that in reality, the junction can be physically distributed over a finite volume between the stream and nozzle, and the nozzle and body 4.

In one embodiment, the frequency of the acoustic energy may be configured, along with the selection of material and structure of the body 4 and/or the nozzle 14, to produce acoustic mode coupling between the liquid and the body 4 and/or the nozzle 14. In other words, the frequency of the acoustic energy may be configured to match a natural frequency of the body 4 and/or the nozzle. Coupled modes between the liquid and the solid material of the body 4 and the nozzle 14 (and, in some circumstances, the target surface), aid in the transmission of acoustic energy from the nozzle 14 to the stream, such that achieving an acoustically pressure-release boundary in the body 4 or in the nozzle 14 as described herein is not necessary. The wave-speed and shape of acoustic waves at the cylindrical section at the end of the nozzle 14 have a different phase velocity than those in a thick-walled stream. The phase-velocity is dependent on the geometry and the material properties of the wall 10. Tuning the mode shape and phase velocity allows a higher transmission efficiency between the nozzle 14 and the stream by acoustically matching the mode shape in the liquid without producing a pressure-release boundary in the nozzle 14. Changes to the body 4 and nozzle 14 cannot change the physical limitations to acoustic propagation (e.g., cut-off frequencies, evanescent pressure fields, etc.) that are imposed by the existence of any pressure release liquid-to-gas perimeter of the stream on acoustic propagation in the stream 7, but can improve the transfer efficiency of acoustic energy from the apparatus 2 to the stream 7. However, if the curved perimeter of the stream is bounded by a solid material (such as a pipe) instead of by gas, then an appropriate choice of the material and thickness of the pipe wall (i.e., matching those of the nozzle) can enable the coupled mode to transmit along the stream as described herein. If the stream is partially bounded by a solid (e.g., the liquid flows over a plate), then mode coupling can still be achieved to allow acoustic energy to propagate further along the liquid/plate interface than it might otherwise do if no coupling occurred. Advantageous materials for use in constructing the nozzle 14 and the body 4 for the transmission and propagation of acoustic energy using this coupled-wave method may be highly resonant with minimal acoustic damping properties, such as metal, glass, and similar materials. This has also been successfully achieved with materials containing more damping such as ABS plastic.

The stimulation of waves with frequencies coupled between the liquid and solid walls (of the body 4 and the nozzle 14) enables the acoustic energy to be effectively transmitted down the stream of liquid, even despite factors that would disrupt or dissipate the acoustic energy. As the acoustic energy is present in both the stream and the wall 10 of the body 4 and/or nozzle 14, the energy is less susceptible to attenuation or disruption due to unwanted bubbles in the body 4 and/or nozzle 14. The use of coupled modes to transfer acoustic energy to the stream from the nozzle 14 benefits from connection of the nozzle 14 to the body 4 in a manner that permits propagation of acoustic energy from the body 4 to the nozzle 14, referred to herein as being “acoustically coupled.” In order to accomplish acoustic coupling, the nozzle 14 and the body 4 may be formed of materials with similar acoustic resonance properties, such as being formed of similar or identical materials and having similar or identical thicknesses. In one embodiment, the nozzle 14 may be formed with the body 4 as a single piece of the same material and thickness.

FIG. 22A shows some ways in which choice of materials for construction of the body 4 allows the efficient transmission of acoustic energy down the transmission line, from transducer through cone through nozzle to stream, by minimizing the horizontal reflection coefficients R_{H1} and R_{H2}: the closer they are to zero, the less energy is reflected back in the opposite direction to that which the transmission line is intended to operate. If R_{H1} is non-zero, some acoustic energy is reflected back into the body 4 and into the liquid therein from the junction between the body 4 and the nozzle 14. This is unwanted as it attenuates the acoustic energy that enters the stream, and also changes the loading on the transducer 22, affecting any tuning done at the transducer to increase electromechanical efficiency. If R_{H2} is non-zero, some acoustic energy is reflected back into the nozzle 14 and into the liquid therein from the junction between the nozzle 14 and the stream. This is also unwanted as it attenuates the acoustic energy that enters the stream.

The easiest way to minimize R_{H2} is shown in panel (a) of FIG. 22A, and involves making the dominant boundary condition at the edge of the sound field (the “acoustic perimeter”) an acoustically pressure-release boundary condition in the nozzle 14 (i.e., R_{wN}=−1). This enables the acoustic mode in the nozzle to match the acoustic mode in the stream, where R_{wS}=−1, the condition that defines what happens upstream. Achieving this condition can be done by making the walls of the nozzle 14 from a material that has a specific acoustic impedance that is very much less than the specific acoustic impedance of the liquid, resulting in a strong reflection occurring at the interface between the liquid and the wall 10 of the body 4, the phase of the pressure wave in the liquid being inverted by the reflection. This would be the case if the walls were made of a material having a specific acoustic impedance that is much less than that of the liquid, such as an expanded foam (e.g., expanded polystyrene). An example of this configuration is shown in FIG. 19, where the walls of body 4 and the nozzle 14 (or at least the inward facing surfaces thereof) are made from expanded polystyrene, and the acoustically pressure-release boundary is located at the interface between the liquid and the walls. If this configuration is used, then it is necessary to ensure that this does not make R_{H1} large, which can be accomplished by making the walls of the body 4 out of a material that has the same acoustic property as the material in the walls of the cone, so that R_{wC}=−1, as also shown in panel (a) of FIG. 22A. In this configuration, there is a strong, phase-inverted reflection of the pressure wave where the sound field in the liquid meets the wall, creating an acoustically pressure-release interface in both the nozzle 14 and the body 4, where they meet the liquid. The acoustic perimeter therefore occurs where the liquid meets the wall, because almost no acoustic energy propagates out from the liquid beyond that boundary. As shown in panel (a) of FIG. 22A, if all these conditions are met, then the transmission line efficiently transmits the acoustic intensity (as shown by the arrows in FIG. 22A) from the transducer 22 and into the stream. It is noted that the acoustic pressure amplitude at the acoustic perimeter is zero if the acoustic pressure reflection coefficient for the sound field contained within that perimeter is −1 (i.e., the acoustic perimeter is acoustically pressure-release to the sound field within the acoustic perimeter). This is because the acoustic pressure wave reflected from that perimeter is phase-inverted, and in antiphase to the pressure wave that is incident on the perimeter from within it, such that the two cancel each other out on the perimeter. In one embodiment, a body 4 shaped as shown in FIGS. 1-4 or as shown in FIG. 12 may be made from expanded polystyrene or another material with similar properties, as discussed above. Such a material may have properties listed in Table 4 below, or properties within 90-120% of these values or 95-105% of these values in other embodiments:

TABLE 4
Typical material property values for Expanded Polystyrene Foam

| Property | Value |
|---|---|
| Density (kg/m³) | 11-32 or 13-17 |
| Young's Modulus | 11.0-40.9 MPa |
| Poisson's Ratio | 0.35 |
| Acoustic Impedance | Unknown at present but expected to be one or two |
| | orders of magnitude less than that of water |

Another embodiment gives an alternative way to achieve the condition shown in panel (a) of FIG. 22A, specifically by making the walls of the nozzle 14 and the body 4 out of a material that has a similar specific acoustic impedance to the liquid, and also does not significantly absorb the acoustic energy over the distance through the body 4 and the nozzle 14, out to the interface between that wall and the surrounding air (the acoustically-pressure-release interface), and back to through the wall to re-enter the liquid. If the conical body 4 and/or the nozzle 14 has a perimeter (the outer wall of these structures) from which acoustical signals in the liquid are reflected with a pressure amplitude reflection coefficient (R) close to −1, −0.95 to −1.0, preferably from −0.99 to −1.0, almost all the incident energy is reflected back into the liquid, with a 180 degree phase change occurring in the pressure waveform on reflection. The acoustic perimeter, where a strong phase-inverted reflection of the acoustic pressure-wave occurs, occurs between the wall and the surrounding atmosphere in this embodiment. This can be achieved in one embodiment by using a body 4 shaped as shown in FIGS. 1-4 or as in FIG. 12 made from Aptflex F7 rubber or another material with similar properties, such as a material with properties listed in Table 5 below, or properties within 90-120% of these values or 95-105% of these values in other embodiments:

TABLE 5
Typical material property values for Aptflex F7 rubber

| Property | Value | |
|---|---|---|
| Density | 0.965 | g/cm³ |
| Bulk Modulus | 1.7 | GPa |
| Poisson's Ratio | 0.48 | |
| Acoustic Impedance | 1.50 | Mrayls |
| Plane wave speed | 1550 | m/s |

| | |
|---|---|
| Shear wave speed | N/A |

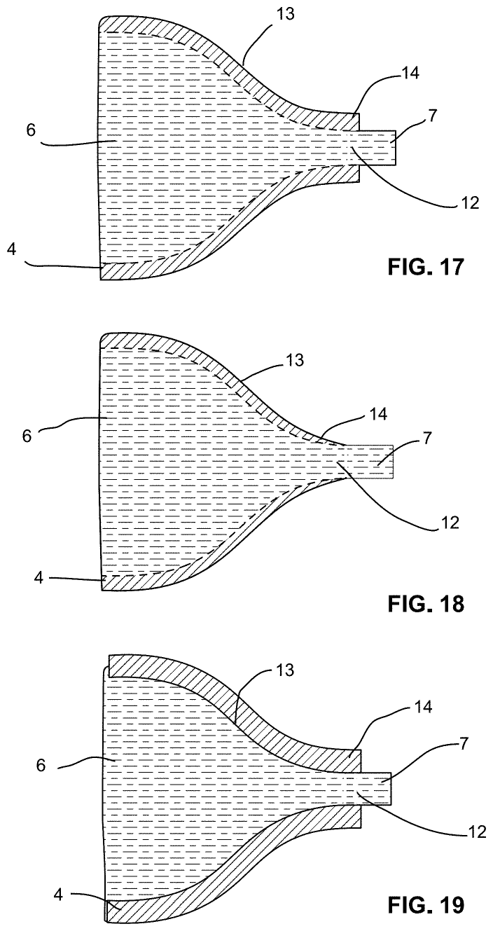

FIG. 17 shows a configuration where the wall 10 of the body 4 and the nozzle 14 is made of such a rubber (commonly-called rho-c rubber) but the thickness of the wall of the nozzle 14 does not taper. This causes some reflection of the energy back up the “transmission line,” because the radius of the acoustic perimeter undertakes a discontinuous narrowing between the nozzle and the stream, which is not ideal. The configuration shown in FIG. 18, where the thickness of the wall of the nozzle 14 tapers toward the end, can significantly address this issue. In the configuration shown in FIG. 18, the radius of the acoustic perimeter continuously narrows from the body 4 to the stream, without sharp discontinuity.

These embodiments, therefore, show two ways by which the walls of the conical body 4 and the nozzle 14 can be composed of a material and structurally configured to achieve a pressure-release condition at a desired point at the nozzle 14 between and including the inner and outer boundaries of the nozzle 14 walls as experienced by the acoustic field in the liquid. FIGS. 17-19 illustrate different pressure-release boundaries for the body 4 and the nozzle 14 based on the material(s) and structures of the same, as described herein.

If the walls of the nozzle 14 are made from, or lined with, a material that absorbs the sound, then the acoustic field in the liquid experiences an acoustic pressure reflection coefficient of R_{wN}=0, and the acoustic pressure amplitude of the acoustic field at the interface between the liquid and the absorbing wall is finite. This is the case shown in panel (d) of FIG. 22A, where a horizontal arrow to the right indicates reflection of energy back up the “transmission line” toward the direction of the body 4 and the transducer 22 by a finite R_{H2}. This hinders the device from working even if the walls of the body 4 are lined with, or made from, absorbing material to ensure an acoustical match between the energy in the body 4 and the energy in the nozzle 14 contents by minimizing R_{H1}.

If the walls of the body 4 and the nozzle 14 form acoustically rigid boundary conditions with the liquid (as given by R_{wC}=+1 and R_{wN}=+1 respectively), then although acoustic energy will pass from the body 4 into the nozzle 14 (as shown in panel (b) in FIG. 22A) because of a near-zero value of R_{H1}, much of this energy will be reflected back at the junction between the nozzle 14 and the body 4. This is because R_{w2} is finite, and this effect is shown by the arrow facing to the right in panel (b) of FIG. 22A. Energy does not efficiently enter the stream in this configuration, which strongly hinders the apparatus 2 from working. It is understood that conditions may differ if coupled modes between the liquid and the walls of the nozzle and cone are used.

The scheme in FIG. 22A of well-defined conditions on the acoustic perimeter of the body 4 becomes complicated when the acoustic boundary condition at the acoustic perimeter is less well-defined. An example of this is shown in panel (c) of FIG. 22A, the example being where bubbles in the liquid in the body 4 (flowing through, or held in place by Bjerknes forces) change the acoustic impedance of the liquid, potentially in a time-varying way. This creates a partial reflection of energy back up the transmission line at the junction between the body 4 and the nozzle 14 through a finite R_{H1}, and another partial reflection of energy back up the transmission line at the junction between the nozzle 14 and the stream through a finite R_{H2}. Another example would be if the body 4 were made of plastic, or a metal that provided a non-rigid interface between the liquid and the walls of the cone and the nozzle 14. In both circumstances, there is acoustic coupling between the liquid and the walls of the body 4 and the nozzle 14, and this coupling allows coupled modes between the liquid and the solid. In this embodiment, a body 4 shaped as shown in FIGS. 1-4 or FIG. 12 may be made from ABS plastic or another material with similar properties, such as a material with properties listed in Table 6 below, or properties within 90-120% of these values or 95-105% of these values in other embodiments:

TABLE 6
Typical material property values for ABS Plastic

| Property | Value | |
|---|---|---|
| Density | 1.07 | g/cm3 |
| Young's Modulus | 2.5 | GPa |
| Poisson's Ratio | 0.35 | |
| Acoustic Impedance | 2.1 | MRayls |
| Plane wave speed | 1940 | m/s |
| Shear wave speed | 970 | m/s |

Alternatively, the walls of the body 4 could be made of a copper alloy such as brass. In this embodiment, a body 4 shaped as shown in FIGS. 1-4 or FIG. 12 may be made from brass or another material with properties listed Table 7 below, or properties within 50-150% of these values or 80-120% of these values in other embodiments. It is understood that some variation occurs around these depending on the type of brass, e.g., cast, 70-30, etc.:

TABLE 7
Typical material property values for Brass

| Property | Value |
|---|---|
| Density | 8.44-8.75 g/cm³ |
| Bulk Modulus | 108 GPa; or 105-115 GPa |
| Poisson's Ratio | 0.331 |
| Acoustic Impedance | 39.7-41.1 MRayls |
| Plane wave speed (m/s) | 4700 |
| Shear wave speed (m/s) | 2110 |

The thickness of wall 10 may be configured to provide a specific pressure release boundary. If the wall 10 is sufficiently thin, and the frequency of the acoustic energy is sufficiently low (while remaining high enough to propagate down the stream), then the air on the side of the wall 10 opposite the liquid enables resembles an acoustically-pressure-release boundary from the perspective of the acoustic field in the liquid. Walls 10 made from denser materials (e.g., steel, brass etc.) require smaller thicknesses to achieve the same effect at the same frequency. If a thicker wall 10 is used, the wall 10, the liquid, and the acoustic frequency can be designed to exploit coupled modes to enable sound to propagate through the body 4, wall 10, and nozzle 14 and into the stream.

Coupled modes (coupling acoustic energy between the liquid and the solid walls) allow the efficient transmission of energy through the nozzle 14 into the stream, despite the existence of the pressure-release boundary at the acoustic perimeter in the stream. To enable coupled modes in the nozzle 14 to transmit acoustic energy into the stream, then the walls of the nozzle 14 need to be acoustically coupled to those of the body 4. For example, a nozzle 14 made of copper water pipe can be very effectively joined to a body 4 made of brass, e.g., by soldering or welding, to efficiently transmit energy from the body 4 into the nozzle 14, and from there into the stream. Any air gaps in that joint should be avoided, as they would hinder this coupled mode operation from working to efficiently transmit energy from the body 4 into the nozzle 14 and then into the stream.

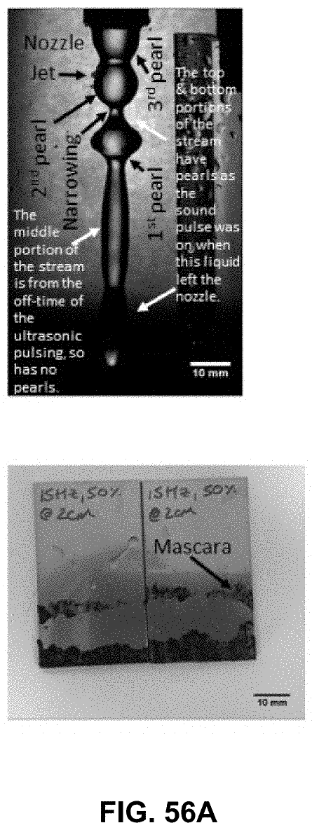

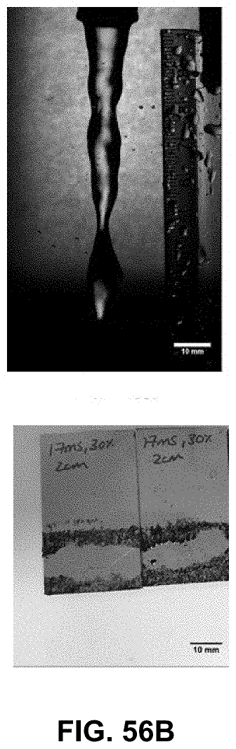

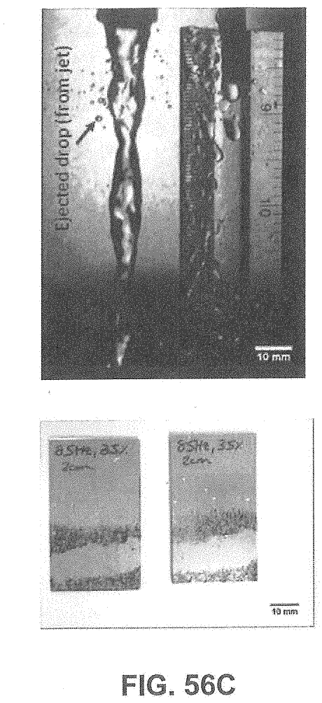

The presence of surface waves in the stream can be problematic, reducing the range to which the ultrasound can propagate down the stream by causing oscillatory narrowing of the stream, to a width that makes the cut-off frequency at that point greater than the acoustic frequency, such that the energy beyond that point is evanescent unless the stream rapidly widens over a sufficiently short distance allow further propagation. Such surface waves can eventually cause stream to break up, and the acoustic energy cannot pass from liquid droplet to liquid droplet through the air because of the strong acoustic mismatch between air (or other atmosphere) and the liquid. The surface waves on the stream are created and driven by the acoustic energy that passes through. This driving occurs over a slower timescale than one acoustic cycle, and so the time-average (averaged over the time-scale of the stream surface wave) power that drives the growth of these waves can be reduced by pulsing the sound field over timescales that are significantly shorter than the timescale by which the stream surface wave grows, but longer then the timescale of one acoustic cycle. This is described herein with respect to FIGS. 56A-C. The growth of surface wave on the stream can also be reduced by increasing the dissipation at the source, i.e., where the stream exits the nozzle 14, by the use of materials in or attached to the nozzle 14 wall that move as the surface stream tries to form and so dissipate energy from it, reducing its ability to grow. The use of materials as specified above can assist in damping and reducing instabilities on the surface of the stream, thereby resisting breakup of the stream as described herein. Furthermore, appropriate design can generate surface waves in the material of the body 4 and especially the nozzle 14. The body 4 can thereby transmit acoustic waves to the nozzle 14 that are imparted to the stream 7 to damp and/or counteract surface instabilities in the stream 7.

FIGS. 22B and 22C illustrate how achieving a pressure-release condition is influenced by material selection, thickness of the wall 10, and frequency of the acoustic energy. FIGS. 22B and 22C represent a simplified explanation of the complicated acoustic interactions at the boundary between the liquid and the wall of the cone, and between the liquid and the wall of the nozzle, but are illustrative of some of the phenomena occurring at these boundaries. The simulations illustrated in FIGS. 22B-C represent a simple three-medium situation, where the three media meet at planar, flat interfaces, where acoustic plane waves are projected towards this set of three media, and where the waves come from the first layer, which is a semi-infinite layer of water in this simulation. All of these criteria are used as approximations, because the liquid in the chamber 6 is not semi-infinite, the acoustic waves that approach the wall 10 are not all planar and are not all normally incident against the wall 10, and the wall 10 is not flat or planar. The “inner interface” is the boundary between the liquid (in this simulation, water) and the wall 10, which in the simulations of FIGS. 22B-C is made of (a) ABS plastic, (b) acoustic polyurethane rubber (e.g., Aptflex F7, Precision Acoustics Ltd, UK) and (c) brass. The sound that penetrates the material of the wall 10 in these simulations travels through the wall 10, to reflect off the “outer interface” between the solid and the surrounding air. This reflection launches a reflected wave, which propagates back through the solid and crosses the “inner interface” to enter into the liquid. For each material, FIGS. 22B-C plot the real part of the reflection coefficient (the ratio of the complex number representation of the reflected pressure wave to that of the incoming pressure wave), and the phase of the reflected wave at the moment it crosses the “inner interface.” FIGS. 22B-C illustrate when the phase of the reflected wave on the “inner interface” is 180° (or −180°), which is the desired phase or approximate phase. If the profile of the “inner interface” matches the outer perimeter of the stream when it exits the nozzle, then the acoustic boundary is matched smoothly for the mode that is desired to result in the stream. It is noted that this simulation has other limitations in addition to the above assumptions, in that it fails to account for the shear wave in the wall material or to account for any absorption.

FIG. 22B plots the real part (represented in part (i) of each plot) and phase (represented in part (ii) of each plot) of the reflection coefficient as a function of the ultrasonic frequency for walls of (a) ABS plastic, (b) rubber (Aptflex F7), and (c) brass, each wall having thicknesses of three illustrative examples: 1 mm, 2 mm and 5 mm. The frequency axis extends from less than 100 kHz to up to 2 MHz. FIG. 22C presents the same scenarios, with the continuously-varying parameter being the wall thickness plotted using the same three illustrative example materials and thicknesses, and the discrete parameter being the frequency. While absorption in the walls is not modelled in this simulation, there nevertheless is a sizeable reflected wave. Thus, the phase of the reflected wave (part (ii) of each graph) is a crucial starting point to observe. For ABS and rubber (Aptflex F7), the phase tends to decrease with increasing frequency (the apparent discontinuities simply being phase wrapping displays), starting from +180 degrees at low frequencies. For walls 1 mm thick in FIGS. 22B-C, the phase falls to −180 degrees at just under 800 kHz for rubber (Aptflex F7), as shown in plot b(ii), and at just under 1000 kHz for ABS, as shown in plot a(ii). Given that a pressure-release condition is desirable on the inner interface, to match the modes in the nozzle 14 with the modes in the stream, then a 5 mm thick nozzle wall of Aptflex F7 (and a similar wall in the body 4) would achieve this for a 160 kHz sound field because that is when the phase is close to +/−180 degrees (FIGS. 22B-C, plot b(ii)). The pattern repeats regularly because of the ratio of the acoustic wavelength in the solid and the thickness of the solid itself. However, a 1 mm thick wall of ABS would achieve this at around 960 kHz (FIGS. 22B-C, plot a(ii)). This illustrates that various combinations of wall thickness and frequency, depending on the material, may be better than others at coupling with the modal sound field in the stream to achieve a pressure-release condition on the perimeter of the stream, which is the same perimeter as the inner interface in the nozzle 14. In practice, the resonances shown will have some damping, but the ABS and rubber materials have smooth changes in phase, so damping will have relatively less effect than for a material such as brass, discussed below. By identifying the combinations of frequency, liquid, and solid (and its thickness) that give a phase of close to +/−180 degrees, it is possibly to identify conditions that would allow the nozzle 14 to produce modes in the liquid that couple with those in the stream.

The brass example in FIGS. 22B-C, plot c illustrates the limitations of this simple simulation. As expected for a material with such a high sound speed and high density (both significantly greater than water), the decrease of phase with increasing frequency (FIG. 22B, plot c(i)) is not monotonic as it is for ABS and rubber. The brass mainly produces a zero phase change in the reflected wave at the position of the inner interface, departing from this only for narrow frequency ranges at appropriate ratios of the acoustic wavelength to the wall thickness. From this example, one might therefore infer that brass, or any other metal, would not make a suitable material for the construction of conical bodies to couple sound fields through nozzles into liquid streams. However, the use of a material with acoustic properties such as brass can achieve unexpectedly advantageous results by enabling the use of coupled modes to transmit acoustic energy to the stream as described herein.

It has also been found that the acoustic waves transmitted down the stream stimulates surface instabilities on the wall of the stream. These instabilities may result in “pearling” of the stream 7, which creates narrowed portions of the stream 7 between the “pearls” (globular regions of liquid, attached at two points along the length of the stream, one facing the upstream direction and one facing downstream), such that the existence of these narrowed portions may limit propagation of acoustic waves through the narrowed portions of the stream 7. These instabilities can also contribute to partial break-up of the stream prior to reaching the target, reducing or eliminating the ability of the stream to conduct the acoustic waves. The acoustic energy cannot pass from liquid droplet to liquid droplet through the air because of the strong acoustic mismatch between air (or whichever gas is relevant) and liquid. The stream 7 can also be disrupted by “spitting” from the stream 7, in which liquid droplets are ejected from the surface of the stream 7, which may create aesthetic, cleanliness, or contamination issues. Even though the liquid is clean leaving the nozzle 14, the generation of liquid droplets is unwanted for the aesthetics (particularly as it may be confused with respiratory droplet hazard). Spitting may also create liquid settling on other surfaces, generating hazards such as slipping. Spitting can be reduced by reducing the incidence of bubbles, particularly bubbles larger than resonance size, from leaving the nozzle 14 and entering the stream 7, which can be accomplished by several techniques or combinations thereof. For example, adjusting the pulsing regime, including the use of a smoother, e.g. sinusoidal, envelope of the pulses, as opposed to a square wave, may reduce spitting. Other techniques to reduce spitting include appropriately outgassing the liquid as it enters the chamber 6 and/or reducing the amplitude of the signal from transducer 22 (though not to the degree that it will compromise the ability to clean or treat a surface). Reduction in spitting can also be assisted by reducing pearling using the methods described herein, such as in the previous paragraph.

FIGS. 13-16 illustrate different shapes for the nozzle 14 that may at least partially mitigate this effect. The nozzles 14 in FIGS. 13-16 all have stepped configurations with a plurality of protrusions 15 projecting from the front of the nozzle 14 around the orifice 12, with the protrusions 15 being separated by gaps 17. The protrusions 15 and gaps 17 may be arranged in a repeating pattern around the periphery of the nozzle 14 in these embodiments. In one embodiment, the protrusions 15 may be very small in size and/or may be somewhat flexible, e.g., brush bristles or similar structures. The protrusions 15 may also have a hydrophobic coating on at least the inner surface thereof, to assist in keeping the protrusions 15 at the surface of the stream 7. Movement of these flexible protrusions 15 by the surface waves dissipates the energy of those surface waves close to the nozzle 14, thereby preventing the waves from growing because the rate at which energy is dissipated from them by the work the surface waves do on the flexible structures. Viscothermal losses that ensue from the damped motion of the protrusions 15, outweighs the rate at which energy is supplied to them by the ultrasound field to make the stream surface waves grow. In another embodiment, the protrusions 15 may be larger and more structurally (as opposed to acoustically) rigid, which can additionally or alternately cause phase change at the launch of any instabilities on the wall of the stream, and also of any energy reflected back upstream from the target.

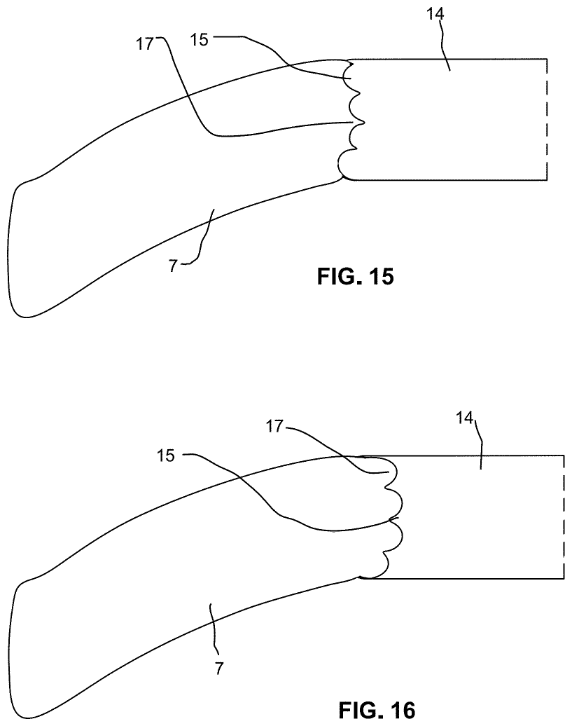

The nozzle 14 in FIG. 13 has a plurality of rectangular protrusions 15 separated by rectangular gaps 17, creating a gear tooth-like configuration. The nozzle 14 in FIG. 14 has a plurality of triangular protrusions 15 separated by triangular gaps 17, creating a jagged configuration. Optional turbulence tamers 38 within the nozzle 14 are shown. When the smallest number of protrusions 15 are used, the shape of the nozzle 14 may resemble that of a hyperemic needle. The nozzle 14 in FIG. 15 has a plurality of rounded protrusions separated by tapered, pointed gaps 17. The nozzle 14 in FIG. 16 has a plurality of tapered or serrated protrusions 15 separated by rounded gaps 17. In these configurations, some portions of the liquid exiting the nozzle 14 contact the protrusions 15 for a longer period of time than the portions of the liquid exiting at the gaps 17. This creates surface waves that have a damping effect on such instabilities, thereby reducing break-up of the stream. If the effect of surface wave energy reflect from the target is small, then if the depth of the protrusions 15 along the stream is half a wavelength of the “pearling” phenomenon on the stream, destructive interference between surface wave components that are excited out of phase on different portions of the stream perimeter will reduce the amplitude of the surface waves. If, however, the growth of surface waves on the stream is significantly enhanced by surface waves reflected back in the upstream direction from a target, then a depth of castellation of a quarter of a wavelength of the “pearling” phenomenon on the stream is preferred. A mix of such depths can be used if the extent of reflection cannot be predicted. It is noted that the wavelength of the surface wave on the stream produced by the instabilities discussed herein can be approximately equal to the wavelength of the ultrasound transmitted by the transducer 22, and so the use of such protrusions 15, and transducer 22 pulsing regimes, to suppress the instabilities is determined by the rise times of these instabilities, their wavelengths and periods. Other configurations of nozzles 14 may produce a similar effect, including other configurations with protrusions 15 and gaps 17.

The transducer 22 is configured to introduce acoustic energy (e.g., in the form of acoustic waves) into the chamber 6, which travels through the chamber 6 and out of the nozzle 14 through the stream to the target. The acoustic energy may have ultrasonic frequencies. One embodiment of a transducer 22 uses one or more piezoelectric elements 50, such as a circular piezo ceramic disc or other piezoelectric disc, polarized (e.g., silvered) for opposite polarity on each of its flat surfaces. In one configuration, the transducer 22 may include a piezoelectric element 50 in the form of a single piezoelectric disc bonded to the rear wall 8 by a suitable adhesive that will not delaminate or fracture or de-bond under expected use. This embodiment may be configured to produce ultrasonic energy with high ultrasonic frequencies greater than 500 kHz. In another configuration, usually used for lower frequencies (with some overlap of these frequency ranges), the transducer 22 may include one or more piezoelectric elements 50 located between a head mass 51 and a tail mass 52, with a tensioning bolt 53 connecting the head mass 51 and the tail mass 52. The piezoelectric elements 50 in this embodiment may be provided in pairs, which aids in grounding of the head and tail masses 51, 52. It is understood that other types of acoustic transducers 22, including those capable of emitting ultrasonic waves, may be used in other embodiments. Additionally, the apparatus 2 may include multiple transducers 22 in one embodiment, which may be used to avoid overheating if the demand on a single transducer 22 could potentially cause overheating.

Table 8 below describes some effective ranges and parameters for general pulsing regimes for the transducer 22. It should be noted that continua between these limits of acoustic carrier frequency and nozzle 14 diameter are permissible and at times desirable. For example, use of an acoustic frequency of 135 kHz places that carrier frequency close to the cut-off frequency for a nozzle diameter of 10 mm. For this nozzle size, switching up to a higher frequency (e.g., 200 kHz) will allow the device to operate while placing more acoustic modes of the stream above the cut-off frequency, allowing them to propagate. Therefore, Table 8 should be seen as a representation that the frequencies stated across the top of the table are the lower bounds of the frequency that can be used for the nozzle 14 of the statement diameter, and that frequencies of up to two or three times this may be appropriate. By widening the nozzle 14, the minimum carrier frequency can be reduced, allowing for a low frequency such as 100 kHz to propagate down a wider stream than the widest shown in the table.

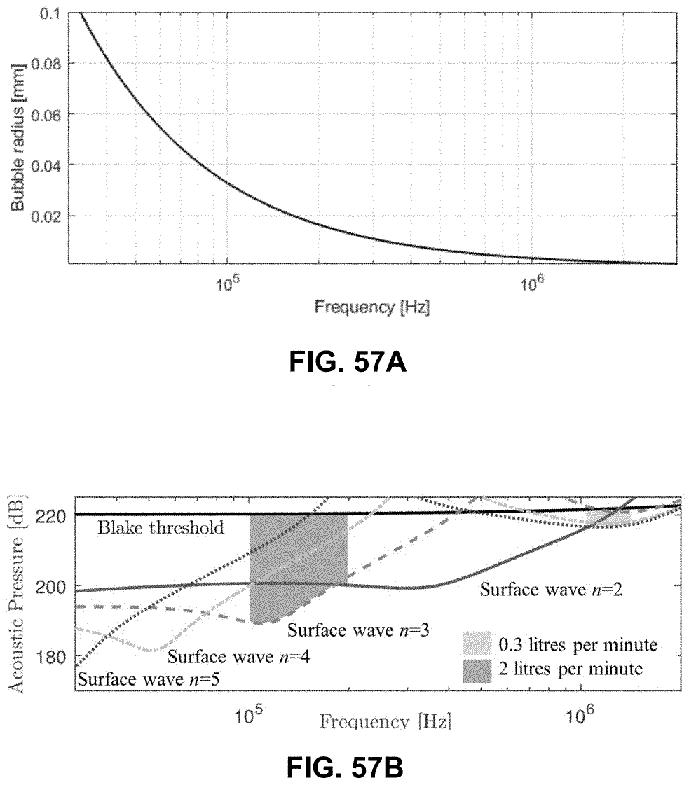

It is noted that the rms acoustic pressure amplitudes in Table 8 are inferred from the calculations in FIG. 57B that show the threshold acoustic pressure (dB re 1 μPa) that must be present at the bubble at the target to stimulate a surface wave of order n (measured at the target). In FIG. 57B, only axisymmetric modes are plotted, and other modes are not shown for clarity. The amplitude of the ultrasound field at the location of the bubble (the target) must exceed the threshold required to excite that spherical harmonic perturbation which has a frequency that is half of the driving ultrasound field. That perturbation, of mode n, which varies with bubble size, is termed the “Faraday wave.” This threshold amplitude is lowest when the bubble is driven at its pulsation resonance frequency f₀ (corresponding to the minimum in the curves shown, one for each mode n in FIG. 57B), which increases with decreasing bubble radius (R₀) in the manner shown in FIG. 57A. At any given frequency, the plot in FIG. 57B considers only bubbles that are pulsation resonant at that frequency. For such bubbles, increasing the acoustic pressure amplitude only excites a pulsation until the first shape mode curve is crossed, which for that bubble will constitute the Faraday wave. As the acoustic pressure is increased further, bubbles of sizes a little larger or smaller than resonance will pulsate to sufficient amplitude to host that Faraday wave, the range of bubble sizes increasing (but still clustered about the pulsation resonance) with increasing acoustic pressure amplitude. As the acoustic pressure increases further, the threshold is passed to excite a second surface wave. Additional higher order modes are excited as the acoustic pressure increases. If the device is intended to avoid inertial cavitation, then the acoustic pressure amplitude at the target at the bubble must be above the Faraday wave threshold, but low enough to not cause inertial cavitation at the cleaning location (i.e., below the Blake threshold). The two shaded areas approximately show frequency ranges that would function well for embodiments of apparatuses 2 that run at nozzle 14 flow rates of 2 liters per minute and 0.3 liters per minute. This is determined by the fact that these flow rates suggest nozzle diameters of 10 mm and 2 mm, respectively, to obtain these liquid flow speeds. The liquid flow speeds, in turn, determine the use of frequencies (Table 8) of approximately 135-250 kHz, and approximately 1-3 MHz, respectively, which determine use of the shaded areas in FIG. 57B, and hence the rms acoustic pressure amplitude ranges given in Table 8. It is noted that the parameters in Table 8 are effective, but not exclusive, pulsing regimes for three example acoustic carrier wave frequencies.

TABLE 8

| | | | |
|---|---|---|---|
| Minimum | 135 kHz | 700 kHz | 1 MHz |
| Carrier | | | |
| Frequency | | | |
| Nozzle | 10 mm + 30% | 4 mm + 20% | 2 mm + 20% |
| diameter | | | |
| Pulse | 0 Hz (i.e. continuous- | 0 Hz (i.e. continuous- | 0 Hz (i.e. continuous- |
| Repetition | wave) to 100 Hz ± 50% | wave) to 300 Hz ± 50% | wave) to 800 Hz ± 50% |
| Frequency | | | |
| (PRF) | | | |
| Alt. range 1 | 0 Hz (i.e. continuous- | 0 Hz (i.e. continuous- | 0 Hz (i.e. continuous- |
| | wave) to 50 Hz ± 50% | wave) to 100 Hz ± 50% | wave) to 200 Hz ± 50% |
| Alt. range 2 | 0 Hz (i.e. continuous- | 0 Hz (i.e. continuous- | 0 Hz (i.e. continuous- |
| | wave) to 25 Hz ± 50% | wave) to 50 Hz ± 50% | wave) to 75 Hz ± 50% |
| Duty cycle | Continuous-wave to 55% | Continuous-wave to 55% | Continuous-wave to 55% |
| On time | 0.01 ms to continuous- | 0.005 ms to continuous- | 0.001 ms to continuous- |
| | wave | wave | wave |
| Alt. range 1 | 0.05 ms to continuous- | 0.03 ms to continuous- | 0.01 ms to continuous- |
| | wave | wave | wave |
| Alt. range 2 | 0.1 ms to continuous- | 0.1 ms to continuous- | 0.1 ms to continuous- |
| | wave | wave | wave |
| Off time | 0 (i.e. continuous-wave) | 0 (i.e. continuous-wave) | 0 (i.e. continuous-wave) |
| | to 500 ms, preferably 70- | to 500 ms, preferably 70- | to 500 ms, preferably 70- |
| | 200 ms | 200 ms | 200 ms |
| Rms | 4.1 kPa-103.5 kPa | 30.9 kPa-113.5 kPa | 64.6 kPa-118.9 kPa |
| acoustic | | | |
| pressure | | | |
| amplitude | | | |

In the embodiment of FIGS. 1-11, the apparatus 2 is configured such that the acoustic energy emitted by the transducer 22 generates bubbles within the body 4 via cavitation (both inertial and non-inertial, e.g., Rectified diffusion). If the acoustic energy is of a sufficiently high amplitude and the on-time (tₒₙ) of the transducer 22 is above a threshold (e.g., 67 μs) bubbles will be generated throughout the body 4 at acoustic antinodes due to the formation of standing waves in the cone. Additionally, if the bubbles are in the sound field for a sufficient time, ultrasonic degassing of the liquid will occur at these locations. Such bubbles will build up and potentially adhere to the body 4, and potentially to the rear wall 8 and the manifold 40, during high amplitude acoustic energy generation by the transducer 22. More specifically, when acoustic energy is introduced into the chamber 6, Bjerknes forces cause bubbles to congregate at acoustic pressure nodes and antinodes. Such bubbles can attenuate the sound field from reaching the stream, which is undesirable. When the transducer 22 is turned off, production of such bubbles will decrease or stop, and the bubbles are no longer held to the acoustic pressure nodes/antinodes and can travel with the liquid flow out of the nozzle 14. Thus, pulsed activation by the transducer 22 can be used to sequentially generate and release bubbles that will travel through the stream 7 to the target for activation.

The pulsing frequency and/or the off-time (time between pulses) of the transducer 22 can be configured so that the acoustic waves reach the target at the same time as the bubbles released when the transducer is turned off reach the target, considering the relative speeds at which the acoustic waves and bubbles travel through the liquid. In general, during the off-time of the pulse, the bubbles travel through the chamber 6 and the stream 7 at the local velocity of the liquid (with usually small corrections for buoyancy). The pulsing frequency and/or the off-time can also be configured to permit all generated bubbles to clear the chamber 6 before the transducer 22 is switched back on. Additionally, the amplitude of the acoustic energy generated by the transducer 22 is configured to produce predominantly resonant bubbles (based on the frequency of the energy) and to minimize or avoid production of unwanted bubbles. In general, changing the operating parameters (e.g., amplitude, frequency, tₒₙ) of the transducer 22 can produce larger or smaller bubbles, respectively. Further, the on-time of the transducer 22 is configured to produce a desirable number of bubbles, i.e., to produce bubbles in a sufficient number to achieve beneficial effects on the target but not so high as to create agglomerations that may disrupt passage of acoustic waves through the liquid. The non-ideal nature of this set-up is that (depending on the volumes of the body 4 and the nozzle 14 and the flow speed) off-times of several seconds may be needed to completely replace the bubbly liquid in the body 4 and the nozzle 14 with fresh liquid that does not contain unwanted bubbles, and long off-times reduce the proportion of time that the stream is actively cleaning or treating the target. To mitigate this, the off-time can be reduced so that it clears a sufficient proportion of bubbles out of the propagation path, notably out of the nozzle 14 where the flow is fastest, allowing others from within the cone to be removed when they, at the cessation of the Bjerknes forces when the on-time ends, are free to be captured by the flow into the vent 9. Bubble generation in this way, using the transducer 22 both to generate the bubbles and to provide the pulse that activates the bubbles on the target to undertake cleaning and treatment of the target through surface waves on the bubble wall, can use either the same pulse to the transducer 22 to achieve both tasks, or alternate pulses. In one embodiment, alternate pulses may include a shorter, higher amplitude pulse (for bubble generation) between each longer, lower-amplitude pulse that cleans or treats the target. Alternatively, a bubble generation system (e.g., another transducer, a bubble injection, venturi or electrolysis system) may be placed close to the point where the stream exits the nozzle, permitting shorter off-times to clear the bubbles from the propagation path.

Provided they do not cause detrimental effects, faster flow speeds and weaker acoustic fields are generally beneficial in reducing the tendency of large bubbles to be trapped in the chamber 6, because faster flows remove bubbles from the chamber 6 more quickly during the pulsing off-time, and when the transducer 22 is on, the flow is more easily able to overcome the tendency of the acoustic field to hold bubbles at the acoustic pressure antinodes (for bubbles smaller than the pulsation resonance size) and nodes (for bubbles larger than the pulsation resonance size), and so achieve removal of the bubbles from the chamber by flow. These parameters must be balanced against other considerations. If the acoustic field is made too weak, then sufficient surface waves are not stimulated on the bubble wall (which is a threshold phenomenon) when the bubbles are at the target. If the flow is too strong, turbulence and circulation in the chamber 6 may disturb the laminar flow too much, increasing the residence time, or even trapping bubbles in the chamber 6. Conversely, if the transducer 22 is driven very strongly to try to generate an intense acoustic field in the chamber 6, it may generate too much cavitation in the liquid in front of the transducer 22, preventing the ultrasound from propagating away from the transducer 22. Such a strongly-driven transducer might also cause too much previously dissolved gas to come out of solution through rectified diffusion or inertial cavitation, generating too many bubbles in the chamber 6 and hence too much attenuation of the acoustic field. Also, if the flow speed from the nozzle 14 is too slow, then the proportion by which the liquid exiting the nozzle 14 narrows as it falls increases, which results in thinning of the stream 7 as it falls, which will eventually prevent the acoustic waves from propagating any further down the stream 7 and reaching the target. It will also cause the stream to break into drops closer to the target, preventing the ultrasound from reaching the target. Conversely, if the flow speed from the nozzle is too fast, it will entrain air pockets in the stream and so attenuate the sound propagation through the target, and generate spray (wetting non-target areas with droplets and potentially creating hazard, whilst wasting water) and potentially damage the target.

The pulsing regime can also be configured to control instabilities in the stream and reduce the break-up of the stream 7, which can prevent propagation of the acoustic energy beyond the narrowing of the stream between the “pearls” generated on it by the instabilities. When the pulse begins, acoustic energy is pumped into the stream, entering it from the nozzle 14, and reflecting back from the target, or from the point at which the stream narrows to make the ultrasonic frequency less than the cut-off frequency. This energy stimulates the growth of these instabilities, but at a finite rate. A sufficiently short on-time means the instabilities do not grow large enough to generate excessive stream narrowing. A sufficiently long off-time between pulses provides time for the excitation of instabilities at the nozzle 14 to damp out, suppressing the amplitude of the instabilities, while the pearls that were fully-formed when the acoustic energy was on travel down the stream away from the nozzle 14, leaving stream without pearls behind them because the transducer 22 was off when that region of stream left the nozzle (FIG. 56A). When the next acoustic pulse starts, the acoustic energy starts forming pearls as these leave the nozzle 14 (in FIG. 56A, the first, second and third pearls formed are labelled). Pearls are unwanted. Short on-times reduce pearl growth, and long off-times allow existing pearls to travel away from the nozzle, but reduce the acoustic energy delivered over time to the target, so a compromise must be reached. It is also understood that flow speed through the nozzle 14 may affect the incidence of such instabilities, and control of the flow speed may assist in reducing instabilities. There is a threshold amplitude for the acoustic generation of pearling, and reducing the amplitude of the acoustic energy to below that threshold will be beneficial if this can be done whilst also achieving cleaning, e.g., by using other measures mentioned herein to reduce the attenuation of the sound between the transducer and the target.

As one example, consider a nozzle resembling (but not identical) to that of the lower of the three frequencies shown in Table 8. In this case, the insonification frequency is slightly lower, 132.2 kHz and the inner diameter of the nozzle is 12 mm. For a stream that leaves such a nozzle 14 made of ABS in this example, the 132.2 kHz carrier frequency could be chosen with a 59 Hz pulse frequency, a 25% duty cycle, a 4.25 ms on-time, and a 12.75 off-time. The on-time may be varied by ±25% in use, and the 25% duty cycle may be maintained by varying the off-time. Long off-times can be associated with placement of a target further down the stream to allow more of the propagation path to be cleared of bubbles through the flushing effect of the water flow during the off-time and to allow pearls to propagate away from the nozzle 14, leaving a longer length of stream at the nozzle 14 that does not contain pearls. As noted herein, during the on-time, such bubbles are held in place by Bjerknes forces or continually generated if inertial cavitation or ultrasonic degassing occurs in the liquid in the body 4, e.g., close to the transducer 22. To ensure cleaning in this regime, the pulse average power (the power measured over a time window that encompasses the pulse on-time but not the off-time) and/or the duration of the on-time may need to be increased. For example, in one experiment, use of pulse of 85 Hz with 35% duty cycle, a 4.1 ms on-time, and a 7.66 ms off-time, reduced stream instabilities while increasing the observed cleaning of a target material placed in the stream, as discussed herein with respect to FIGS. 56A-C. FIGS. 56A-C, discussed in greater detail below, illustrate stream characteristics and cleaning performances for various pulsing regimes.

For a nozzle of 12 mm inner diameter an using an ABS cone and nozzle 14, FIGS. 56A-C illustrate various stream characteristics and cleaning performances for removal of waterproof mascara from stainless steel tiles by cleaning for 5 seconds at 2 cm from the nozzle exit, resulting from various pulsing and duty cycle combinations, with a carrier frequency of 132.2 kHz. The upper and lower panels of FIG. 56A are taken with a 15 Hz pulse repetition frequency (PRF) and a 50% duty cycle (33.3 ms on-time and 33.3 ms off-time), which achieved a 12 mm maximum cleaning diameter. The upper and lower panels of FIG. 56C are taken with an 85 Hz PRF and a 35% duty cycle (4.1 ms on-time and 7.66 ms off-time), which achieved a 12 mm maximum cleaning diameter. The upper panel (the stream image) of FIG. 56B is taken with a 59 Hz PRF and a 12% duty cycle (2.0 ms on-time and 15 ms off-time); however, the lower panel of FIG. 56B is taken with different conditions, using a 59 Hz PRF and a 30% duty cycle (5.1 ms on-time and 11.9 ms off-time), which achieved a 10 mm maximum cleaning diameter. For a given rms acoustic pressure amplitude delivered to the target, if no enhancement was seen by varying the on-time and off-time of pulsing, one would expect to see greatest cleaning (or other surface treatment, all measured over several or more seconds) for continuous-wave ultrasound, because there are no off-times in which there is no acoustic energy delivered to the target. Conversely, when the sound is pulsed, one would expect to see the degree of surface treatment being proportional to the duty cycle, because for a fixed rms acoustic pressure at the target, the acoustic energy delivered to the target over several seconds is proportional to the duty cycle, all other factors staying the same. However, these results are not seen in comparing FIG. 56A with 56C, where a reduction in duty cycle does not lead to a corresponding reduction in cleaning. Surprisingly, the same cleaning is maintained using less ultrasonic energy over several seconds, proving that some combinations of on-time and off-time lead to an enhancement in the efficiency of surface treatment. The 50% duty cycle used in FIG. 56A (obtained using an on-time (tₒₙ) and off-time (t_{off}) of the transducer 22 of 33 ms and 33 ms, respectively, with a 15 Hz PRF) was reduced to a duty cycle of 35% in FIG. 56C (by using a 4.1 ms on-time and a 7.66 ms off-time, with an 85 Hz PRF), keeping the drive amplitude of the transducer 22 the same. However, whilst the 50% duty cycle in FIG. 56A achieved a 12 mm maximum cleaning diameter, surprisingly, the 35% duty cycle of FIG. 56C achieved the same cleaning performance, with a 12 mm maximum cleaning diameter. Thus, reduction in cleaning did not scale with reduction in the duty cycle.

Additionally, changing the pulsing regime in the stated manner from the regime in FIG. 56A to those in FIGS. 56B and 56C caused a significant reduction in instabilities in the stream, as can be seen by comparison of the streams shown in FIGS. 56A-C. In FIG. 56A, three distinct pearls can be seen in the stream, with a severe narrowing of the stream around 2 cm from the nozzle 14, and a modest narrowing 1 cm from the nozzle 14, both of which hinder the transmission of acoustic energy down the stream, particularly when they make the cut-off frequency of the lowest mode exceed the operating frequency. The transducer pulsing regime shown in FIG. 56C, with an on-time of 4.1 ms, an off-time of 7.66 ms, and a 35% duty cycle, produced cleaning performance that was equivalent to that of FIG. 56A, with a much lower duty cycle and less incidence of instabilities on the wall of the stream 7. However, the instabilities resulting from the pulsing regime of FIG. 56C are not as suppressed as in the regime of FIG. 56B with an on-time of 2.0 ms, a 15 ms off-time, and a 12% duty cycle, and it is seen that the pulsing regime in FIG. 56C also created spitting from the nozzle 14. The pulsing shown in the upper panel of FIG. 56B did not produce significant spitting, and thus, the pulsing regime in FIG. 56B (with its greatly reduced on-time compared to FIG. 56A) was sufficient to allow more complete reduction of instability production at the nozzle 14. The results shown in FIGS. 56A-C demonstrate how strategic control of the on-time and off-time of the transducer pulsing can affect cleaning performance and spitting reduction. These results show that cleaning performance does not directly scale with transducer duty cycle, and surprisingly, equivalent or near-equivalent cleaning can be achieved at a much lower duty cycle. This permits significant reduction of the time-average power requirement (averaged over many seconds) and heat production of the transducer 22.

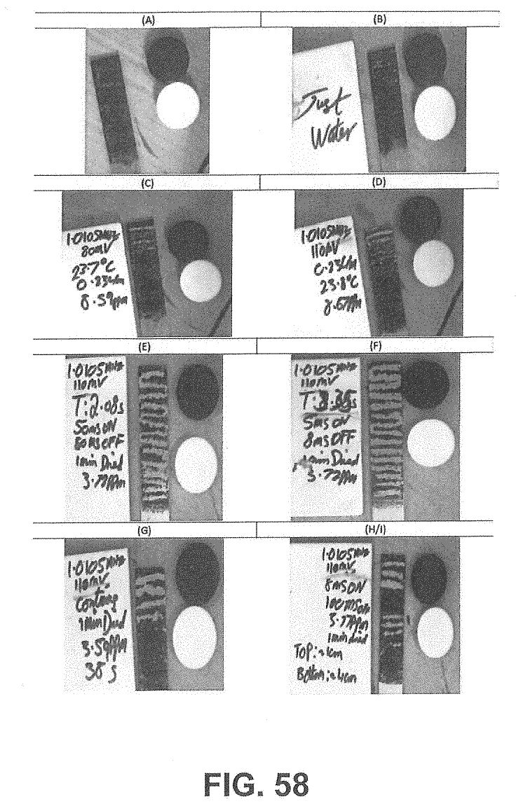

Similar effects will be seen at other carrier frequencies, but it is noted that scaling is not proportionate. One reason for this is that the frequency of the surface wave on the stream produced by the instabilities discussed herein is much less than the frequency of the ultrasound transmitted by the transducer, and so the use of pulsing regimes to suppress the instabilities is determined by the rise times of these instabilities, their wavelengths and periods, and the speed at which they propagate away from the nozzle 14. FIG. 58 illustrates performance in cleaning mascara from aluminum for a variety of different conditions and transducer operations, and Table 9 shows these testing results and parameters. For example, as shown in FIG. 58, use of a ˜1 MHz acoustic carrier wave frequency and a flow speed of 60 cm/s exiting a 2 mm diameter nozzle, in a pulsing regime with a 5 ms on time and an 8 ms off-time, can reduce instabilities because the on-time is short enough to enable the instabilities not to grow sufficiently. This enables good cleaning, as seen in Panel F of FIG. 58, in which 46% of the material was removed (See Table 9). It is noted that complete cleaning in this experiment would leave around 50% of the contaminant, because the standard cleaning path did not paint out contiguous stripes of cleaning, but rather tracked the stream across stripes of fixed separation with the expectation of leaving mascara between them to provide optical contrast. Similarly, a 50 ms on-time and a 50 ms off-time can cause the instabilities not to be present on the stream at the nozzle when the on-time starts, because the off-time is sufficiently long to have pearls propagate away from the nozzle in the manner of FIG. 56A before the next on-time begins. The cleaning performance for this pulsing regime is shown in panel (E) of FIG. 58 and reported in Table 9, with 55% of the contaminant remaining. Other pulsing combinations examined in the testing shown in FIG. 58 and Table 9 were not as effective at cleaning as the regimes in Panels E and F. It is contemplated that this is because the other pulsing regimes resulted in the stream attenuating the sound more between the nozzle 14 and the target. Using an 8 ms on-time with a 100 ms off-time, shown in Panel (H) of FIG. 58, does not generate the instabilities in the stream, but has such a low duty cycle that the proportion of time during a test that the ultrasound is on for, is too low to match the cleaning efficiencies of the regimes in Panels (E) and (F). Table 9 shows that the pulsing regime in Panel (H) resulted in 66% of the contaminant remaining after cleaning. Using a 50 ms on-time with an 8 ms off-time has too short an off-time to allow the stream surface waves that built up during the on-time to propagate away from the nozzle 14 (in the manner of FIG. 56A) before the onset of the next pulse, so that the cleaning and treatment of the target several cm away from the nozzle 14 is compromised by the stream surface waves.

Panels (C) and (D) of FIG. 58 show continuous-wave acoustic energy, which does not have any off-time, and thus, does not have the opportunity for a region of pearl-free stream to develop at the nozzle 14. Table 9 shows that such continuous-wave ultrasound allowed 71-75% of the contaminant to remain after cleaning, but that some pulsing regimes can enhance cleaning, despite reducing the acoustic energy delivered to the target over several seconds through the introduction of a finite off-time. This is consistent with the data observed from FIGS. 56A-C. The precise values of the optimal pulse on-times and off-times will depend on the acoustic frequency, the flow speed, the dissolved gas content and free bubble void fraction in the water supply, and the amplitude of the acoustic field at the nozzle 14, at the target, in the stream, and in the chamber 6 in front of the transducer 22.

Table 9 below shows the percentage of contaminant (mascara) remaining after different forms of cleaning treatment, versus the original condition. Water of various types were used, with the following abbreviations used in Table 9: RTTW=Room Temperature Tap Water; DOL=Dissolved Oxygen Level; and DW=Degassed Water. The water was passed through a nozzle of 2 mm inner diameter. The target is at 1 cm from the nozzle 14 unless otherwise stated, i.e., in Example (I).

TABLE 9

| FIG. 58 | Acoustic | | Input | | | Flow | | |
|---|---|---|---|---|---|---|---|---|
| Cleaning | Energy | Frequency | Voltage | DOL | Temp. | Rate | Water | % Mascara |
| Method | Condition | (MHz) | (mV) | (ppm) | (° C.) | (L/Min) | Type | Remaining |
| A | Before | — | — | — | — | — | — | 96 |
| B | Water | — | — | 8.22 | 23.1 | 0.23 | RTTW | 85 |
| | washed | | | | | | | |
| | without | | | | | | | |
| | ultrasound | | | | | | | |
| C | Continuous | 1.0105 | 80 | 8.39 | 23.7 | 0.23 | RTTW | 75 |
| | Ultrasound | | | | | | | |
| D | Continuous | 1.0105 | 110 | 8.67 | 23.8 | 0.23 | RTTW | 71 |
| | Ultrasound | | | | | | | |
| E | Pulsed | 1.0105 | 110 | 3.72 | 23.1 | 0.23 | DW | 55 |
| | Ultrasound | | | | | | | |
| | 50 ms On | | | | | | | |
| | 50 ms Off | | | | | | | |
| F | Pulsed | 1.0105 | 110 | 3.72 | 23.4 | 0.23 | DW | 46 |
| | Ultrasound | | | | | | | |
| | 5 ms On | | | | | | | |
| | 8 ms Off | | | | | | | |
| G | Continuous | 1.0105 | 110 | 3.59 | 22.9 | 0.23 | DW | 63 |
| | Ultrasound | | | | | | | |
| H | Pulsed | 1.0105 | 110 | 3.77 | 23.3 | 0.23 | DW | 66 |
| | Ultrasound | | | | | | | |
| | 8 ms On | | | | | | | |
| | 100 ms Off | | | | | | | |
| I | Pulsed | 1.0105 | 110 | 3.77 | 23.7 | 0.23 | DW | 71 |
| | Ultrasound | | | | | | | |
| | 8 ms On | | | | | | | |
| | 100 ms Off | | | | | | | |
| | (target 4 cm | | | | | | | |
| | from nozzle) | | | | | | | |

Table 10 below illustrates how various insonification conditions can affect the stream characteristics, including results for the distance to the first narrowing between pearls, and the distance to stream breakup, for a stream of 2 mm diameter at the nozzle 14. Various water types were used in these examples, with the following abbreviations being used in Table 10: RTTW=Room Temperature Tap Water; DOL=Dissolved Oxygen Level; and DW=Degassed Water. The data is shown for streams without acoustic energy, with continuous acoustic energy, and with various pulsing regimes with a variety of on-time and off-time combinations, all shown for the example ultrasonic frequency of 1.0105 MHz, and with various different acoustic energy amplitudes (for which the input voltage stands as approximate proxy).

TABLE 10

| | | | | Pulse | Pulse | Dissolved | Distance | Distance |
|---|---|---|---|---|---|---|---|---|
| | | Acoustic | Input | On- | Off- | Oxygen | prior to | prior to |
| | Water | Energy | Voltage | Time | Time | Level | narrowing | breakup |
| Test | Type | Condition | (V) | (ms) | (ms) | (ppm) | (cm) | (cm) |
| 1 | RTTW | None | — | — | — | 8.01 | 11 | 12 |
| 2 | RTTW | Continuous | 110 | — | — | 8.03 | 8 | 6 |
| 3 | DW | None | — | — | — | 3.55 | 11 | 12 |
| 4 | DW | Continuous | 110 | — | — | 3.61 | 1 | 1 |
| 5 | DW | Continuous | 20 | — | — | 3.64 | 10 | 11 |
| 6 | DW | Continuous | 30 | — | — | 3.69 | 8 | 10 |
| 7 | DW | Continuous | 40 | — | — | 3.74 | 8 | 9 |
| 8 | DW | Continuous | 50 | — | — | 3.77 | 7 | 9 |
| 9 | DW | Continuous | 60 | — | — | 3.79 | 6 | 8 |
| 10 | DW | Continuous | 70 | — | — | 3.81 | 6 | 8 |
| 11 | DW | Continuous | 80 | — | — | 3.83 | 5 | 8 |
| 12 | DW | Continuous | 90 | — | — | 3.84 | 4 | 7 |
| 13 | DW | Continuous | 100 | — | — | 3.88 | 3 | 6 |
| 14 | DW | Continuous | 110 | — | — | 3.92 | 2 | 5 |
| 16 | RTTW | Duty Cycle | 110 | 500 | 500 | 8.05 | 5 | 9 |
| 18 | RTTW | Duty Cycle | 110 | 50 | 50 | 8.10 | 7 | 10 |
| 20 | RTTW | Duty Cycle | 110 | 5 | 5 | 7.95 | 5 | 7 |
| 22 | RTTW | Duty Cycle | 110 | 5 | 8 | 7.99 | 8 | 10 |
| 24 | RTTW | Duty Cycle | 110 | 5 | 20 | 7.99 | 9 | 10 |
| 26 | RTTW | Duty Cycle | 110 | 5 | 40 | 7.94 | 10 | 11 |
| 28 | RTTW | Duty Cycle | 110 | 5 | 60 | 8.08 | 10 | 11 |
| 30 | RTTW | Duty Cycle | 110 | 5 | 80 | 8.05 | 10 | 11 |
| 32 | RTTW | Duty Cycle | 110 | 5 | 100 | 7.98 | 10 | 11 |
| 34 | RTTW | Duty Cycle | 110 | 1 | 30 | 8.06 | 8 | 10 |
| 36 | RTTW | Duty Cycle | 110 | 3 | 90 | 7.86 | 9 | 10 |
| 38 | RTTW | Duty Cycle | 110 | 8 | 100 | 7.89 | 10 | 11 |
| 40 | RTTW | Duty Cycle | 110 | 11 | 100 | 7.86 | 10 | 11 |
| 42 | RTTW | Duty Cycle | 110 | 50 | 200 | 8.11 | 6 | 8 |

Tables 9 and 10 illustrate the compromises between the various parameters that must be considered to optimize surface cleaning (or other desired surface treatment) to various target distances from the nozzle 14, e.g., acoustic energy condition or pulsing regime, acoustic energy amplitude and frequency, etc. While a reduction in the acoustic power to below the level that stimulates surface waves on the stream can cause the stream to propagate further without break-up, the acoustic power must be sufficiently high so that the amplitude of the acoustic energy that then reaches the target is sufficient to cause cleaning. In other words, acoustic energy with smaller amplitude and power permits greater target distances without stream breakup, but decreases cleaning efficiency at the target.

The apparatus 2 may also include the controller 19 being configured to control the transducer 22 to operate at the correct pulsing frequency and off-time, and to emit acoustic energy having the correct amplitude and frequency based on the properties of the body 4 and the liquid. The controller 19 may also be configured to receive input to manually adjust these and other parameters and/or may include algorithms for automatically calculating such parameters. Such pulsing of the acoustic energy does not need necessarily to turn the sound field off between pulses, but instead may modulate the acoustic energy, by amplitude or frequency modulation, to provide high energy acoustic pulses separated by low energy background. It is therefore understood that “on” as used herein refers to periods of sufficiently high energy acoustic generation to produce the effects described above, and “off” as used herein refers to periods of either no generation of acoustic energy or low energy acoustic generation.

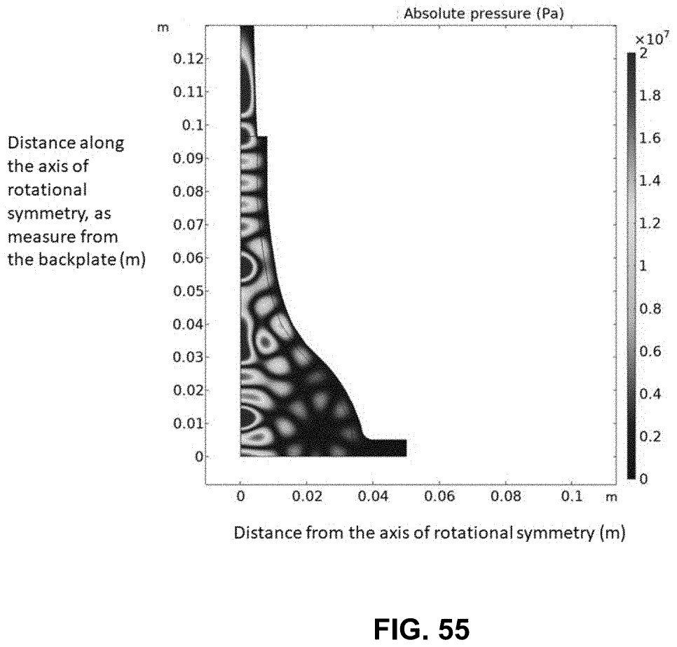

In one embodiment, the structure of the body 4 and the operation of the transducer 22 may be configured to produce the intentional establishment of modes and resonances in the chamber 6. This requires control of various dimensions and operating parameters and can greatly enhance or compromise the transmission of sound into the stream. FIG. 55 illustrates modes and resonances within the liquid in the chamber 6 and exiting in the stream, as well as within the body 4 itself, according to one embodiment. FIG. 55 illustrates the absolute acoustic pressure at 133.8 kHz for a half-space of the body 4 according to Profile 1 in FIG. 12. The simulation uses a body 4 made from a rubber material with an acoustic impedance equal to that of water, surrounded by air, such that the interface between the body 4 and the air is pressure-release, and the sound field therefore tends to zero. The rubber material in this configuration does not significantly absorb the acoustic energy at these frequencies over the two-way travel path through the rubber from the water/rubber interface to the air/rubber interface and back again. As seen in FIG. 55, the modal sound field in the chamber 6 and within the liquid will contain a number of acoustic pressure nodes and antinodes, to which bubbles will be attracted or from which they will be repelled through Bjerknes forces. Accordingly, while the transducer 22 is emitting acoustic energy, bubbles can be held (by the primary Bjerknes force) in positions that scatter the sound field. This is particularly an issue within the nozzle 14, where bubbles can be held at acoustic pressure antinodes (for bubbles smaller than resonance) and pressure nodes (for bubbles larger than resonance) when the sound field is on, acting as acoustic “shieldwalls” and preventing sound from reaching the stream. When the transducer 22 is switched off, the liquid flow will flush such bubbles out of the nozzle 14 and into the stream. Accordingly, the transducer 22 may be pulsed such that the off-time is long enough to remove such bubbles from the nozzle 14, as well as long enough for instabilities in the stream (if they are not removed through some other method) to propagate away from the nozzle 14 without being replaced at the nozzle 14, or to be damped out. This may be accomplished by using short on-times and long off-times for the transducer 22. Cleaning is a relatively slow process, compared to the time for a single ultrasonic carrier wave cycle, if pulsing did not change the efficiency of cleaning, then it would scale with the duty cycle. Any beneficial departure from that scaling is due to the ability of pulsing to enhance cleaning and other beneficial effects (see Table 9).

It is understood that the apparatus 2 of FIGS. 1-11 and other apparatuses described herein may additionally include one or more bubble generators, e.g., positioned within or proximate to the nozzle 14, such as in International Publication Nos. WO 2018/228848 and WO 2011/023746 that are incorporated herein by reference. The use of a bubble generator can assist in keeping the acoustic propagation path, as much as is feasible, clear of bubbles when the transducer 22 is on. This use also assists in produces a beneficial effect on the target by acoustically activating bubbles there, because if the bubbles are acoustically activated anywhere else in the apparatus 2 (except for the purpose of bubble generation), they attenuate the acoustic field and are detrimental to the acoustic energy reaching the bubbles when they are in close proximity to the target. Such a bubble generator (not shown) may be configured to produce bubbles predominantly of a size to be considered resonant bubbles and not unwanted bubbles, so that they generate the desired beneficial effects on the target. It is also important not to produce resonant sized bubbles in too high of numbers that may cause attenuation of the acoustic waves. Additionally, the bubble generator may be configured to produce bubbles in clouds or boluses that are timed so that as they are convected into the stream to produce regions of relatively bubble-free water. Further, the timing of the bubble generation and transducer 22 activation can be coordinated (taking into account their different propagation speeds in the stream) to ensure that the stream contains either bubbles, or acoustic waves, but not both at the same time, until the bubbles reach the target. In one embodiment, a fixed time interval between the bubble generation and the transducer 22 activation determines the range at the nozzle 14, for a given liquid flow speed out of the nozzle 14, at which the target can be placed for optimum cleaning (or other treatment). The apparatus 2 may include a device (not shown) to assist in correct positioning of the target such as crossed light beams or lasers that meet at the correct range, or optical sensors which activate the stream and/or transducer at the correct range, or an indicator light or sound when the target is at the correct position. In another embodiment, the crossing point of two or more converging liquid streams can also help constrain the target to the zone of optimal cleaning. In another embodiment, an acoustic pulse (of sufficiently low amplitude and high frequency to avoid inertial cavitation) could be emitted by the transducer 22, and the range to the target inferred from the two-way travel time for the acoustic pulse to echo back from the target, although this method can be difficult to implement when bubbles are in the liquid path. In a further embodiment, staggered time interval between the bubble generation and the ultrasonic activation (e.g. steadily increasing between pulses) enables the zone of optimal cleaning/treatment to sweep through a range of distances from the nozzle 14, to accommodate an apparatus or target that has less precise control on the range from the nozzle 14 to the target. The timing of bubble generation may be timed to generate bubbles that propagate similarly to the bubbles produced by acoustic energy within the body 4, or the operation of the transducer 22 can be controlled such that the acoustic energy does not produce bubbles, so that the apparatus relies on bubbles produced by the bubble generator alone.

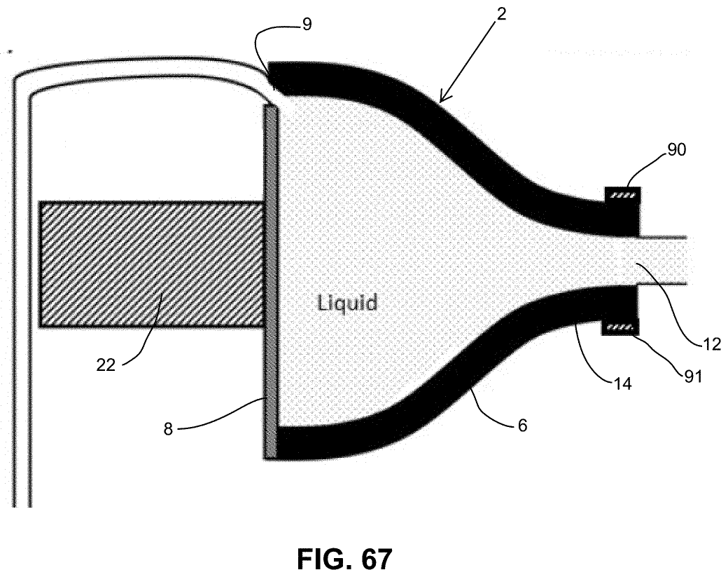

FIGS. 67-70 illustrate embodiments of apparatuses 2 that have bubble generators 90 within or proximate to the nozzle 14, which may also be proximate to the orifice 12. The closer the bubble generator 90 is to the point where the stream 7 exists the nozzle 14, the shorter the off-time for the transducer 22 required to allow those bubbles to travel in the stream 7 to the target in the absence of acoustic energy. As described herein, the transducer 22 activation can be timed so that the acoustic energy reaches the target at the same time as the bubbles, to activate the bubbles to clean or treat the target. The bubble generator 90 activation and deactivation can also be timed so that the bubble generator 90 is turned off when the transducer 22 is active. Operation of the bubble generator 90 in this manner helps ensure that no (or minimal) additional bubbles are in the acoustic propagation path between the transducer 22 and the target when the transducer 22 is active, avoiding disruption of the acoustic energy. As described herein, acoustic energy within the body 4 while the transducer 22 is active can also generate bubbles, and the operation of the bubble generator 90 and/or the transducer 22 may be controlled to account for these bubbles. In one embodiment, the operation of the bubble generator 90 may be timed to generate bubbles that propagate similarly to and/or concurrently with the bubbles produced by the acoustic energy within the body, such that the bubbles generated by the transducer 22 and the bubbles generated by the bubble generator 90 arrive at the target at approximately the same time. In another embodiment, the operation of the transducer 22 (e.g., tₒₙ, amplitude) can be controlled such that the acoustic energy is not sufficient to generate inertial cavitation to produce bubbles within the body 4 and does not stimulate instabilities and spitting in the stream, so that the apparatus 2 relies on bubbles produced by the bubble generator 90 alone.

Placement of the bubble generator 90 at or near the nozzle 14 provides multiple advantages. For example, the off-time in the pulsing of transducer 22 required to flush bubbles from the site of bubble generation and onto the target is reduced, because bubbles generated proximate the nozzle 14 do not need to travel through the length of the chamber 6. As another example, the use of the bubble generator 90 enables the transducer 22 to be operated in a manner where the transducer 22 does not generate bubbles. This, in turn, reduces the build-up of acoustically attenuating bubbles in the body 4 and the nozzle 14 by reducing the strength of Bjerknes forces that hold these attenuating bubbles in place, thereby increasing the likelihood that the liquid flow can flush the bubbles into the stream. Driving the transducer 22 at lower amplitudes and shorter on-times (tₒₙ) also reduces heat generation and power consumption of the transducer 22. It is understood that embodiments of bubble generators 90 other than those illustrated in FIGS. 67-70 may be used, that the apparatus 2 may include multiple bubble generators 90 of a single or multiple types, and that one or more bubble generators 90 may be located elsewhere on the apparatus 2, in other embodiments.

FIG. 67 illustrates one embodiment of an apparatus 2 that includes a bubble generator 90 in the form of a piezoelectric transducer 91 positioned at the nozzle 14 proximate to the orifice 12. The piezoelectric transducer 91 in FIG. 67 is shaped as a short length of tubing fitting over the tip of the nozzle 14 at the orifice 12, with opposite polarizations. In one embodiment, the tube-shaped piezoelectric transducer 91 is polarized on the curved inner and outer walls 91A with opposite polarities, but not polarized on the flat longitudinal ends 91B. In another embodiment, the piezoelectric transducer 91 is polarized with opposite polarities on the longitudinal ends 91B and not polarized on the curved walls 91A. The piezoelectric transducer 91 in these configurations can generate pulses through short bursts of inertial cavitation to produce a bolus of bubbles that travel down the stream 7 when the transducer 22 is not activated (during the off-time). The transducer 22 is then activated when those bubbles reach the target to be cleaned or treated, and after those bubbles have been rinsed away and cease treating the target, the bubble generator 90 is again activated for a short time to repeat the cycle.

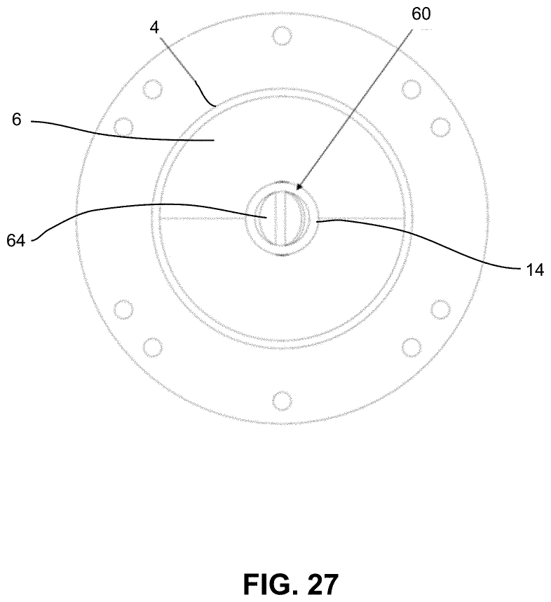

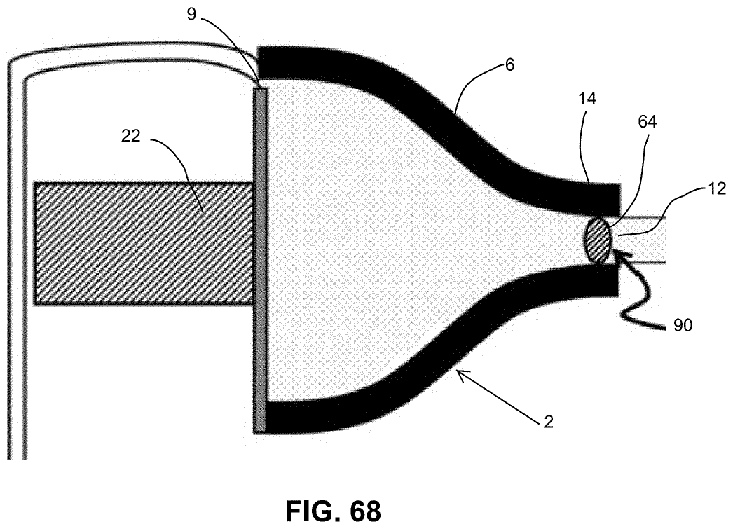

FIG. 68 illustrates another embodiment of an apparatus 2 that includes a bubble generator 90 embedded within the nozzle 14 proximate to the orifice 12, in the form of electrolysis electrodes or a piezoelectric transducer. In a configuration where the liquid has high conductivity (e.g., due to salt or other electrolyte content), wire electrodes can be used with no additional electrolyte. In a configuration where the water has a lower conductivity, a solid conductive membrane such as a polymer electrolyte membrane (PEM) can be used, with the electrodes on opposite sides of the membrane. By keeping the polymer membrane thin, the electrodes are close together, allowing a large potential gradient between them, which helps electrolyze the water outside of the membrane and in contact with the electrodes. The anode electrode electrolyzes the water and forms oxygen and positive charged hydrogen ions (protons). Then, the electrons flow in an external circuit and the hydrogen ions move across the PEM towards the cathode. Finally, hydrogen ions combine with electrons from the external circuit and form hydrogen bubbles at the cathode. One example of a suitable PEM material is a sulfonated tetrafluoroethylene-based fluoropolymer-copolymer (e.g., Nafion®). In one embodiment, the bubble generator 90 may be provided within the structure of a choke 60 in the nozzle 14, such as within the butterfly valve 64 shown in FIGS. 27-30.

FIG. 69 illustrates another embodiment of an apparatus 2 that includes a bubble generator 90 located at the nozzle 14 proximate to the orifice 12, in the form of microinjectors 92 that function to inject bubbles or liquid with entrained bubbles into the liquid within the nozzle 14. Such microinjectors 92 may be provided at multiple locations around the periphery of the nozzle 14 to ensure even distribution of bubbles. The microinjectors 92 should be kept free of debris to ensure proper function.

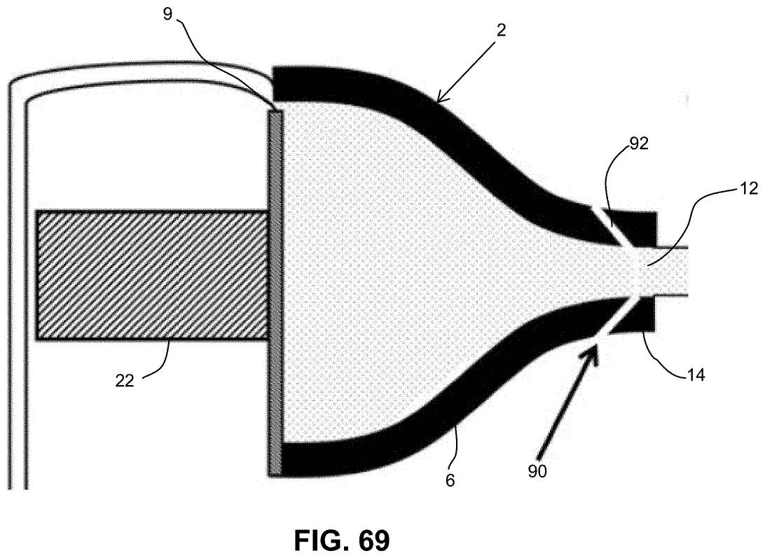

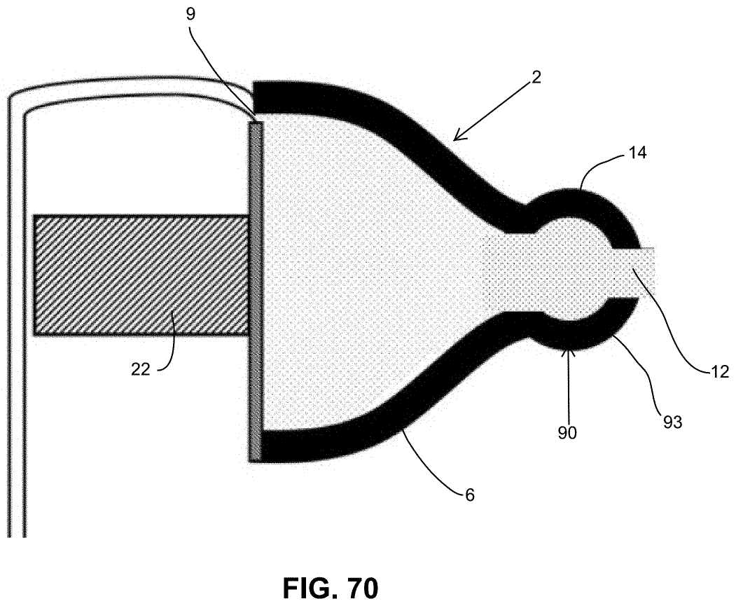

FIG. 70 illustrates another embodiment of an apparatus 2 that includes a strategically-shaped nozzle 14 that is configured to function as a bubble generator 90. In this embodiment, the nozzle 14 is shaped in a smooth, nearly-spherical bulge 93, which is designed to reflect the acoustic field produced by the transducer 22 to produce bubbles at the center of the bulge 93. The transducer 22 is operated to briefly increase amplitude or tuning frequency to cause the acoustic pressure amplitude at the center of the bulge 93 to exceed the threshold for inertial cavitation for a short period of time. This inertial cavitation generates bubbles, which are then swept out of the nozzle 14 and into the stream 7 by liquid flow. In another embodiment, the apparatus 2 may include an additional transducer (not shown) configured and positioned to create this inertial cavitation, such as a partially spherical transducer within the wall of the bulge 93.

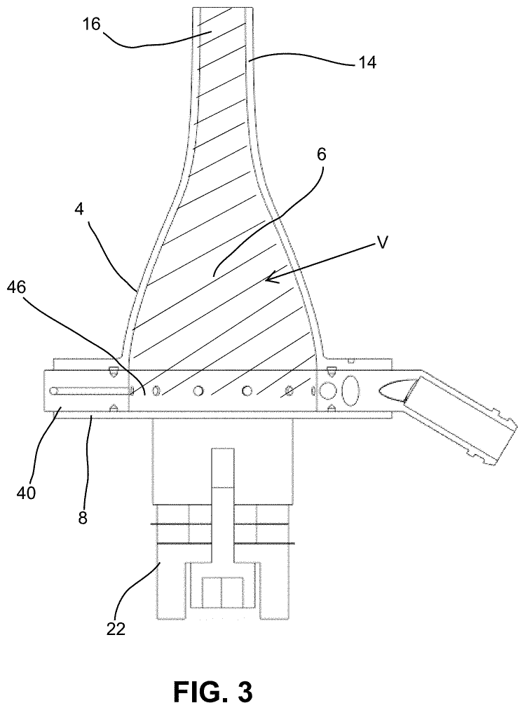

In the embodiment of FIGS. 1-11, at least two of the transducer 22 (or components thereof), the rear wall 8, and the body 4 may have the same cross-sectional shape when viewed along the length or axial direction. At least the forward-most surface of the transducer 22 (closest to the chamber 6, e.g., the head mass 51 in FIGS. 1-11) and/or the acoustic-emitting portion thereof, e.g., the piezoelectric element 50, have the same cross-sectional shape as the rear wall 8 and/or the body 4 in this embodiment. In the embodiment of FIGS. 1-11, the transducer 22, the rear wall 8, and the body 4 all have a circular cross-sectional shape when viewed along the length or axial direction. In this embodiment, the head and tail masses 51, 52 and the piezoelectric disc(s) 50 of the transducer 22 all have similar peripheral shapes (e.g., circular), although some or all of these components may have different diameters or peripheral widths. Additionally, the apparatus 2 may be configured such that the surface area A1 of the rear wall 8 that is exposed to the chamber 6 is dimensioned in an advantageous ratio to the surface area A2 of the front surface of the transducer 22 and/or the volume V of the chamber 6. These relative dimensions influence the energy and pulsing regime of the transducer 22 that is required to produce bubbles of the desired size and to energize the bubbles at the surface of the target, among other factors. In various embodiments, the ratio of the exposed area A1 of the rear wall 8 to the area A2 of the front surface of the transducer 22 is from 1.4:1 to 2.2:1, or from 1.5:1 to 2.1:1, or from 1.6:1 to 2.0:1, or from 1.7:1 to 1.9:1, and in another embodiment, the ratio of A1:A2 is about 1.8:1. Additionally, in various embodiments, the ratio of the volume V of the chamber 6 to the exposed area A1 of the rear wall 8 is from 38 mm³:1 mm² to 50 mm³:1 mm², or from 39.5 mm³:1 mm² to 48.5 mm³:1 mm², or from 41 mm³:1 mm² to 47 mm³:1 mm², or from 42.5 mm³:1 mm² to 45.5 mm³:1 mm², and in another embodiment, the ratio of V:A1 is about 44 mm³:1 mm². The calculated volume V is the sum of the internal volume of the cone and volume of the inner section of the manifold, it is described in FIG. 3. It is understood that these ratios are expressed with respect to identical units in the case of area/area ratios or units of the same magnitude in the case of area/volume ratios (e.g., mm³/mm²).

In another embodiment, the nozzle 14 may be configured with a narrow orifice 12 to provide a lower diameter stream, for example a 2 mm nozzle diameter. In this configuration, the ratio of the exposed area A1 of the rear wall 8 to the area A2 of the front surface of the transducer 22 in various embodiments is from 1:1 to 2.6:1, or from 1.2:1 to 2.4:1, or from 1.4:1 to 2.2:1, or from 1.5:1 to 2.1:1, or from 1.6:1 to 2.0:1, or from 1.7:1 to 1.9:1, and in another embodiment, the ratio of A1:A2 is about 1.8:1. Additionally, in one embodiment, the ratio of the volume V of the chamber 6 to the exposed area A1 of the rear wall 8 in this configuration is from 36 mm³:1 mm² to 54 mm³:1 mm², or from 37 mm³:1 mm² to 52 mm³:1 mm², or from 38 mm³:1 mm² to 50 mm³:1 mm², or from 39.5 mm³:1 mm² to 48.5 mm³:1 mm², or from 41 mm³:1 mm² to 47 mm³:1 mm², or from 42.5 mm³:1 mm² to 45.5 mm³:1 mm², and in another embodiment, the ratio of V:A1 is about 44 mm³:1 mm².

In a further embodiment, the nozzle 14 may be configured in a goose-neck configuration as described herein. In this configuration, the ratio of the volume V of the chamber 6 to the exposed area A1 of the rear wall 8 in various embodiments is from 48 mm³:1 mm² to 74 mm³:1 mm², or from 50 mm³:1 mm² to 70 mm³:1 mm², or from 54 mm³:1 mm² to 66 mm³:1 mm², or from 56 mm³:1 mm² to 64 mm³:1 mm², or from 58 mm³:1 mm² to 62 mm³:1 mm², and in another embodiment, the ratio of V:A1 is about 60 mm³: 1 mm².

In some applications, a flow rate of 2 L/min for a 10 mm diameter stream 7 may be used, although in other applications, a lower flow rate may be desired for various reasons. Flows as low as 1 L/min have been successfully used with a 1 cm diameter nozzle, and by decreasing the nozzle diameter to 2 mm, flows as low as 0.25 L/min have successfully been used. For example, higher flow rates can present water conservation issues and/or may risk damage to the target, in some applications. It is noted that in some regions higher flow rates (e.g. up to 6.5 L/min per nozzle) are acceptable and these have been successfully used, and with larger nozzle diameters, higher flow rates (e.g., 12 L/min per nozzle) may be used if allowed. Reducing the flow speed when the liquid exits the nozzle 14 can be used to reduce the volume flow rate, but reducing flow speed presents potential problems. For example, reducing the flow speed shortens the distance from the nozzle 14 at which the stream 7 narrows to a point where there is only evanescent pressure field beyond it preventing ultrasound of sufficient amplitude from reaching the target to generate surface waves on the “resonant” bubbles there. As another example, in some regimes, reducing the flow speed enhances the unwanted surface waves on the stream 7 that intermittently narrow the stream (i.e., creating pearling as described herein). Both these phenomena enhance the likelihood of stream breakup, preventing passage of the acoustic waves. To address this issue, the volume flow rate can be reduced while maintaining the flow speed with which the liquid exits the nozzle 14 by reducing stream diameter, i.e., reducing the size of the nozzle 14. Since the stream 7 thins under gravitation acceleration most rapidly with distance from the nozzle 14 when projected in a downwards orientation, a change of orientation may also be beneficial in increasing the range to which the acoustic energy can be projected. A reduction in stream diameter requires an increase in the frequency of the acoustic energy that can pass down the stream 7, as the cut-off frequency increases, because the stream 7 is a tapering waveguide with pressure-release walls. An increase in frequency of the acoustic waves, in turn, means the bubbles which are considered “resonant” bubbles will have smaller radii, as shown in FIG. 57A. It is therefore possible to reduce the flow rate from 2 L/min in the 1 cm diameter stream, using 135 kHz frequency to activate ˜40-60 μm diameter bubbles, by using narrower streams 7, with higher cut-off frequencies, necessitating the use of higher frequencies, which require smaller bubbles on which they can stimulate surface waves. Two example regimes are: using a 4 mm diameter stream with a flow rate of 0.5 to 2 L/min with bubbles of 8-15 μm diameter and a 700 kHz frequency; and using a 2 mm diameter stream with a flow rate of 0.25 to 1 L/min with bubbles of 4-12 μm and a 1 MHz frequency. It is understood that these are specific examples, and a continuum exists based on first determining the cut-off frequency f_{c} for the stream based on the stream radius (i.e., based on the internal diameter of the nozzle 14) necessary to achieve the desired flow rate. The cut-off frequency f_{c} for a liquid stream that flows through a gas space on leaving the nozzle, and which therefore has a boundary condition at the curved walls of the stream that is pressure-release (if the stream were an ideal cylinder of radius rₛ) is:

$${f}_{c} = \frac{2.4048 c}{2 {π r}_{s}}$$

where c is the sound speed in the liquid. Once the frequency is determined, the exact resonant bubble size (and from the damping, the tolerant range of bubble sizes around this on which surface waves may form) can be determined for the frequency, using known equations.

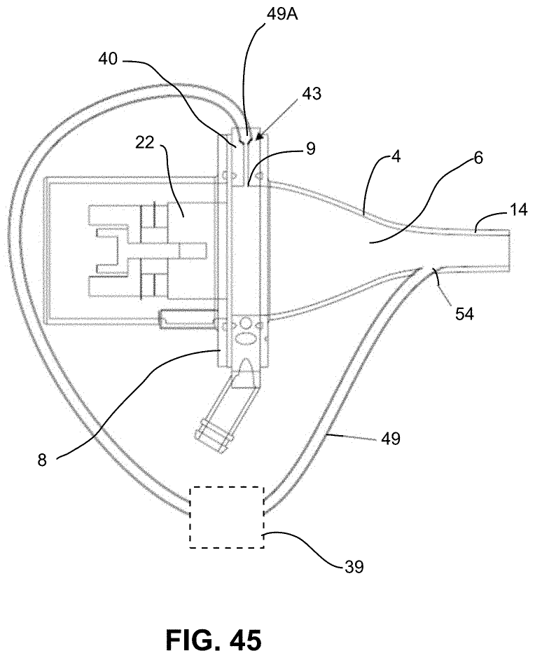

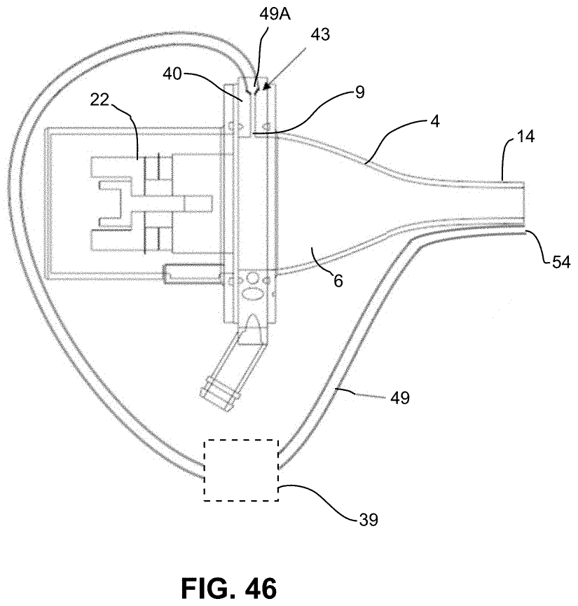

In one embodiment, the apparatus 2 has a venting system that includes one or more vents 9 in communication with the chamber 6 for removal of gas, as discussed herein. If air or other gas is allowed to build up within the chamber 6, the gas can block acoustic waves from being able to travel down the stream, diminishing or preventing the activation of bubbles down the stream. The vent(s) 9 may be positioned to be located at or near the uppermost point of the liquid in the chamber 6 during use, when such use has the body 4 in an orientation where the nozzle 14 points downwards or to the side or some angle in between, such as in the embodiment of FIGS. 1-11. Gas bubbles within the liquid in the chamber 6 rise to the uppermost point of the chamber 6, where they are removed by the vent 9. It is noted that in the embodiment of FIGS. 1-11, the apparatus 2 is designed to be used with the nozzle 14 pointed downward at an angle, making the location of the vent 9 the approximate uppermost point of the chamber 6 when the nozzle 14 points sideways and/or downward (including angles in between), such as shown in FIG. 1. The same is true of the embodiments in FIGS. 20-21 and 45-46. The venting system may also include an exhaust outlet 49A, such as the exhaust outlet 49A connected to the gas conduit 43 at the bottom of the manifold body 45 in the embodiment of FIGS. 1-11. The exhaust 49 may be directly connected to the vent 9 in another embodiment, such as in FIGS. 20-21. In one embodiment, the exhaust 49 is in the form of tubing and has an outlet 54 that is positioned below the level of the vent 9 during use and is sufficiently narrow to prevent air from entering the outlet 54, to enable creation of a siphon effect to continuously remove gas from the liquid in the chamber 6 during use. The outlet 54 may be positioned in a drainage location in one embodiment, such as in a drainage tub or a sink. Once a siphon is created at the vent 9, a mixture of liquid and gas will be continuously removed from the chamber 6. If large or numerous gas bubbles are visible in the liquid of the exhaust 49, the operator may wish to delay usage until the bubble incidence is reduced. The apparatus 2 may include a detector (not shown) that is light-based, conductivity-based, or acoustic-based (e.g., ultrasound) in one embodiment for sensing the presence of bubbles in the liquid removed by the siphon in the exhaust 49. The exhaust 49 in one embodiment may transport the liquid/gas mixture away from the apparatus, such as to a storage or disposal location, or to an apparatus for recycling the liquid back to the liquid supply conduit 20, which may include a degasser and/or an outgasser. In another embodiment, the exhaust 49 may transport the liquid/gas mixture to the stream 7 to create a Venturi effect. The configurations shown in FIGS. 45-46 and described herein are configured to be outfitted in this manner. In one embodiment an internal pump 39 (shown schematically in broken lines in FIGS. 45-46) could be added to assist in the removal of the bubbly liquid through the exhaust, to pull liquid from the body 4 into the vent 9.

In order to assist creation of the siphon in the exhaust 49, and in the initial filling of the chamber 6 and nozzle 14 with liquid at the onset of use, the apparatus 2 may include a choke 60 in one embodiment. The chamber 6 is initially filled with gas before the liquid flow is initiated from the inlets 18 into the chamber 6, and injection of the liquid into the chamber 6 forces gas out through the vent 9 and/or the nozzle 14. However, once the flow rate of the liquid entering the chamber 6 is equal to that exiting through the nozzle 14, the pressure within the cone is not sufficient to vent all the gas. The choke 60 can be used to restrict flow of liquid out of the nozzle 14 and thereby build pressure within the chamber 6 to force the remaining gas out through the vent 9. The action of the choke 60 also causes the initial flow of liquid through the vent 9 and into the exhaust 49 to begin the siphon. The operator can determine whether and when to use the choke 60 to restrict flow through the nozzle 14 based on the presence of liquid in the exhaust 49. If no liquid is detected in the exhaust 49 or if bubbles are detectable in the liquid flowing through the exhaust 49, then significant gas still exists in the chamber 6, and the choke 60 should be activated. Once the exhaust 49 is filled with liquid (without significant bubbles), the choke 60 can be released. The use of transparent tubing in the exhaust 49 may assist with this determination. If there is no liquid flow observed to come from the exhaust 49 at any point during operation, this is also an indication that choking is required.

The choke 60 can be triggered manually in one embodiment, such as through user judgment as described above. In another embodiment, the choke 60 can be activated at fixed times, such as on initial use of the device and automatically or manually at set time intervals (e.g., every 5 minutes) subsequently. This interval may increase or decrease when the water supply into the body 4 has fewer bubbles, e.g., because of local supply conditions). The time interval may be set manually or automatically based on the characteristics of the water supply. The apparatus 2 may further include a sensor (not shown), such as a light, acoustic (e.g., ultrasonic) or conductivity-based sensor, on the exhaust 49 and/or the liquid supply conduit 20. In embodiments where choking is done manually, an indicator (e.g., warning light) may be used to alert the operator to the need to activate the choke 60. Such an indicator may be activated based on a fixed time interval as discussed above, or from input from the sensor indicating presence of bubbles in the exhaust 49 or the water supply. In a further embodiment, choking can be done automatically by an automated actuator, which may be activated based on the triggering of the sensor. Such a sensor can alert an electronic controller 19 or transmit data that is received and analyzed by the controller 19, and the controller 19 can activate the actuator as necessary. The indicator described above may be activated in a similar manner.

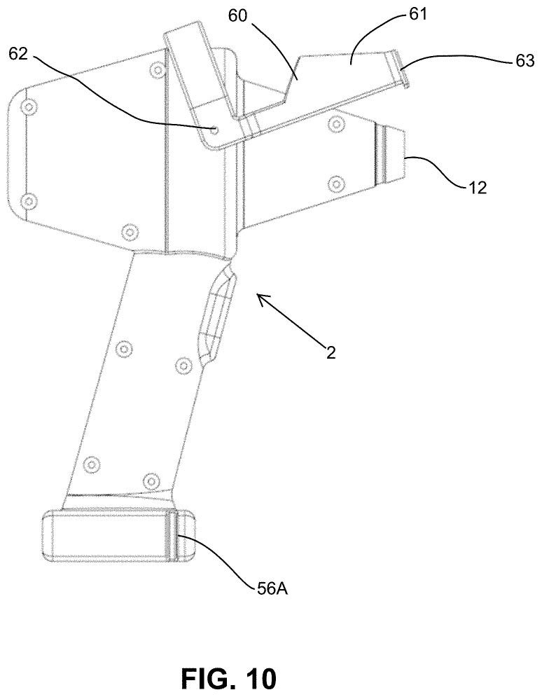

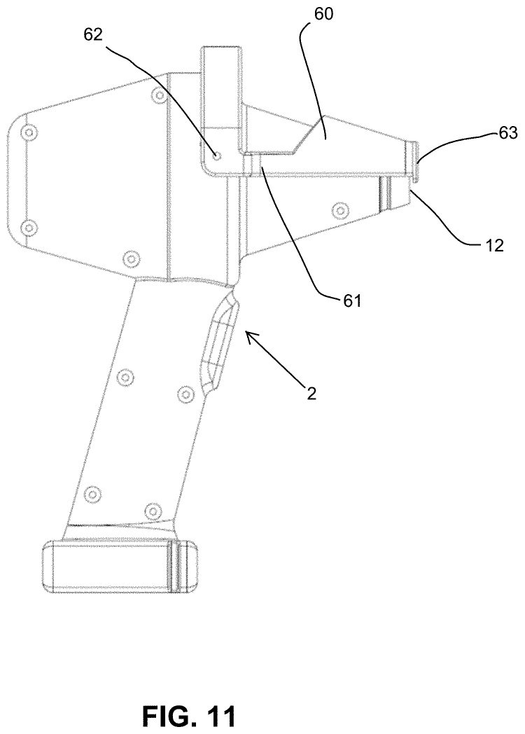

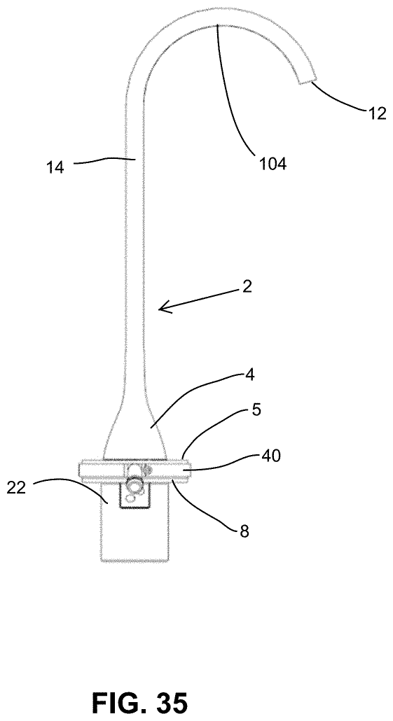

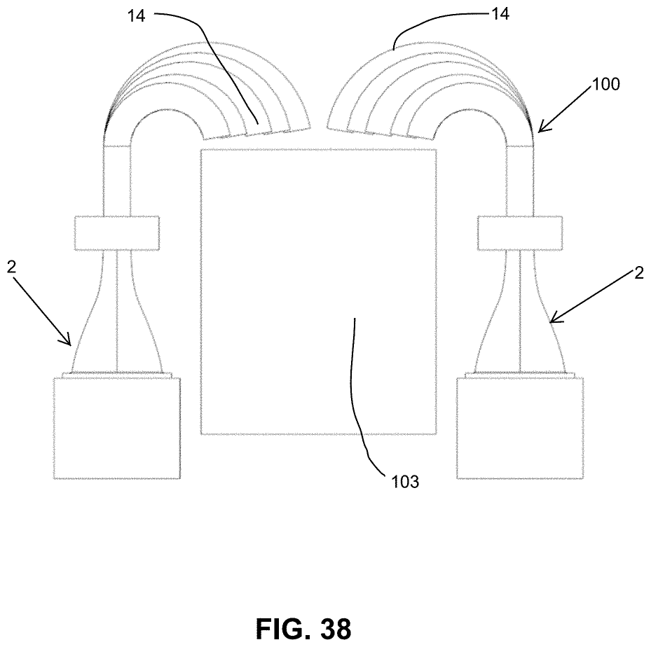

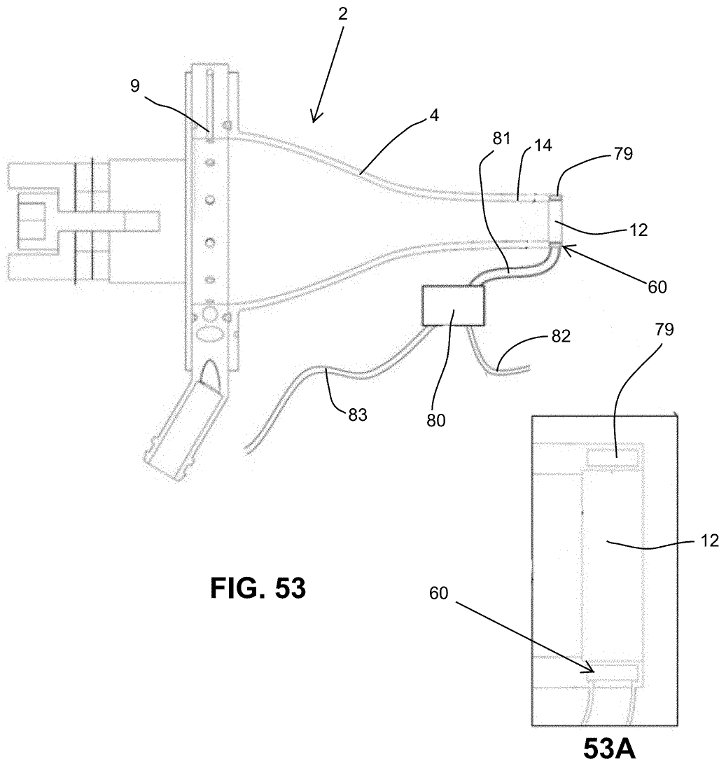

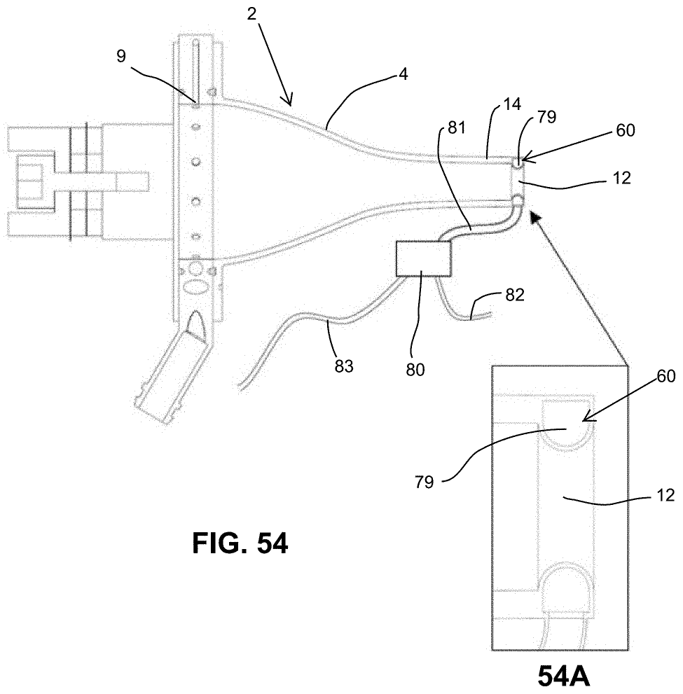

The apparatus 2 of FIGS. 1-11 includes an external choke 60 that pivots between a free position (see FIG. 10) where the choke 60 does not obstruct the flow through the nozzle 14 and a choking position (see FIG. 11) where the choke 60 restricts flow through the nozzle 14 to force gas and liquid out through the vent 9. The choke 60 in this embodiment includes a lever arm 61 connected to the casing 55 by at a pivot connection 62, with a restrictor 63 connected to the lever arm 61 and moveable with the lever arm 61. The lever arm 61 is manually moveable, and the restrictor 63 restricts flow through the nozzle 14 when the choke 60 is pivoted to the choking position. The restrictor 63 obstructs only a portion of the nozzle 14 in the embodiment of FIGS. 1-11, and the restrictor 63 may be moved to obstruct more or less of the nozzle 14 by pivoting. The apparatus 2 may include other types of choke mechanisms in other embodiments, including simple plugs or caps or various mechanical and electro-mechanical valve devices. In another embodiment, an inflatable bladder or a diaphragm (see FIGS. 53-54) may be used to restrict flow through the nozzle 14, which could be automatically or manually triggered by a solenoid. It is noted that in a usage where the liquid is forced upwardly out of the chamber 6, such as in FIGS. 35-40 described elsewhere herein, neither a vent 9 nor a choke 60 may be necessary, as the gas will exit through the nozzle 14.

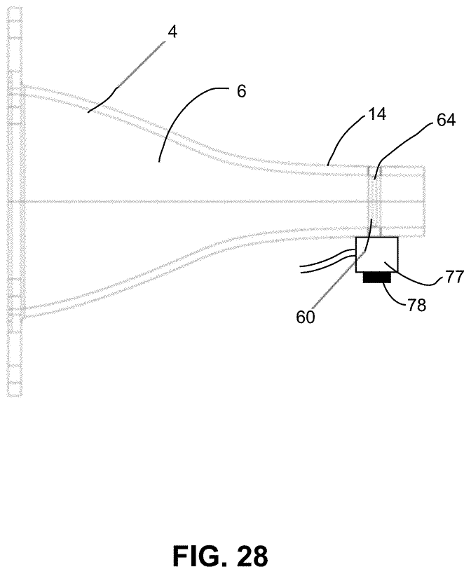

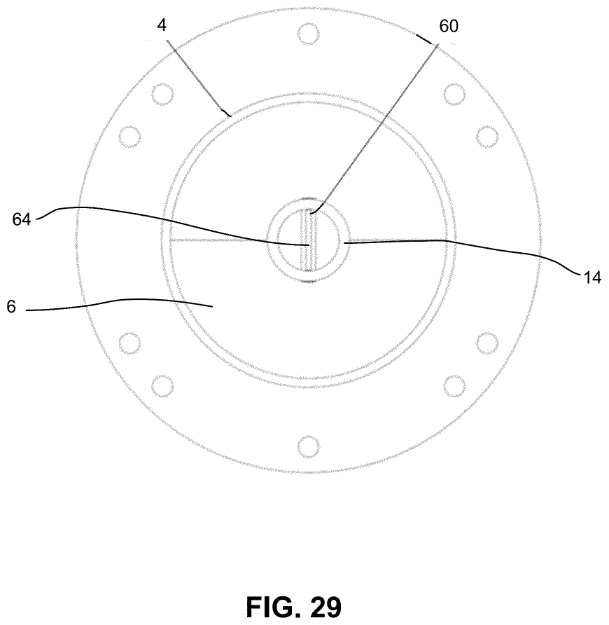

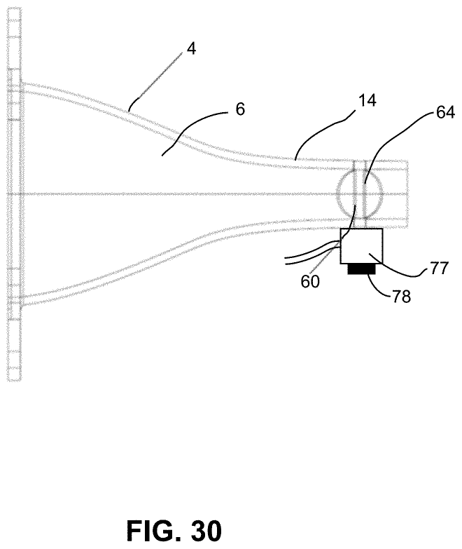

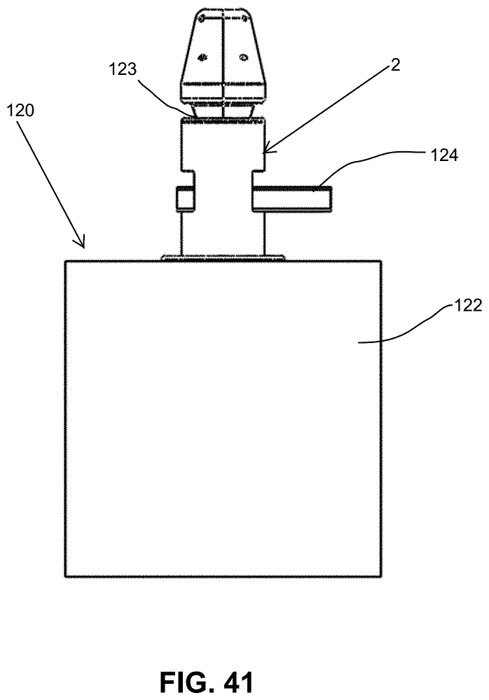

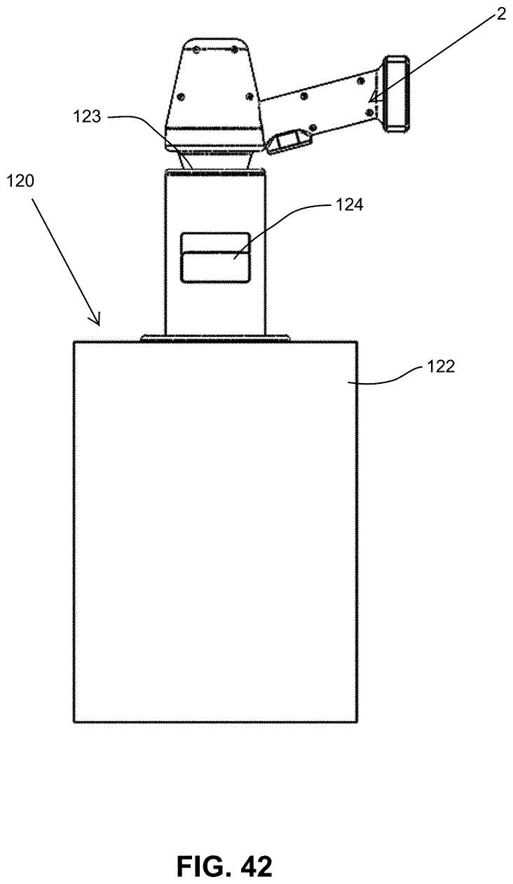

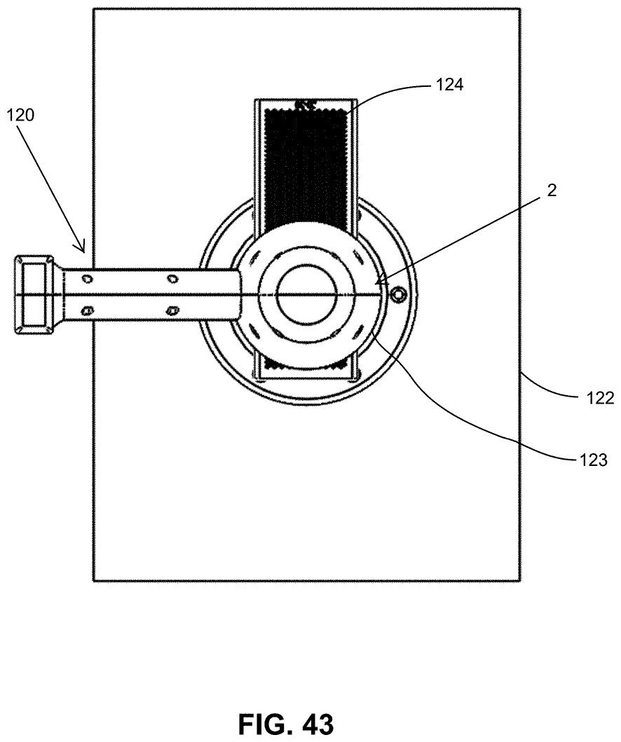

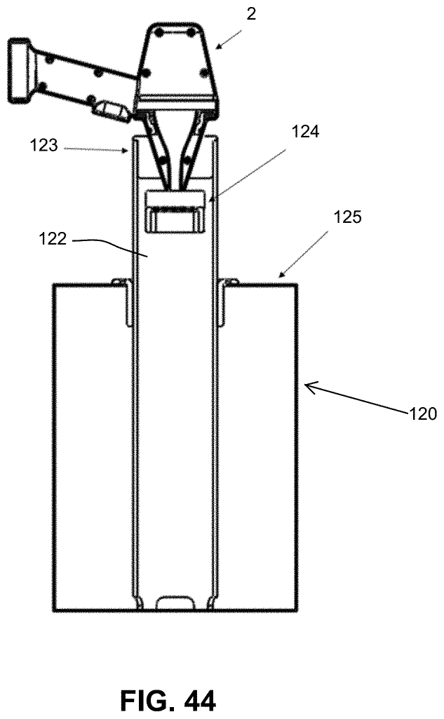

FIGS. 27-30 illustrate another embodiment of a conical body 4 usable with the apparatus 2 of FIGS. 1-11 that includes a choke 60 in the form of a butterfly valve 64 positioned within or upstream from the nozzle 14 to restrict flow through the nozzle 14. The butterfly valve 64 may be moveable by swiveling between a free position (see FIGS. 29-30) where the choke 60 does not obstruct the flow through the nozzle 14 and a choking position (see FIGS. 27-28) where the choke 60 restricts flow through the nozzle 14 to force gas and liquid out through the vent 9. The apparatus 2 may further include an activation mechanism for the butterfly valve 64, such as a mechanical/manual activation mechanism (e.g., a lever) or an electronic/automatic activation mechanism (e.g., a button). In one embodiment, shown in FIGS. 28 and 30, an electronic activation mechanism may include an electric motor 77 configured to activate the butterfly valve 64, which is connected to an encoder 78 to control the motor 77 to position the butterfly valve 64. A butterfly choke 60 can be particularly useful in situations where the nozzle 14 is inaccessible, such as due to the positioning or the usage of the apparatus. For example, the apparatus 2 may be positioned in a location that is difficult to physically access, or may be used in a biologically or chemically hazardous usage, such as by positioning the apparatus 2 above a testing chamber, e.g., as in FIGS. 41-44. The structure of a butterfly choke 60 can also be used to include extra functionality. In some circumstances, the valve 64 can act as a turbulence-tamer (along with potentially other turbulence-tamers that can be added elsewhere in the nozzle 14 and/or chamber 6) to create a more controllable stream. Such turbulence-tamers operate to remove turbulence by passing the flow through parallel channels, and sponges, porous stones, or metal meshes (not shown) are placed along the path to hinder the propagation of bubbles towards the outflow. In addition, a bubble generator (injector, pumped liquid containing previously-made bubbles, electrolysis, ultrasonic transducer, etc.) can be incorporated into the butterfly valve 64.

FIGS. 53-54 illustrate another embodiment of a conical body 4 usable with the apparatus 2 of FIGS. 1-11 that includes a choke 60 in the form of an inflatable member, such as a bladder 79 as shown in FIGS. 53-54 or a diaphragm, positioned within the nozzle 14 (e.g., around the orifice 12) to restrict flow through the nozzle 14. The bladder 79 may be inflatable and deflatable between a free position (see FIG. 53), where the bladder 79 is not inflated and the choke 60 does not obstruct the flow through the nozzle 14, and a choking position (see FIG. 54) where the bladder 79 is inflated such that the choke 60 restricts flow through the nozzle 14 to force gas and liquid out through the vent 9. The apparatus 2 may further include an activation mechanism 80 for the bladder 79, such as a mechanical activation mechanism or an electronic activation mechanism. The activation mechanism 80 in FIGS. 53-54 includes a solenoid that is connected to a pressure source 83 that selectively delivers a fluid (e.g., gas or liquid) through a conduit 81 to the bladder 79 for inflation, such as upon activation by the activation mechanism. An exhaust line 82 may also be connected to the activation mechanism 80, such that the activation mechanism 80 is configured to selectively place the pressure source 83 or the exhaust line 82 in communication with the bladder 79 to inflate or deflate the bladder 79, respectively. The bladder 79 may be provided as a generally compliant member or a member that is compliant only locally to the choking area.

In another embodiment, a non-obstructive choking technique may be used to clear gas from the chamber 6 and initiate the siphon effect. For example, the liquid may be introduced through the inlets 18 at a very high flow rate that is faster than the liquid can flow through the nozzle 14, creating a pressure differential in the exhaust 49 that is sufficient to accomplish this. In this embodiment, a short burst of liquid at a much greater volume flow rate into the chamber 6 can appropriately fill the chamber 6 and begin the operation of the exhaust 49 siphon, by increasing the initial liquid pressure in the chamber 6 above normal operating pressure. This can be accomplished by temporarily increasing the volume flow rate into the chamber 6 to a volume flow rate that is greater than the liquid can exit the nozzle 14 (or another similar method), which causes the chamber 6 to fill and flush out bubbles. By increasing the pressure inside the chamber 6, the liquid can fill the chamber with water faster than it is drained from the nozzle 14, without the need to obstruct the nozzle 14 with a choke. Using such methods, the increased pressure that builds up in the chamber 6 exhausts the air present within the chamber 6. This can be done either when initially filling the chamber 6, or when removal of excess gas is required in order to resume operation. If the pressure differential between the liquid within the body 4 and the atmosphere outside of the apparatus 2 is sufficient, and an exhaust 49 is used, the liquid will flow through the exhaust 49 and initiate the siphoning of air that enters the chamber 6. The temporarily increased liquid volume flow rate into the chamber 6 can be achieved, for example, by intermittently operating a solenoid value to open to allow a higher flow rate than normal for a short time. As another example, a Venturi system can use the flow of the fluid through the nozzle 14 to draw air from within the chamber 6. FIGS. 45-46 illustrate embodiments that utilize this effect. In FIG. 45, the exhaust 49 has an outlet 54 within the chamber 6 close to the nozzle 14, and in FIG. 46, the exhaust 49 has an outlet 54 outside the chamber 6 and adjacent to the nozzle 14. In either of these embodiments, a Venturi device (not shown) can be included in the liquid flow of the stream 7 to create a Venturi effect to draw gas and liquid through the vent 9 and the exhaust 49. This Venturi system could be used in connection with an increased liquid flow rate to enhance effectiveness. In a further embodiment, a pump 39 (shown schematically in broken lines in FIGS. 45-46) can optionally be connected to the exhaust 49 to actively pull gas and liquid through the vent 9 and the exhaust 49. The gas and liquid can be taken away from the apparatus 2 or reintroduced to the chamber 6 in this embodiment. In such embodiments, the exhaust 49 may be considered to be part of an exhaust system including the vent 9, the pump 39, and any conduits forming the exhaust 49 and extending to the outlet 54.

The apparatus 2 of FIGS. 1-11 is configured as a self-contained handheld delivery device and includes a casing 55 configured to facilitate handheld use. The casing 55 in this embodiment is shown in FIGS. 1, 10, and 11, and includes a handle 56 and a main housing 57 with an aperture 58 at one end. The casing 55 may be formed in various manners, including by use of two half-pieces that may be separate or configured as a clamshell piece, which are joined together by fasteners. The handle 56 is connected to the main housing 57 and is configured for gripping by the operator to move and direct the apparatus 2. The apparatus 2 in this embodiment also includes two buttons 59 located on the handle 56, which are used to initiate the liquid flow and the transducer 22, respectively. The main housing 57 encloses the transducer 22, the body 4, the rear wall 8, and the manifold 40, among other components, and these components are mounted fixedly within the main housing 57. The body 4 is positioned such that the nozzle 14 is located at the aperture 58 of the main housing 57, and the stream 7 passes through the aperture 58. The manifold inlet 42 extends from the main housing 57 into the handle 56, and the liquid supply conduit 20 extends upward through the handle 56 to connect to the manifold inlet 42. The exhaust 49 also passes downwardly from the main housing 57 through the handle 56 to exit the apparatus 2. In one embodiment, a shield (not shown) can be placed between the fingers and the target, to protect the fingers from splashing by potentially contaminated liquid. Optionally, the shield can be made detachable, such as in the form of a clip-on unit that passes over the nozzle 14 to form a shield in front of the user's fingers. A detachable configuration allows the shield to be removed and washed, or replaced if heavy contamination requires a disposable shield.

The apparatus 2 may include a controller 19, which is shown schematically in FIGS. 1 and 20. The apparatus 2 may require electronics to perform a number of tasks, including but not limited to: providing the signal that will drive the transducer 22, and any additional signal required for bubble generation if a separate bubble generator is used; electrical power amplification of those signals; control of the liquid supply, which in one embodiment (e.g., FIGS. 1-11) can be turned off and on using an electronic solenoid value; timing and delay circuits associated with operations (e.g., to not allow the ultrasound to be turned on when the chamber 6 is not full of liquid, such as by introducing a delay between turning on the water from the solenoid valve and disabling the switch to activate the ultrasound until the requisite number of seconds have passed, or an optical, acoustic (e.g., ultrasonic), or conductivity sensor on the exhaust 49 shows that it contains no detectable bubble interrupting a light beam, for example); safety and warning circuits associated with operations (lights to indicate transducer 22 warming, leading to disabling of the electrical power if heating is beyond tolerable bounds, e.g., 75° C.); data logging (hours used, water temperature, etc.) and other electrical operations. These can communicate to the switches, transducer and indicator lights in the casing via an umbilical, which may run alongside or with the liquid supply conduit 20. Some or all of these electronic devices can be incorporated within the casing 55 if space and electrical safety considerations allow, and/or at least some of these electronic devices may be located outside the casing 55, in various embodiments. These electronic devices may be collectively considered to constitute the controller 19, and may include at least a memory, a processor, and one or more electronic interfaces for transmitting and/or receiving signals from other devices and components. In one embodiment, the controller 19 may be configured to perform at least one or all of the following functions: generate drive signals to the power amplifier for driving the transducer 22; turn the flow of liquid on and off, and potentially control the flow rate; monitor the liquid flow; monitor the temperature at one or more locations; respond to button presses to control the liquid flow and the transducer 22; and report operation status by providing input to the operator. The liquid supply may be controlled by use of a solenoid valve controlled by an H-bridge in one embodiment. The temperature may be monitored by use of thermistors. Flow rate may be measured using a flow rate sensor, such as a sensor that produces electrical pulses at a frequency proportional to the flow rate. Input to the operator may be provided by use of multiple LEDs visible to the operator. It is understood that the controller 19 may include additional functionality as well, including communication with one or more devices external to the apparatus 2.

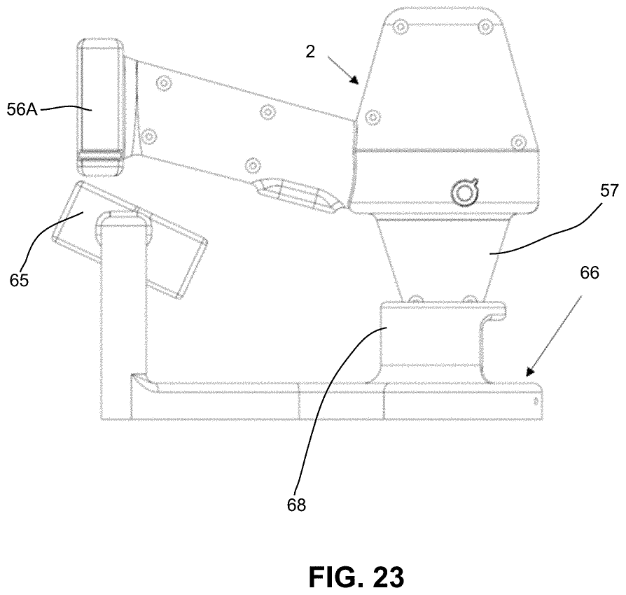

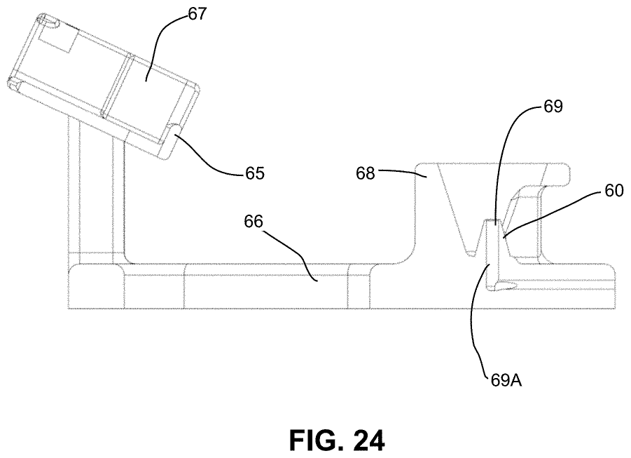

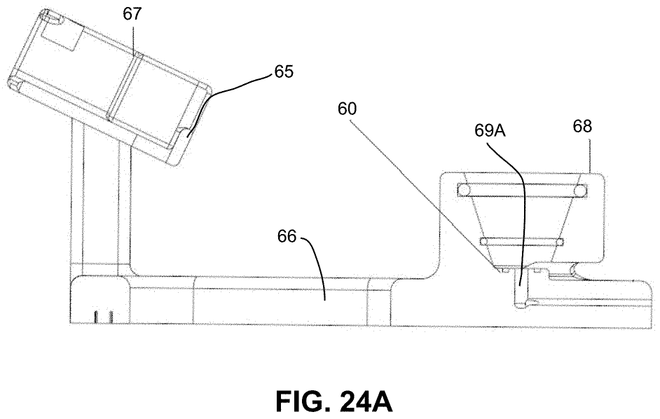

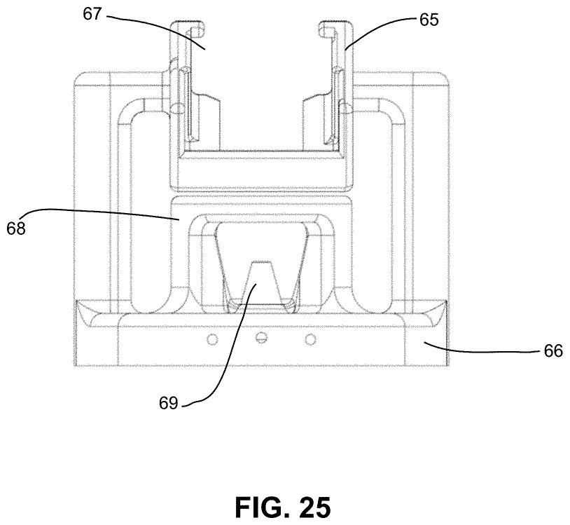

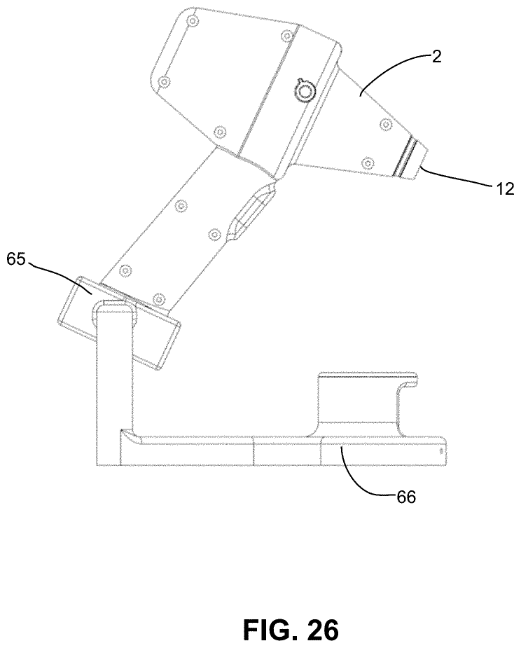

The apparatus 2 in FIGS. 1-11 may alternately be mounted on a holder 65 to assist in directing the stream 7 without risking dropping the apparatus 2. In one embodiment, the holder 65 may be mounted on a moveable and flexible arm (not shown). FIGS. 23-26 illustrate one embodiment of a holder 65 that is integrated into a stand 66 for holding the apparatus 2. The stand 66 is configured to support the holder 65 in a position to direct the stream downward at an angle as shown in FIG. 26. The holder 65 and the casing 55 may be dimensioned and structured in a mating or complementary manner to allow the holder 65 to engage the casing 55 and support the apparatus 2. In the embodiment of FIGS. 23-26, the casing 55 includes an enlarged portion 56A at the base of the handle 56, which is received and engaged within in a slot 67 in the holder 65 to fix the casing 55 in position. The holder 65 may have a pivoting capability in one embodiment. The stand 66 in FIGS. 23-26 also includes a static choke 60 within a receiver 68 on the stand 66, where the receiver 68 receives and supports the end of the main housing 57 with the nozzle 14 pointing downward. The choke 60 is in the form of a projection 69 within the main housing 57 that projects into the aperture 58 and the orifice 12 to restrict flow through the nozzle 14. By restricting the flow, this projection 69 automatically chokes the apparatus 2 to remove excess gas from the chamber 6 and to encourage the exhaust 49 to siphon, when the apparatus 2 is placed in the stand 66 with the nozzle 14 over the projection 69. However, should the body 4 be made of a material that might suffer damage of fracture when in contact with a conical projection 69, an alternative choke 60 can be used in place of this conical projection 69. For example, the choke 60 may be in the form of a rubber seal flat or a round or domed profile, against which the nozzle 14 seats, as shown in FIG. 24A. The stand has a hole 69A in (or close to) the middle of the seal or projection 69, allowing excess liquid to flow through, thereby avoiding the possibility that the apparatus 2 will being pushed off of the choke 60 by the force of the flowing liquid. The stand 66 can be placed, for example, in the base of a sink or tray for choking, to collect liquid flowing through the nozzle 14.

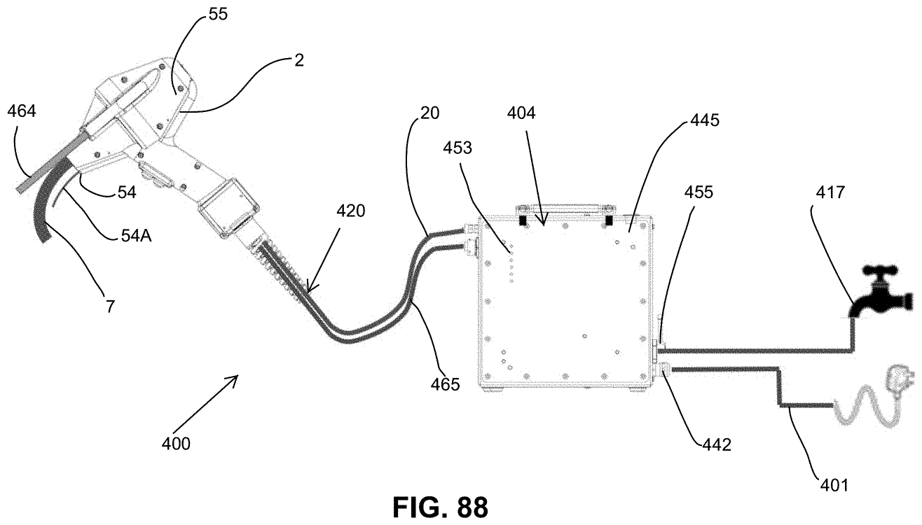

FIGS. 88-101 illustrate another embodiment of an apparatus 2 for delivering a stream of liquid containing bubbles and acoustic energy to a target, which is shown in FIG. 88 as part of an embodiment of a system 400 including the apparatus 2 with a control cabinet 404. The apparatus 2 of FIGS. 88-101 includes many features, both structural and operational, in common with the apparatus 2 described above with respect to FIGS. 1-11. Accordingly, such common features may be described herein with reference to the embodiment of FIGS. 1-11, rather than being fully described again, for the sake of brevity, and it is understood that the apparatus 2 of FIGS. 88-101 may include any of the features described herein with respect to the embodiment of FIGS. 1-11 unless explicitly noted otherwise. The apparatus 2 in FIGS. 88-101 has a hollow conical body 4 defining a chamber 6 configured as described above with respect to the embodiment of FIGS. 1-11. The body 4 has a base 11, with a rear wall 8 located at the base 11 and a substantially conical wall 10 extending forwardly away from the rear wall 8. The conical wall 10 terminates in an outlet nozzle 14 at an outlet 16 of the conical body 4, and the nozzle 14 includes an orifice 12 through which the liquid exits the chamber 6. The apparatus 2 also includes one or more vents 9 through which liquid containing any gas pockets can leave. In the embodiment of FIGS. 88-101, the apparatus 2 has a vent 9 configured to be at or near the uppermost point of the liquid in the chamber 6 during use in which the apparatus 2 is oriented so the nozzle 14 points downwardly, horizontally, or at some angle in between. In some embodiments, an outgasser may be used for the liquid supplied to the conical body 4 to reduce the build-up of gas within the conical body 4, such as an outgasser that uses a microporous filter, as described herein. The apparatus 2 may also include a controller 19 for controlling various operations of the apparatus 2 as described herein, such as the controller 19 shown schematically in FIGS. 1 and 20. The controller 19 may be part of the apparatus 2 itself, or may be located external to the apparatus 2 (e.g., in the control cabinet 404), or may include components located both internal and external to the apparatus.

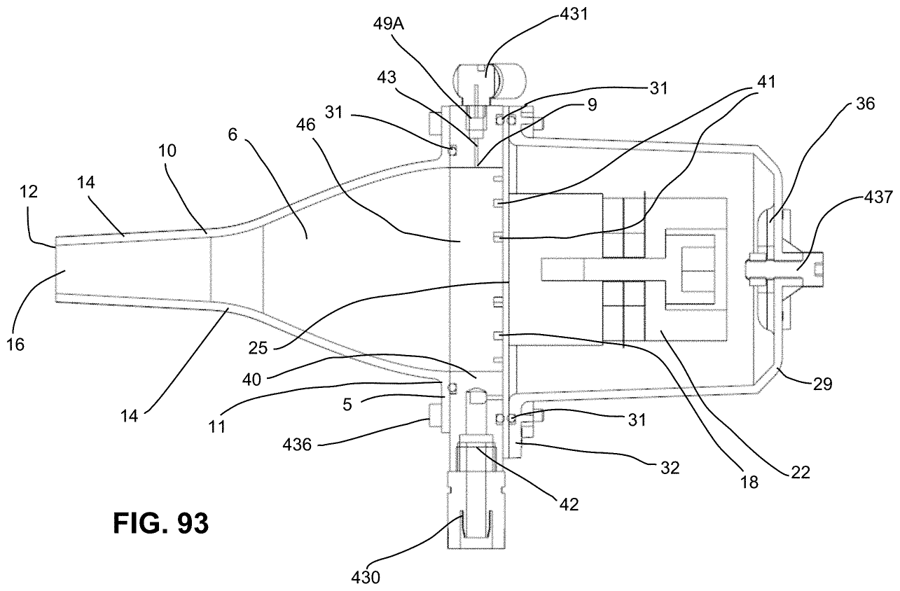

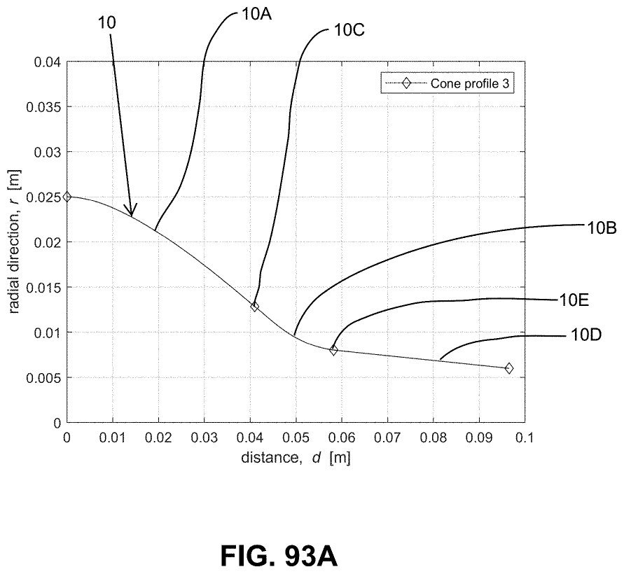

The body 4 in the embodiment of FIGS. 88-101 extends forwardly of the rear wall 8 to the nozzle 14, and the nozzle 14 may be integral with the end of the conical body 4, as similarly discussed with respect to the embodiment of FIGS. 1-11. The body 4 in this embodiment has a conical shape with concave and convex portions 10A, 10B and an inflection point 10C, and tapers in width from the base 11 to the nozzle 14, as also discussed herein with respect to FIGS. 1-11. The shape of the conical wall 10 in FIGS. 88-101 is shown in FIG. 93A and is identified as Profile 3, and this shape provides excellent acoustic performance that is similar to the performance of the cone of Profile 2 in FIG. 12, with greater ease of manufacturing using ABS or other molded plastic/polymer as described herein. In one embodiment, the conical wall 10 has a linear portion 10D that extends for at least a portion of the length of the nozzle 14, in which the conical wall 10 narrows in a constant linear manner (i.e., the inner and/or outer surfaces of the conical wall 10 appear linear in cross section). The ratio of the length of the linear portion 10D (measured along the central axis of the body 4) to the overall length of the body 4 in the embodiment of FIGS. 93 and 93A is about 0.4, and this ratio may be between 0.35 and 0.45 in another embodiment. The conical wall 10 in FIG. 93A has a linear portion 10D that extends from the end of the nozzle 14 (i.e., the front end of the body 4 or the orifice 12 in this configuration) to a linear start point 10E located at the end of the convex portion 10B. The end of the nozzle 14 may be considered a linear end point in this embodiment, and it is understood that the conical wall 10 may have a linear end point that is not located at the end of the nozzle 14 in other embodiments. In one embodiment, the slope of the wall of the linear portion 10D (in cross-section) is from 0.05 and 0.07 in absolute value, and the slope of the linear portion 10D in FIG. 93A is 0.06 in absolute value (specifically, −0.06 from left to right). Both the substantially conical wall 10 and the outlet nozzle 14 in FIGS. 88-101 are rotationally symmetric, i.e. circular, although other geometric shapes may be employed, such as other shapes described herein. The curved shape of the body 4 in this embodiment prevents a sudden change in cross section.

Additionally, in one embodiment, at least a portion, or the entirety, of the conical wall 10 is made of a single, molded piece that continuously narrows over its entire length, e.g., from the rear wall 8 to the end of the nozzle 14. In the embodiment of FIGS. 88-101, the entire conical body 4, including the rear wall 8 and the conical wall 10, are formed of a single molded piece, which may be formed from ABS or another plastic material (which may or may not include a fiber or other filler material). It is understood that the conical wall 10 may not continuously narrow at the same rate along the entire length, and the conical wall 10 in FIGS. 88-101 has a varying taper along the length. However, the conical wall 10 does not include any areas of constant or increasing diameter along the length of the conical wall 10 in the direction from the base 11 or the rear wall 8 to the end of the nozzle 14. This configuration improves manufacturing of the body 4 by facilitating molding of the conical wall 10, particularly by facilitating removal of the conical wall 10 from the mold piece and avoiding damage to the conical wall 10 in the process.

FIG. 93A depicts a sidewall profile (Profile 3) for the conical wall 10 of a body 4 in the same way as FIG. 12 discussed herein, with the “r” and “d” values representing the vertical and horizontal coordinates, respectively, in meters of the inner surface of the wall 10, assuming the axis of rotational symmetry is horizontal and lies on the axis defined by r=0. These profiles are defined as described above with respect to Profiles 1 and 2, and the basis data for Profile 3 is shown in Table 11 below.

TABLE 11
Basis Data for FIG. 93A
Cone profile 3

| d [m] | r [m] | S [m/m] |
|---|---|---|
| 0 | 0.025 | 0 |
| 0.041 | 0.0128 | −0.3949 |
| 0.0582 | 0.008 | −0.0602 |
| 0.0965 | 0.006 | −0.0602 |

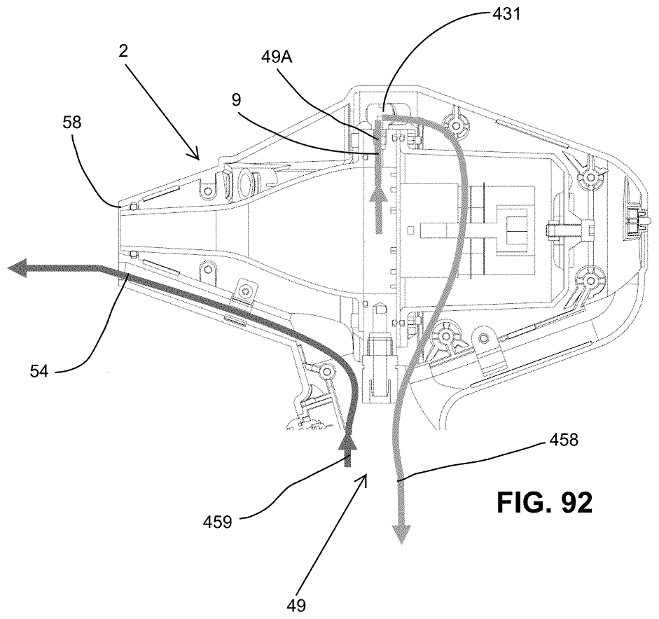

As with FIG. 12 above, it is understood that the two-dimensional profiles shown in FIG. 93A are symmetrically rotated about the length (d-axis) to define the shape of the body 4, and that these shapes do not include additional structure of the body 4 such as mounting structures (e.g., flange 5). It is also understood that the outer surface of the body 4 may be dimensioned using a constant wall thickness (e.g., 2 mm or other thickness described herein) based on the internal wall shape. The apparatus 2 further includes an acoustic transducer 22 positioned at or adjacent to the rear wall 8 and configured to introduce acoustic energy into the liquid within the chamber 6. The acoustic transducer 22 is mounted on the rear wall 8 in the embodiment of FIGS. 88-101, similarly to the embodiment of FIGS. 1-11. The controller 19 (such as shown schematically in FIGS. 1 and 20) controls the operation of the transducer 22 in any manner described herein. The transducer 22 is mounted on an outer surface of the wall 8 and extends over a substantial proportion of the surface area of the wall 8, as shown in FIGS. 92-93 and similarly described with respect to FIGS. 1-11. Alternatively, the transducer 22 may be mounted in a different configuration, such as shown in FIGS. 20-21 or described elsewhere herein.

The conical body 4 also includes one or more liquid inlets 18 located at or adjacent to the rear wall 8. In the embodiment of FIGS. 88-101, the apparatus 2 includes a manifold 40 located at the base 11 of the body 4 that includes a manifold body 45 having multiple ports 41 that operate as the inlets 18 of the body 4. The manifold 40 in this embodiment includes a manifold inlet 42 that is connected to a liquid supply conduit 20 in communication with a source/supply of liquid (as described herein) and one or more internal conduits 44 that extend from the manifold inlet 42 through the manifold body 45 to the plurality of ports 41 to distribute the liquid flow from the manifold inlet 42 to the ports 41, where the liquid enters the body 4. FIGS. 93-100 illustrate the manifold 40 of the embodiment of FIGS. 88-101 in greater detail. As shown in FIGS. 93-95, the manifold 40 is connected directly to the base 11 of the body 4 by connection of the manifold body 45 to a flange 5 that extends outward around the base 11. The flange 5 in this embodiment has a hexagonal shape, which permits the periphery of the flange 5 to be flush with (or otherwise not extend outwardly beyond) the top surface 435 of the manifold 40 with the body 4 joined to the manifold 40 in any rotational orientation. The manifold 40 is also connected directly to the rear wall 8 by connection of the manifold body 45 to the rear wall 8, such that a portion of the manifold body 45 is sandwiched between the rear wall 8 and the flange 5 of the base 11. The manifold body 45 includes a plurality of holes 47 that receive fasteners 30 for connection to the base 11 and the rear wall 8.

The manifold 40 in the embodiment of FIGS. 88-101 has a manifold inlet 42 and an exhaust outlet 49A at opposite sides of the manifold body 45, such as with the manifold inlet 42 at the bottom and the exhaust outlet 49A at the top. The manifold inlet 42 and the exhaust outlet 49A both have fittings 430, 431 that are connected to the manifold body 45 and are also connected to the liquid supply conduit 20 and the exhaust 49, respectively. The fittings 430, 431 may be connected to the manifold inlet 42 and the exhaust outlet 49A by threading in one embodiment, such that the fittings have male threading and are received in threaded openings in the manifold body 45.

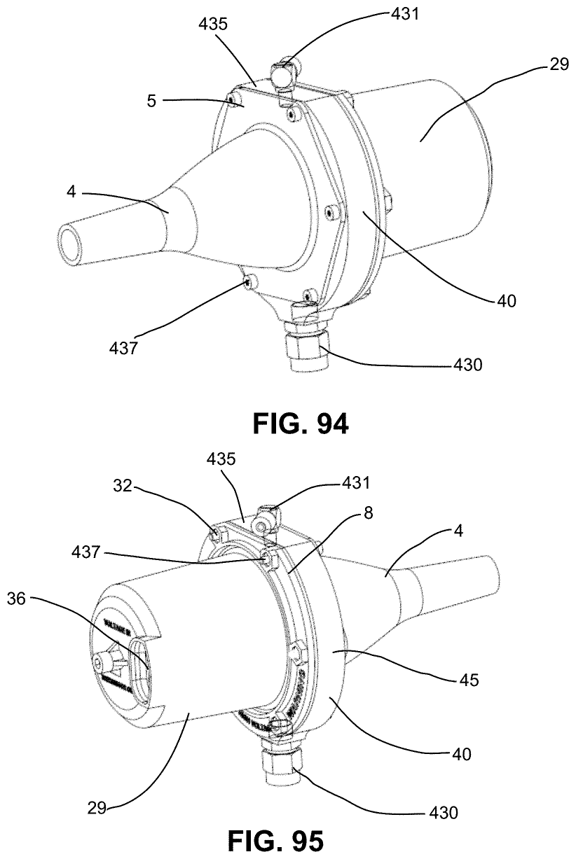

In the manifold 40 of FIGS. 88-101, the apparatus 2 is configured so that the inlet(s) 18 introduce the liquid into the chamber 6 in a flow direction that is radially inward with respect to the periphery of the conical wall 10 and parallel to the rear wall 8 and/or parallel to the face 25 of the transducer 22, similar to the manifold 40 of FIGS. 1-11. The manifold body 45 in this embodiment is formed in an annular shape with a central cavity 46, and the ports 41 of the manifold 40 are directed to introduce the liquid radially inwardly into the central cavity 46. In this configuration, the periphery 48 of the central cavity 46 partially defines the chamber 6, and the central cavity 46 exposes a portion of the rear wall 8. The internal conduits 44 in the embodiment of FIGS. 88-101 include two conduits 44 that branch in diverging directions away from the manifold inlet 42. The two conduits function as distribution chambers, with each having a plurality of ports 41. The ports 41 may be distributed at regular intervals around the central cavity 46 in one configuration. In the embodiment of FIGS. 1-11, the manifold 40 has two symmetrical conduits 44 each extending around slightly less than half of the periphery 48 of the central cavity 46 and having six ports 41 arranged into two sets of three ports, for a total of twelve ports. As described above, in the embodiment of FIGS. 5-8, the twelve ports 41 are distributed evenly at approximately 30° intervals around the central cavity 46. In the embodiment of FIGS. 94-99, each set of three ports 41 is centered at an angle of approximately 90° with respect to the adjacent sets and about 180° with the opposite set, and individual ports 41 of each set are distributed at intervals of approximately 22°. The use of multiple, evenly distributed inlets 18 reduces the amount of hydrodynamic turbulence within the chamber 6 and ensures a symmetric flow pattern to match the symmetric acoustic field generated by the transducer 22. In particular, the turbulence is reduced compared to a configuration having fewer, larger inlets 18 because smaller length-scales of turbulence have less energy and are more rapidly dissipated by the liquid viscosity.

The manifold 40 also includes a vent 9 positioned at the top end of the central cavity 46, similar to the manifold 40 of FIGS. 1-11. In this location, the vent 9 is configured to be positioned at or around the uppermost level of the liquid in the chamber 6 during use in which the apparatus 2 is oriented so the nozzle 14 points downwardly, horizontally, or at some angle in between. The vent 9 opens to the central cavity 46 and is thereby in communication with the chamber 6. The manifold 40 also includes a gas conduit 43 extending from the vent 9 through the manifold body 45 to the exhaust outlet 49A.

It should be noted that the embodiments of the manifolds 40 shown in FIGS. 1-11 and in FIGS. 90-101 are only example embodiments. Additional features may be included in other embodiments, which may potentially improve functionality for the manifold 40, as described elsewhere herein, e.g., with respect to FIGS. 1-11. Additionally, manifolds 40 as described herein may be used in connection with any embodiment described herein. It is understood that in some embodiments herein, such as those in FIGS. 20-21 and 31-32, the drawings are depicted schematically, and the manifold 40 (or other liquid inlet 18) is not illustrated for the sake of simplicity. Nevertheless, such embodiments are contemplated for use with a manifold 40 or other liquid inlet(s) 18.

The manifold 40 in the embodiment of FIGS. 88-101 provides improved manufacturing capability, as all of the internal passages (including the conduits 44, the ports 41, the vent 9, the manifold inlet 42, the exhaust outlet 49A, and potentially the central cavity 46) can be manufactured by milling. In particular, the conduits 44 and ports 41 can be manufactured by milling because they are open on the rear surface of the manifold 40 and are covered and sealed by the rear wall 8. This greatly decreases the cost and increases the ease of manufacturing the manifold 40.

The rear wall 8 in FIGS. 88-101 is positioned at the base 11 of the body 4 and defines at least a portion of the rear of the chamber 6, similar to the embodiment of FIGS. 1-11. The rear wall 8 comprises a plate as described herein. In one embodiment, the acoustic transducer 22 is positioned in contact with the rear wall 8 and may be mounted on the rear wall 8 or otherwise connected to the rear wall 8. The transducer 22 and the rear wall 8 may be mounted in any configuration described herein, such as those shown in FIGS. 1-11. The rear wall 8 in FIGS. 88-101 is connected to the rear side of the manifold body 45 as described herein and covers the central cavity 46 of the manifold 40 to define the rear end of the chamber 6. In this configuration, the liquid inlets 18 are directed parallel and adjacent to the portions of the rear wall 8 exposed within the central cavity 46 of the manifold 40. An inner housing 29 is also connected to the rear side of the rear wall 8 to enclose the transducer 22, using the same fasteners 30 connecting the rear wall 8 and the body 4 to the manifold 40, e.g., as shown in FIGS. 93-95. These fasteners 30 may be in the form of bolts that are torqued when inserted, in order to connect the components together sufficiently securely without clamping the rear wall 8 too tightly, which can affect vibration of the rear wall 8.

The body 4 in FIGS. 88-101 has a length L of 96.5 mm, a diameter D1 at the base 11 of 50 mm, a diameter D2 at the nozzle 14 of 12 mm, and a wall thickness of 2 mm, and the inflection point 10C is located 52 mm from the base 11 of the body 4, with a diameter D3 at the inflection point 10C of approximately 17 mm. The body 4 also has an internal chamber 6 volume V of approximately 83,490 mm³ or 83.49 ml, and the rear wall 8 has a diameter of 92.5 mm, with an area A1 having a diameter of 50 mm and a surface area of 1963.5 mm² being exposed to the chamber 6. It is understood that the dimensions D1, D2, D3, V, and A1 are defined in the same manner with respect to this embodiment as with respect to the embodiment of FIGS. 1-4 as described herein. In other embodiments, the body 4 may have a structural configuration with dimensions that are within +/−10% of these values or +/−5% of these values. The apparatus 2 shown in FIGS. 88-101 is configured for producing a 135 kHz acoustic frequency, and these dimensions are based on that. The information in Table 2 discussed herein can guide minimum and maximum nozzle 14 diameters, and the dimensions of the body 4 may be scaled and adjusted based on various parameters as described herein.

In the embodiment of FIGS. 88-101, the ratio of the exposed area A1 of the rear wall 8 to the area A2 of the front surface of the transducer 22, and the ratio of the volume V of the chamber 6 to the exposed area A1 of the rear wall 8, may correspond to embodiments described elsewhere in the specification. For example, the ratio of the exposed area A1 of the rear wall 8 to the area A2 of the front surface of the transducer 22 is from 1.4:1 to 2.2:1, or from 1.5:1 to 2.1:1, or from 1.6:1 to 2.0:1, or from 1.7:1 to 1.9:1, and in another embodiment, the ratio of A1:A2 is about 1.8:1. As another example, the ratio of the volume V of the chamber 6 to the exposed area A1 of the rear wall 8 is from 38 mm³:1 mm² to 50 mm³:1 mm², or from 39.5 mm³:1 mm² to 48.5 mm³:1 mm², or from 41 mm³:1 mm² to 47 mm³:1 mm², or from 42.5 mm³:1 mm² to 45.5 mm³:1 mm², and in another embodiment, the ratio of V:A1 is about 44 mm³:1 mm².

The apparatus 2 in FIGS. 88-101 has a venting system that includes one or more vents 9 in communication with the chamber 6 for removal of gas, positioned to be located at or near the uppermost point of the liquid in the chamber 6 during use (e.g., with the nozzle 14 pointed sideways and/or downward, as described herein with respect to FIGS. 1-11. The venting system in this embodiment also includes an exhaust 49 in communication with the vent(s) 9, via connection with the exhaust outlet 49A of the manifold 40. The exhaust 49 in this embodiment is in the form of tubing and is configured to enable creation of a siphon effect to continuously remove gas from the liquid in the chamber 6 during use. The exhaust 49 in FIGS. 88-101 extends to a pump 39 as similarly shown in FIG. 46 and described in greater detail below, which pumps gas and liquid through the exhaust 49 to the pump 39 and then to an outlet 54 of the exhaust 49. The exhaust 49 in this embodiment may be considered to be part of an exhaust system including the vent 9, the pump 39, and any conduits forming the exhaust 49 and extending to the outlet 54. In this configuration, the use of a choke 60 is not necessary, as the vent 9 and the exhaust 49 remove gas from the chamber 6 to allow the chamber 6 to fill completely. The outlet 54 in this embodiment is positioned proximate the nozzle 14, as shown in FIG. 46, which can create a Venturi effect in the exhaust 49 to assist in drawing liquid and gas through the vent 9 and the exhaust 49. In one embodiment, the pump 39 may be operated continuously during operation, and in another embodiment, the pump 39 may be selectively operated when gas needs to be removed from the chamber 6 (i.e., after the exhaust 49 is filled with liquid without significant bubbles). The use of transparent tubing in the exhaust 49 may assist with this determination. If there is no liquid flow observed to come from the exhaust 49 at any point during operation, this is also an indication that activation of the pump 39 is required.

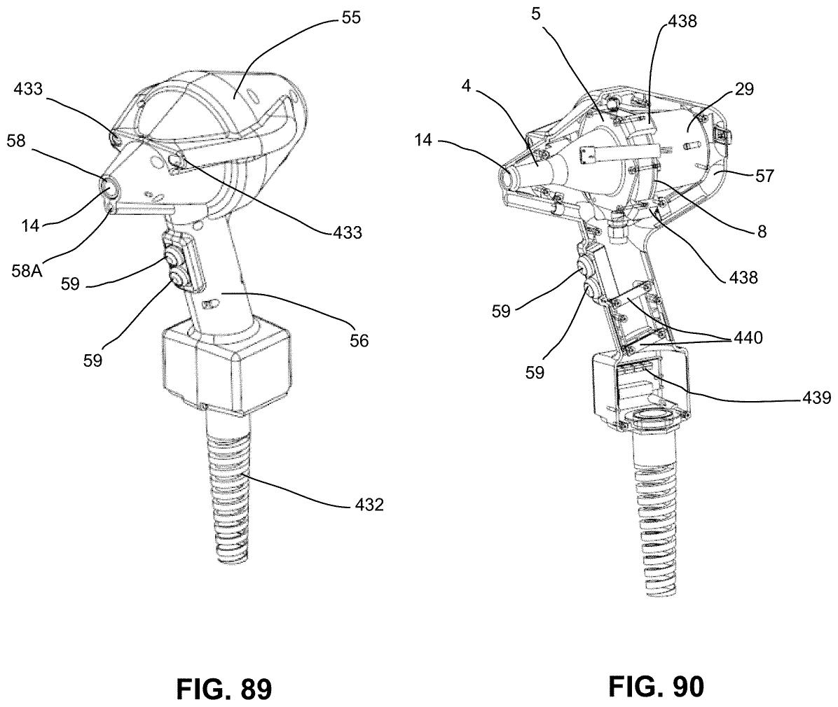

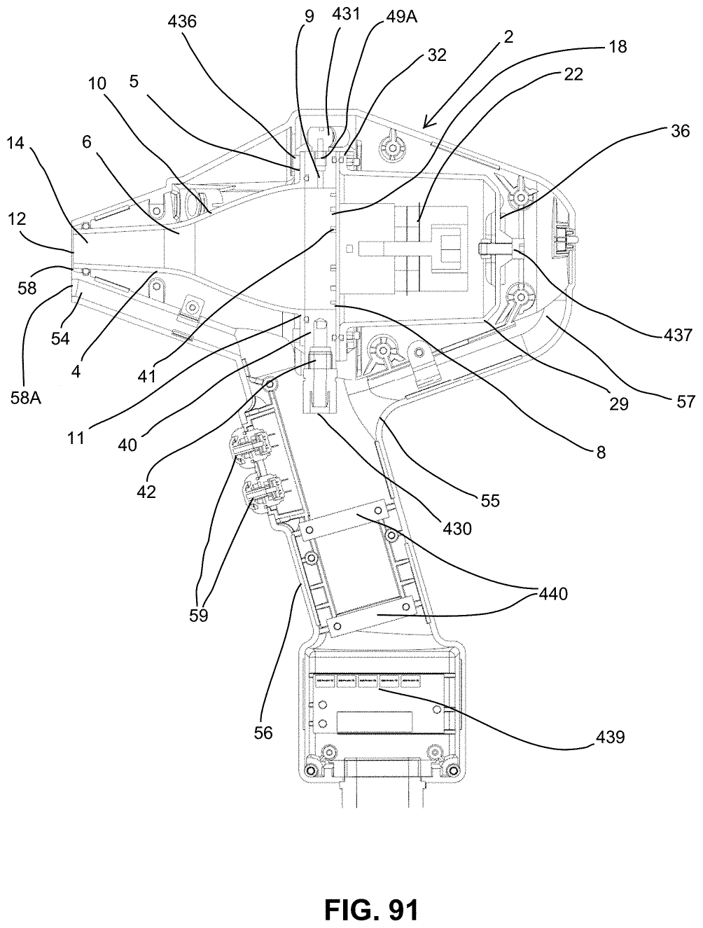

The apparatus 2 of FIGS. 88-101 is configured as a self-contained handheld delivery device and includes a casing 55 configured to facilitate handheld use, similar to the embodiment of FIGS. 1-11. The casing 55 in this embodiment is shown in FIGS. 88-92, and includes a handle 56 and a main housing 57 with two apertures 58, 58A at one end. The casing 55 may be formed in various manners, including by use of two half-pieces that may be separate or configured as a clamshell piece, which are joined together by fasteners. FIGS. 88-89 illustrate the casing 55 with two half-pieces assembled together, and FIG. 90 illustrates the casing 55 with one of the half-pieces removed. The handle 56 is connected to the main housing 57 and is configured for gripping by the operator to move and direct the apparatus 2. The apparatus 2 in this embodiment also includes two buttons 59 located on the handle 56, which are used to initiate the liquid flow and the transducer 22, respectively. The handle 56 also encloses electrical components, i.e., to receive input from the buttons 59 and operate LEDs on the casing 55, as well as to interact with other components of the apparatus. For example, a printed circuit board (PCB) 439 is located within the base of the handle 56 in one embodiment, as shown in FIGS. 90-91. Clamps 440 for holding in place the conduits that extend through the umbilical 420 may also be located within the handle 56, as also shown in FIGS. 90-91. It is noted that various components of the apparatus that are contained within the casing 55 may be held in place by fasteners, bonding, and/or sandwiching the components between the two pieces of the casing 55, among other structures.

The main housing 57 encloses the transducer 22, the body 4, the rear wall 8, and the manifold 40, among other components, and these components are mounted fixedly within the main housing 57. The body 4 is positioned such that the nozzle 14 is located at one of the apertures 58 of the main housing 57, and the stream 7 passes through the aperture 58. The outlet 54 of the exhaust 49 is located at the other aperture 58A, which is adjacent to and below the aperture 58 through which the stream 7 passes. The manifold inlet 42 extends from the main housing 57 into the handle 56, and the liquid supply conduit 20 extends upward through the handle 56 to connect to the manifold inlet 42. The exhaust 49 also passes downwardly from the main housing 57 through the handle 56 to exit the apparatus 2 (to the pump 39) and also back up through the handle 56 (from the pump 39) to the outlet 54. The liquid supply conduit 20 and the output/return conduits 458, 459 of the exhaust 49 may be consolidated within an umbilical 420 extending from the control cabinet 404 to the apparatus as described elsewhere herein, which also includes one or more electrical conduits 465. The umbilical 420 in this embodiment has a strain relief portion 432 located where the umbilical 420 meets the casing 55.

The apparatus 2 in FIGS. 88-101 further has an inner housing 29 enclosing the transducer and associated components. The housing 29 has flanges 32 for connection to the rear wall 8 and the manifold 40, e.g., by fasteners 30, as well as electrical shielding for safety and to avoid electromagnetic leakage, as described elsewhere herein. In one embodiment, the housing 29 may be formed of plastic with an electromagnetic shielding paint applied to the plastic material for shielding. Other components may also be configured to resist electromagnetic leakage as described herein. O-rings or rubber washers or gaskets 31 are located between the manifold 40 and the body 4 and the rear wall 8, and also between the rear wall 8 and the inner housing 29, to resist liquid leakage. The inner housing 29 further has a power port 36 to permit one or more electrical conduits to enter the inner housing 29 for powering components such as the transducer 22, as shown in FIGS. 93 and 95. A potting compound or other sealing material may be applied to the power port 36 for sealing against water ingress, which may also provide a strain relief effect on the conduit(s). The inner housing 29 also may have a ground connection 437, as shown in FIGS. 93 and 95.

The body 4, the manifold 40, the rear wall 8, the transducer 22, and the inner housing 29 in the embodiment of FIGS. 88-101 are all connected together to form a unit that is contained within the main housing 57 of the casing 55. These components may be held within the casing 55 using various engagement structures, including permanent or releasable connectors, bonding material, or bracing structures. The apparatus 2 of FIGS. 88-101 uses isolation mounts 438 made of rubber or other resilient material for holding this unit in place within the casing 55. These isolation mounts 438 engage the unit around the periphery of the manifold 40 and also engage the inner surfaces of the main housing 57 of the casing 55. The isolation mounts 438 in this configuration both hold the components in place and isolate the unit (especially the rear wall 8 and the transducer 22) vibrationally from the rest of the apparatus 2.

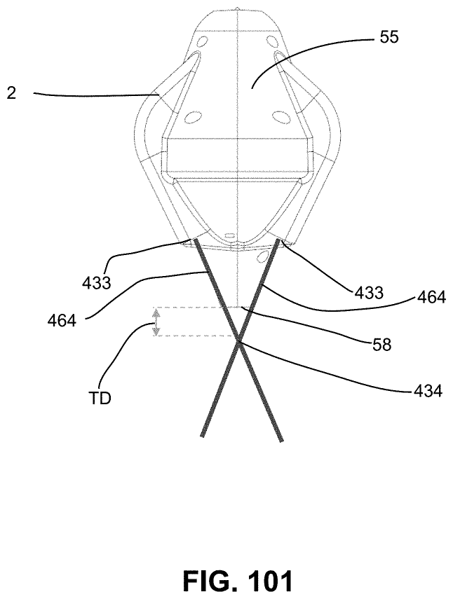

The apparatus 2 in FIGS. 88-101 further includes a range finding or estimating mechanism, which in this embodiment includes two laser emitters 433 located on opposite sides of the casing 55. FIG. 101 schematically illustrates the use of the laser emitters 433 for determining the proper range from the target to the nozzle 14 for maximum effectiveness of the apparatus 2, and FIG. 88 also schematically illustrates the beams 464 of the emitters 433. The laser emitters 433 are contained within cavities on the exterior of the casing 55 in this embodiment, and may also include light filters (not shown). In this embodiment, the beams 464 of the laser emitters 433 converge at a target point 434 located at a target distance TD from the aperture 58 of the casing 55. This allows the operator to visually confirm the proper range of the apparatus 2. In one embodiment, the target distance TD may be about 20 mm. In one embodiment, the laser emitters 433 are automatically activated (e.g., by the controller) any time the transducer 22 is activated to produce acoustic energy. In other embodiments, the laser emitters 433 may be manually activated (e.g., by a dedicated button press) and/or automatically activated in a different manner, e.g., any time the liquid flow through the nozzle 14 is activated.

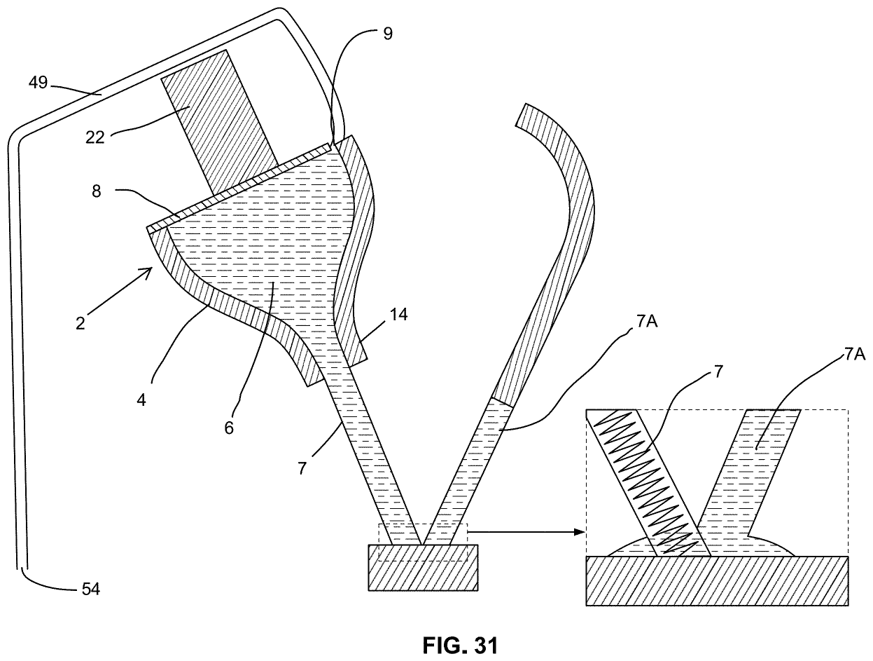

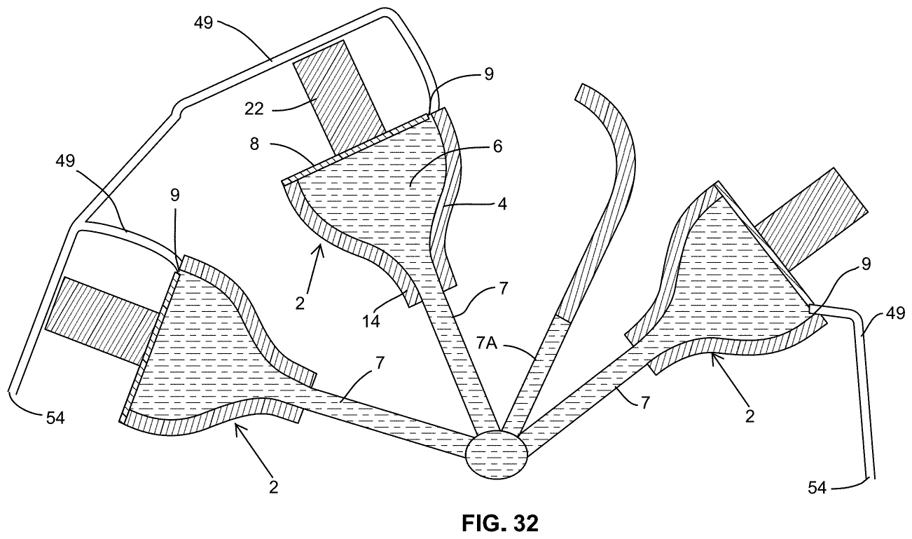

In another embodiment, an apparatus 2 as described herein may be used to generate a liquid stream 7 that conducts acoustic energy to the target without bubbles of a sufficient size and number to disrupt the acoustic waves, and a second stream 7A including bubbles (without acoustic energy) may be supplied, so that the two streams 7, 7A meet at the target. FIGS. 31 and 32 illustrate embodiments that use this configuration, with FIG. 32 including multiple apparatuses 2 producing streams 7 with acoustic energy. In this embodiment, the streams combine so that the acoustic energy in the first stream 7 energizes the bubbles in the second stream 7A to produce beneficial effects as described herein. The transducer 22 in this embodiment may be operated continuously or in a pulsed fashion, such as by controlling the operating parameters (e.g., amplitude, frequency, tₒₙ) of the transducer 22 to avoid production of bubbles and/or by using liquid that is completely degassed. The second stream 7A may include bubbles produced by various mechanisms, including a bubble generator, and may include a steady stream of bubbles rather than swarms of bubbles. The bubbles in the second stream 7A should be sized to be predominantly resonant bubbles and to avoid unwanted bubbles, based on the frequency of the energy from the transducer 22, or vice-versa. In the system shown in FIG. 32, two or more apparatuses 2 may share a single exhaust 49 (i.e., a single tube) and/or a single liquid supply conduit 20, for example, if space restraints require. It is noted that the use of separate tubes for the exhaust 49 can avoid a large bolus of bubbles from one apparatus 2 stalling the siphon of the other apparatus 2.

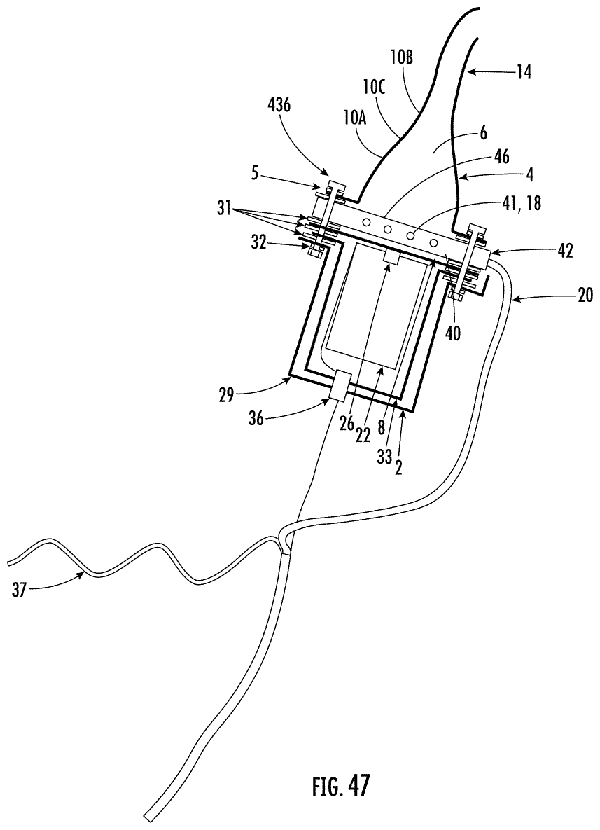

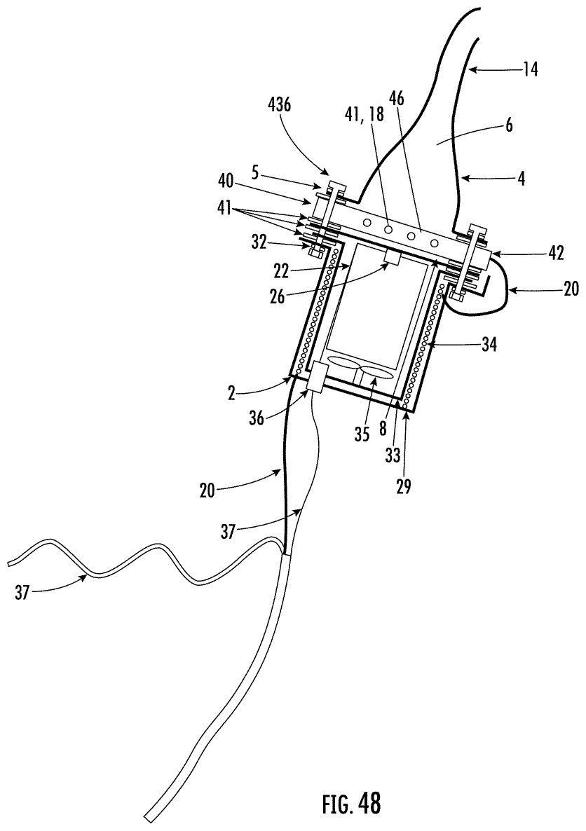

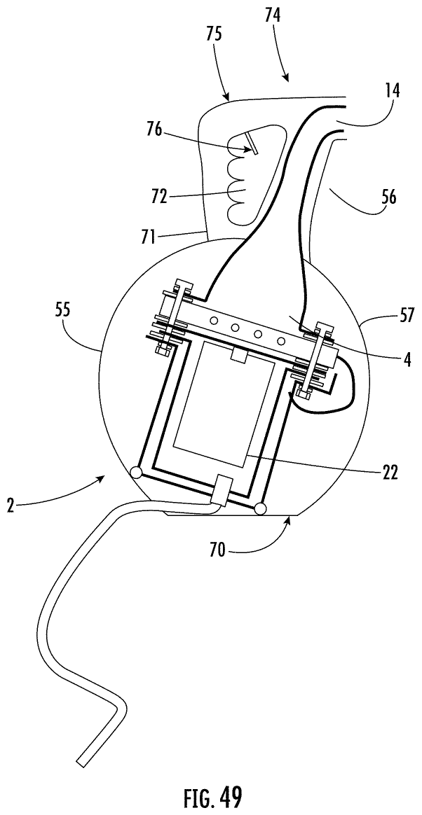

FIGS. 47-52 illustrate another embodiment of an apparatus 2 that includes many components and features in common with the apparatus 2 in FIGS. 1-11 described herein. As such, these similar components and features in the embodiment of FIGS. 47-52 will be described and referenced with similar reference numbers. Additionally, some or all such common components and features may not be re-described herein in detail, except with respect to significant differences from those embodiments described elsewhere. FIGS. 47 and 48 illustrate two different configurations for the internal components of the apparatus 2, which include a body 4, a rear wall 8, a manifold 40 positioned between the body 4 and the rear wall 8 and coupled to both, and a transducer 22 connected to the rear wall 8 and enclosed by an inner housing 29. It is noted that the apparatuses of FIGS. 47 and 48 do not include any vent 9, exhaust 49, or choke 60. FIGS. 49-52 illustrate different embodiments of casings 55 that can be used with the apparatuses 2 of FIGS. 47-48.

The embodiments of FIGS. 47-48, as illustrated, use a manifold 40 with a liquid distribution system configured similarly to that of the manifold 40 in FIGS. 1-11. It is understood that a different manifold 40 configuration may be used in another embodiment. The manifold 40 in FIGS. 47-48 includes a manifold inlet 42 that is connected to a liquid supply conduit 20 in communication with a source of liquid and one or more internal conduits (not shown) that extend from the manifold inlet 42 through the manifold body 45 to a plurality of ports 41 that operate as inlets 18 where the liquid enters the body 4. The manifold 40 also has a central cavity 46 that partially defines the chamber 6 and exposes a portion of the rear wall 8, and the ports 41 are distributed evenly around the central cavity 46.

The body 4 in FIGS. 47-48 is configured to be oriented with the length or axial direction in a vertical or predominantly vertical direction during use, and the nozzle 14 is bent in a curvilinear manner to direct the flow of liquid in a desired direction. In one embodiment, the nozzle 14 is bent at least 90°, and in another embodiment, the nozzle 14 is bent at least 120°. The turn 104 of the nozzle 14 must be gradual enough so that the acoustic waves are propagated down the nozzle 14 to the stream 7 and not reflected back to the transducer 22. The conical wall 10 of the body 4 may have a shape and contour as described herein, with concave and convex portions 10A, 10B, and may be made from any material described herein. The body 4 also has a flange 5 for connection to the manifold 40, such as by fasteners 30 in the form of bolts, with O-rings or rubber washers or gaskets 31 between the components to resist liquid leakage. The housing 29 may similarly have flanges 32 for connection to the rear wall 8, the manifold 40, etc., as well as internal electrical shielding 33 for safety and to avoid electromagnetic leakage. Resisting electromagnetic leakage is further improved if other components such as the rear wall 8, the outer casing 55, the handle 56, the main housing 57, the body 4, and/or the nozzle 14 are made from a conductive shielding material, e.g., metal. The housing 29 further has a power port 36 that is connected to an electrical supply 37 for powering components such as the transducer 22 and a cooling fan 35 (described below) if present.

The embodiment of FIG. 48 further includes internal cooling components to cool the transducer 22, including one or more cooling coils 34 and/or a cooling fan 35. The cooling coils 34 wrap around the transducer 22 and are connected to the liquid supply conduit 20 and the manifold inlet 42, so that the liquid entering the manifold 40 absorbs heat from the transducer 22 prior to entering the manifold and the chamber 6. In FIGS. 47-50, the cooling coils 34 wrap between the inner housing 29 and the electrical shielding 33. If the electrical shielding 33 is metal, it can provide electrical shielding and resistance to electromagnetic leakage as described above, as well as thermal conduction, to allow the cooling coils 34 to better remove heat from the transducer 22. Outside of this, the inner housing 29 can be electrically insulating (e.g., plastic) to provide electrical safety. The cooling fan 35 in this embodiment is a low voltage fan that may be electrically powered through the power port 36.

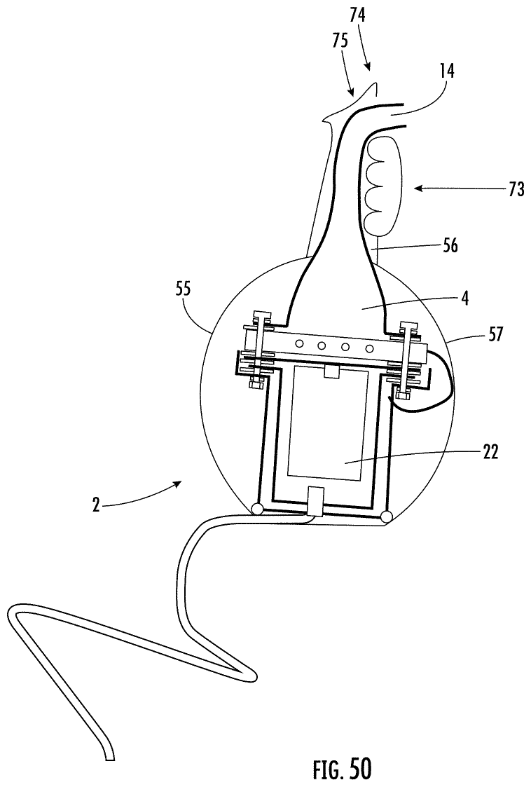

FIGS. 49-50 illustrate casings 55 configured for use with the apparatuses 2 of FIGS. 47-48, each having a bulbous main housing 57 with a flat base 70 configured for resting on a surface and a handle 56 located above the main housing 57. These casings 55 enclose the other components of the apparatus 2, which may also include the controller (not shown). The nozzle 14 extends through the handle 56 in both embodiments and out through an aperture (not shown). The handle 56 in FIG. 49 includes a gripping portion 71 extending off of the handle to define a gripping slot or hole 72 for receiving a portion of an operator's hand. The handle 56 in FIG. 50 is configured for the operator to grip directly with his/her hand (shown as 73). Reference numbers 74 and 75 show the locations of the LEDs for providing output to the operator and the control buttons, respectively. The apparatus 2 of FIG. 49 may have a control trigger 76 within the gripping hole 72 for activating one of the liquid flow and the transducer 22, with a control button 75 for activating the other.

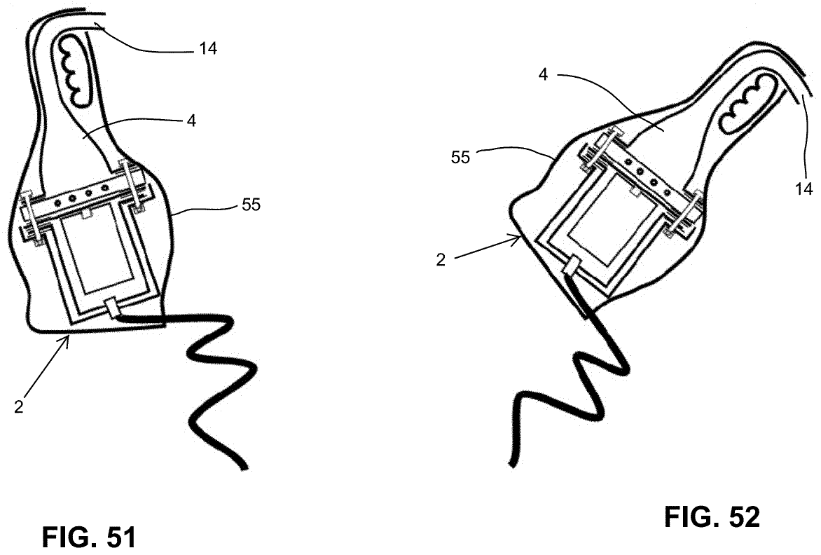

As described herein, the apparatuses 2 in FIGS. 47-50 do not require venting or choking mechanisms, as gas in the chamber 6 can escape upward through the nozzle 14 as liquid flows into and out of the chamber 6. It is understood that if the apparatus 2 is tilted forward by more than about 60°, for example to direct the stream onto a horizontal surface, some gas may become trapped within the chamber 6. This can degrade performance by reducing the amplitude of the acoustic field that can be generated in the body 4. Accordingly, the length or axial direction of the body 4 or the chamber 6 may be slightly offset from vertical, but nevertheless close to vertical, to allow gas to rise from the body 4 to enter the faster-flowing region of the nozzle 14 and be flushed out into the stream 7, even when the apparatus 2 is tilted to direct the stream onto a horizontal surface. In one embodiment, the degree of bend or curve in the turn 104 of the nozzle 14 may exceed 90° in one embodiment or may exceed 120° in another embodiment. FIGS. 51-52 illustrate one embodiment where the axial direction of the body 4 is oriented to be nearly vertical but angled slightly rearward at a position where the nozzle 14 is directed horizontally, i.e., parallel to the resting surface, as shown in FIG. 51. The bend in the nozzle is greater than 90° in this embodiment. In this configuration, the axial direction of the body 4 is oriented to be somewhat vertical in a position where the nozzle 14 is directed forward and downward, as shown in FIG. 52. In one embodiment, the apparatus 2 may be configured to have at least one position where the axial direction of the body 4 is oriented to be primarily vertically upward (i.e., less than 45° from the upward vertical direction) while the direction of the nozzle 14 is primarily vertically downward (i.e., less than 45° from the downward vertical direction). Additionally, in one embodiment, the apparatus 2 may be configured so that the nozzle 14 may be positioned directly vertically downward while the axial direction of the body 4 is oriented to be angled away from the upward vertical direction by no more than about 60°. The configuration shown in FIGS. 51-52 therefore permits gas to be vented from the chamber in either position (FIG. 51 or FIG. 52) without the need for a choke and/or vent, and allows use without trapping gas in the chamber 6.

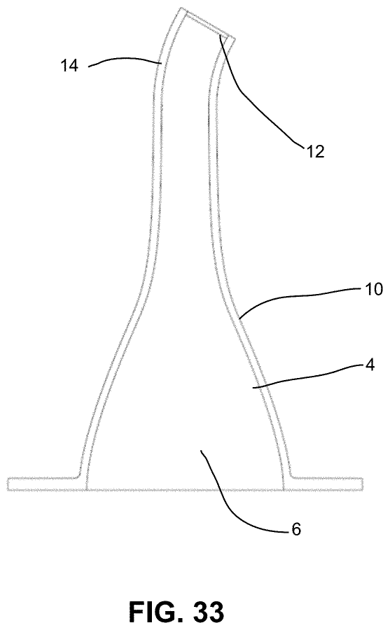

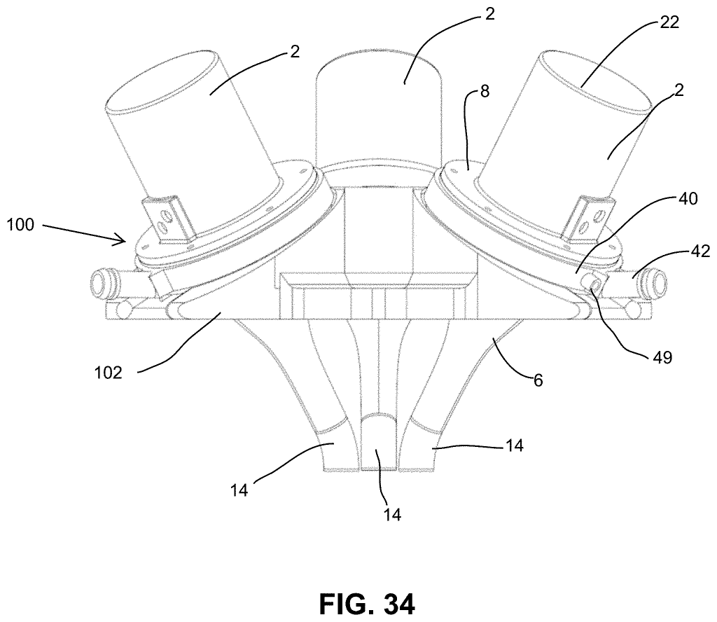

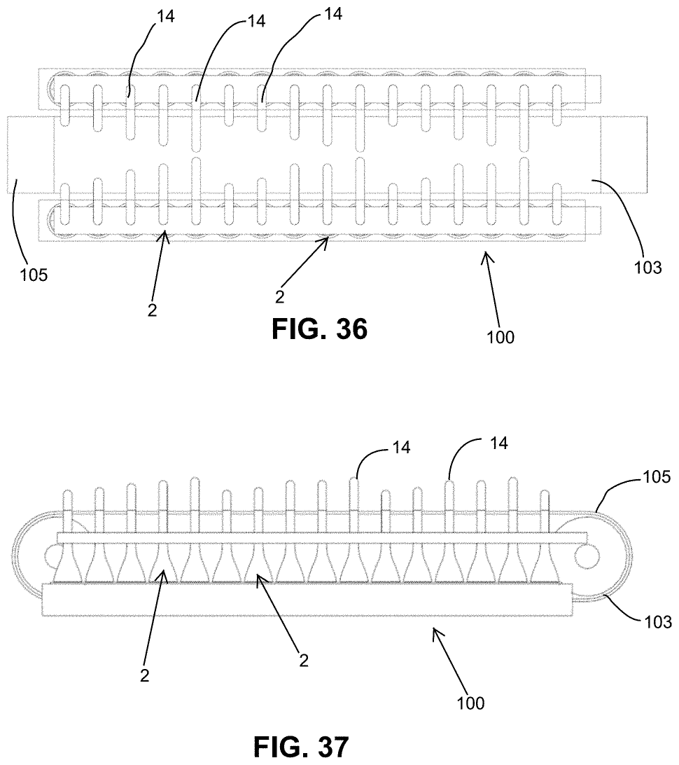

The apparatus 2, in various embodiments, may be combined with other apparatuses 2 into a system 100 that may be used for applying streams 7 to multiple targets in succession. FIGS. 33-34 illustrate one embodiment of a system 100 that uses multiple apparatuses 2 (three in this specific embodiment) that have nozzles 14 positioned immediately adjacent to each other, such that the multiple streams produce a wide curtain of liquid including bubbles and acoustic energy as described herein. The system 100 in FIGS. 33-34 is configured to use an apparatus 2 having a transducer 22, a rear wall 8, and a manifold 40 as described herein and shown in FIGS. 1-11, with a conical body 4 that is generally the same as in FIGS. 1-11, but with a differently configured nozzle 14. As shown in FIG. 34, the system 100 includes a support frame 102 that is configured to hold and support the apparatuses 2 with the length or axial direction of the bodies 4 at 3-dimensional angles to each other (e.g., 45°), so the nozzles 14 converge together. The body 4 used for the apparatuses 2 in FIG. 34 is illustrated in greater detail in FIG. 33, and the nozzle 14 is curved and elongated slightly relative to the nozzle 14 in FIGS. 1-4. The bodies 4 of the apparatuses 2 in FIG. 34 are oriented so that the nozzles 14 all point directly downward. While not shown in FIG. 34, a liquid supply conduit 20 may be connected to the manifold inlet 42 of each apparatus 2, and a tube forming the exhaust 49 can be connected to the gas conduit 43 of each apparatus 2. The apparatus 2 may or may not include any casing as described herein. The curtain of liquid produced by the multiple apparatuses 2 in FIG. 34 can be directed at a single target, or may be configured to be directed at multiple targets through relative movement. For example, the support frame 102 may be fixed and the nozzles 14 directed at a moving support (not shown) that carries multiple targets through the streams 7, such as a conveyor that moves along the viewing direction in FIG. 34. FIGS. 36-40 illustrate examples of such a conveyor 103. A series of systems 100 as shown in FIG. 34 can be positioned along the length of the conveyor 103 in one embodiment, to create a plurality of rows of streams 7. As another example, the support frame 102 may be moveable to move the streams 7 across a platform (not shown) holding multiple targets.

In a system 100 with multiple apparatuses as shown in FIG. 34, choking (if necessary) can be accomplished using any choke 60 configuration described herein. In another embodiment, a single choke (not shown) may be configured to operate on all of the nozzles 14 in a row, such as a row of conical stops, balls or plates spaced the same as the nozzles 14. When a series of systems 100 as shown in FIG. 34 are used, this multiple choke can be moved sequentially to each system 100 to restrict the rows of nozzles 14 sequentially.

FIGS. 35-39 illustrate additional embodiments of systems 100 that include multiple apparatuses 2 that are configured to direct streams 7 of liquid at a conveyor 103 or other moveable support, where the streams 7 include bubbles and acoustic energy as described herein. An example of an apparatus 2 used in FIGS. 35-38 is shown in FIG. 35, and may use a transducer 22, rear wall 8, and manifold 40 as shown in FIGS. 1-11. The conical body 4 in FIG. 35 has a “goose neck” configuration, with the length or axial direction of the body 4 oriented vertically such that the liquid flows upwardly out of the chamber 6 and the transducer 22 is directed upwardly, with the nozzle 14 making a complete or nearly complete (e.g., 180° or close to 180°) turn 104 to direct the stream 7 downwardly. The conical wall 10 in this embodiment is significantly shorter in length, and the nozzle 14 is significantly longer, as compared to the embodiment of FIGS. 1-11. The nozzle 14 may be formed as a cylindrical tube having a length of 5-50 cm. The turn 104 of the nozzle 14 must be bent or curved gradually enough so that the acoustic waves are propagated down the nozzle 14 to the stream 7 and not reflected back to the transducer 22. The material of the body 4, including the nozzle 14, is configured as described herein to ensure that coupled modes are set up between the material of the body 4 and nozzle 14 wall, and the liquid. These allow the acoustic wave to propagate the length of the nozzle 14, and couple into the stream 7 despite the pressure-release water-to-air boundary condition that exists at the perimeter of the stream 7 for the acoustic field in the stream 7. In this configuration, the use of a vent 9 and a choke 60 is not necessary, as gas will escape upward from the chamber 6 and exit through the nozzle 14 as the chamber 6 is filled, if sufficient pressure and flow speed exists.

The system 100 in FIGS. 36-38 uses a plurality of apparatuses 2 with goose neck nozzles 14 as shown in FIG. 35 positioned along a conveyor 103, with different apparatuses 2 having nozzles 14 that extend different distances laterally (i.e., perpendicular to the length or axial direction of the body 4). This places the nozzles 14 of adjacent apparatuses 2 at staggered lateral locations relative to the conveyor 103, and the system 100 is configured so that the streams 7 of the various apparatuses 2 cover the entire width of the conveyor 103. As shown in FIGS. 36-38, the system 100 includes apparatuses 2 with five different types of nozzles 14 having five different lateral extensions, positioned in matching pairs on opposite sides of the conveyor 103, thereby covering ten separate locations across the width of the conveyor 103. It is understood that the nozzles 14 extending further in the lateral direction may also extend higher in the vertical direction in order to ensure a gradual curvature of the nozzle 14.

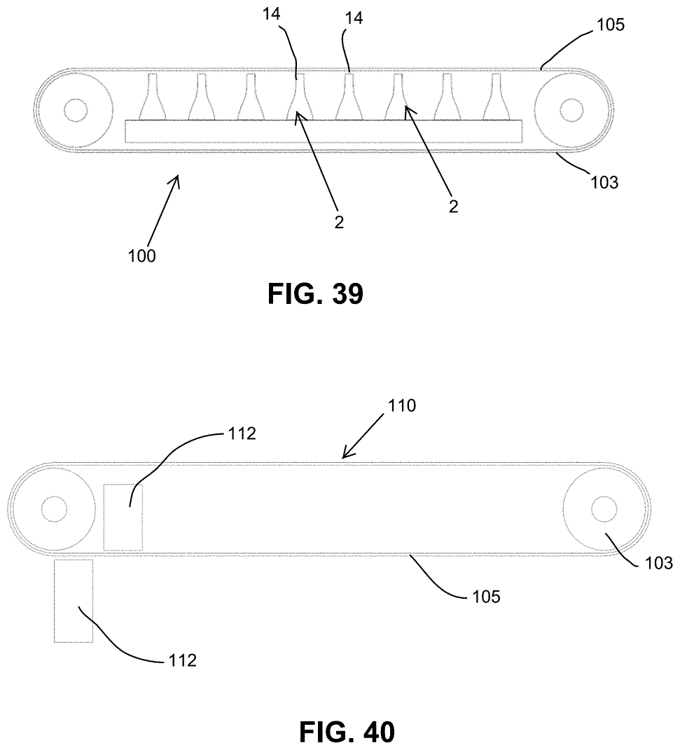

FIG. 39 illustrates another embodiment of a system 100 that may be used independently or in conjunction with the systems 100 in FIGS. 34 and 36-38. The system 100 of FIG. 39 uses a plurality of apparatuses 2 as described herein with nozzles 14 pointing upward to direct the stream 7 upward to contact the underside of the belt 105 of the conveyor 103. In this configuration, a belt 105 having a permeable configuration (e.g., mesh) may be used to permit the apparatuses 2 of FIG. 39 to introduce streams 7 to the underside of any articles positioned on the conveyor 103. These apparatuses 2 also do not require vents 9 or chokes 60, as gas can escape from the chambers 6 upward through the nozzles 14.

The systems 100 in FIGS. 33-39 can be used in a large-scale cleaning system in one embodiment, such as for cleaning large amounts of produce or other potentially fragile products. Such products are transported, e.g., by the conveyor 103, through the streams 7 from the apparatuses 2 to thoroughly clean the products. The systems 100 can also be used to clean the belt 105 itself during operation, reducing the down-time required to perform routine cleaning of the belt 105. Currently, many cleaning systems in uses such as food production and processing stop the belt 105 for periodic (e.g., daily) cleaning, which reduces productivity. Reducing or eliminating this down-time can therefore greatly increase productivity.

FIG. 40 illustrates another embodiment of a system 110 that may be used independently or in conjunction with the systems 100 in FIGS. 34 and 36-39. The system 110 in FIG. 40 includes two cleaning apparatuses 112 positioned on the return loop of a conveyor 103, in order to clean the upper and lower surfaces of the belt 105. The cleaning apparatuses 112 may be apparatuses 2 as described herein or a different type of ultrasonic cleaning apparatus, or may be an apparatus that uses a different cleaning technique altogether. For example, in one embodiment, a cleaning apparatus as shown and described in International Publication No. WO2016/180978, which is incorporated by reference herein. The systems 100 in FIGS. 34 and 36-39 do provide the advantage of cleaning the belt 105 of the conveyor 103 in addition to the target(s), reducing the need to stop the conveyor 103 for cleaning. Nevertheless, the embodiment of FIG. 40 can be used in combination with these systems 100 to provide additional cleaning and further reduce necessary downtime.

FIGS. 41-44 illustrate a system 120 using an apparatus 2 as described herein with a testing chamber 122 configured for use with the apparatus 2. In this embodiment, the testing chamber 122 has an opening 123 configured to receive a portion of the apparatus 2 such that the nozzle 14 directs the stream 7 into the testing chamber 122. The testing chamber 122 also includes a sliding tray 124 located below the opening 123 on which samples (not shown) can be placed, and the tray 124 can slide into the testing chamber 122 to place the samples in the path of the stream 7 of the apparatus. The apparatus 2 in FIGS. 41-44 is configured to use a choke 60 with a butterfly valve 64, although other types of choke mechanisms may be used in other embodiments, including any other choking mechanism described herein, or other choking mechanisms. In one embodiment, a plug-type choke may be used, and such a plug-type choke may be passed into the testing chamber 122 on the sliding tray 124 in one configuration. The system 120 also includes a receptacle 125 configured to catch the runoff liquid that exits the nozzle 14. The testing chamber 122 is particularly useful for using the apparatus 2 on samples that may be hazardous, e.g., biological, chemical, or nuclear, or for applications where the run-off is to be captured, e.g., for testing such as PCR analysis, polymerase chain reaction, etc.

The use of coupled modes facilitates the use of an apparatus 2 that includes a long, extended nozzle 14 and/or is directly connected to a pipe 84 or similar structure to clean the pipe. When such an extended nozzle 14 and/or a pipe 84 is connected to the body 4 and configured (along with the transducer 22) to conduct acoustic energy using coupled modes with the liquid, the surface waves of the acoustic energy can travel down the nozzle 14 and/or the pipe 84 to excite bubbles far down the stream. In the case of a pipe 84, the acoustic energy can be transmitted a significant distance down the length of the pipe 84, which may accomplish cleaning of the pipe 84. In such a configuration, the modes of the pipe 84, as well as the body 4 and/or the nozzle 14, must be coupled with that of the liquid, such that the mode of the pipe 84 matches the body 4 and/or the nozzle 14. In order to achieve this, the joints or connections between the nozzle 14 and the pipe 84 must be sufficiently secure to allow good acoustic transmission between the two (e.g. through soldering or welding cleanly, with the pipe 84 and nozzle 14 pressed to one another, or press-fitting the pipe 84 and the nozzle 14). If the pipe 84 and the nozzle 14 are separated by structures with different acoustic properties (such as gaskets) or structures that cause reflection of the coupled mode (such as air gaps), or if the joint contains significant changes to mass of area (such as flanges), then efficient transmission of coupled modes between the nozzle 14 and the pipe 84 can be impeded.

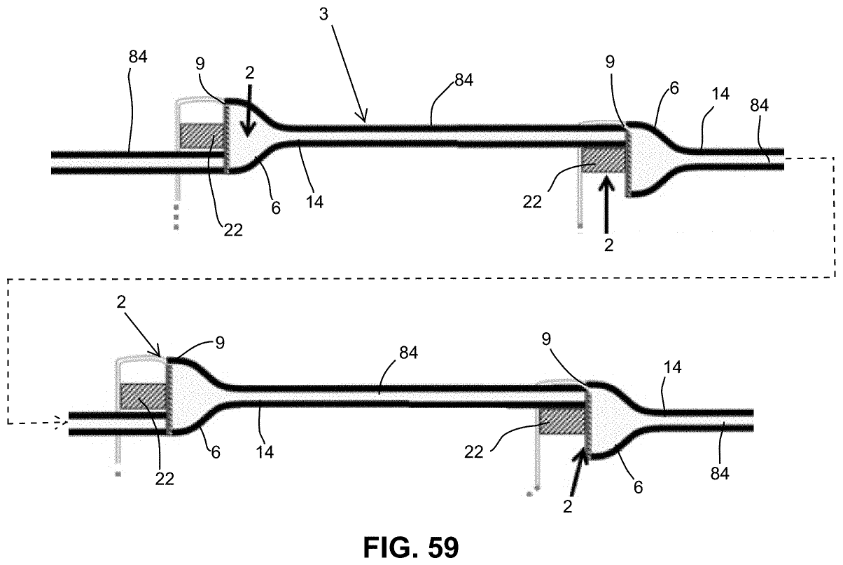

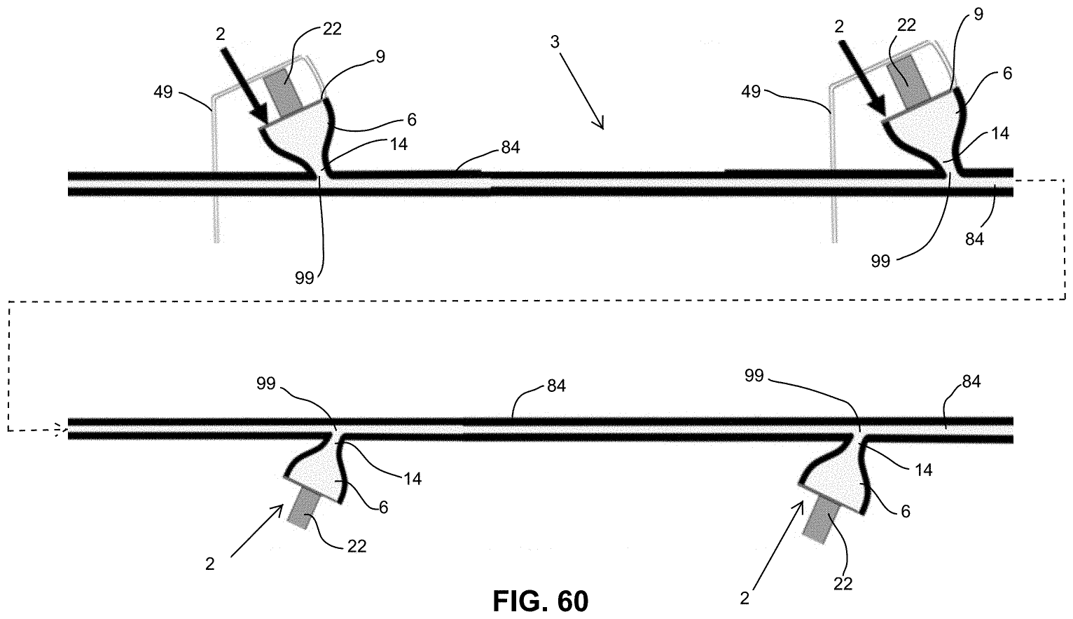

FIGS. 59-62 show various configurations of apparatuses 2 that are connected to pipes 84 to accomplish cleaning of the pipe 84. Such a configuration may be advantageous, for example, for cleaning beer lines in bars and pubs, pipework in drink and sugary beverage dispensers, and water pipes that might become contaminated (e.g. with Legionella), which need to be regularly cleaned to prevent the build-up of microbial biofilms and other contamination. Commonly, such cleaning is done with bleach or other chemicals, which leave an unpleasant taste and contamination when liquid product is next sent down the pipes. Liquid product is therefore sent down the pipes 84 after cleaning to flush the cleaning agent out of the pipework before the product is provided to customers, which results in wasted product. Similar procedures occur when liquid is sent through pipe work in the manufacturing, food and packaging industries. In the power industry (e.g. nuclear power), coolant pipes may become clogged by deposits over time, and are inaccessible to clean either from the inside or the outside. It is therefore advantageous to direct liquid flow into such a coolant pipe that cleans a length of the pipe from a remote access point where the acoustic energy can be introduced. This introduction might be done through ports, as shown in FIG. 60 and described below, or by permanently embedding the apparatuses 2 into pipework and only activating them when required, as shown in FIG. 59 and described below (although this structure could be used for FIG. 60 as well). The use of an apparatus 2 described herein, with appropriate mode coupling, permits such cleaning to be done without (or with minimal use of) cleaning chemicals, as the cleaning may be accomplished by use of ultrasonically-activated bubbles in a liquid. The liquid used in such embodiments may be or include one or more cleaning agents, if desired. In one embodiment, a system 3 may be provided with one or more apparatuses 2 as described herein built into pipework that includes one or more pipes (i.e., fluid conduits), such as water pipes, tap lines for transferring a liquid beverage (e.g., beer, soft drinks, etc.) from a container to a tap, internal conduits for a beverage dispensing machine, and other such pipework. While the pipes 84 in the embodiments of FIGS. 59-62 are illustrated as being straight, it is understood that pipes 84 with natural curvature or flexible pipes 84 may be used in some embodiments. If the pipe 84 is full of liquid (e.g., by filling from below or using sufficient pressure to exhaust the pipe 84) then the coupled modes in the nozzle 14 and the pipe 84 allow the cleaning action to extend along the pipe.

FIG. 59 illustrates one embodiment of a system 3 that includes a plurality of pipes 84 with a plurality of in-line apparatuses 2, such that the pipes 84 extend from the nozzle 14 of an upstream apparatus 2 to be placed in communication with the inlet 18 of a downstream apparatus 2. While this aspect is shown schematically in FIG. 59, each apparatus 2 in line in this embodiment has a manifold 40 as described herein, and each downstream apparatus 2 has its manifold inlet 42 connected to the pipe 84 extending from the nozzle 14 of the upstream apparatus 2. In another embodiment, the inlet 18 of each apparatus 2 may be configured differently (including potentially without a manifold 40), and the inlet(s) 18 of the downstream apparatus(es) 2 may be placed in communication with the pipe 84 extending from the upstream apparatus 2 in a different manner. It is understood that each pipe 84 may be provided in the form of an extended nozzle 14 in one embodiment, or may be connected to the nozzle 14 in another embodiment. Each apparatus 2 is configured to produce a stream of liquid from the inlet 18 out through the nozzle 14 and into the downstream pipe 84, which stream can be selectively provided with energized resonant bubbles for cleaning the pipe 84 (or other treatment) as described herein. Each apparatus 2 is also provided with a vent 9, and may also be provided with a choke (not shown) and/or a bubble generator (not shown) as described herein. It is noted that if the body 4 is pointing upwards or nearly upwards, a choke and/or exhaust may not be necessary, such as if the apparatus 2 is mounted on a vertical or near-vertical (e.g., within 50° of vertical) section of pipe. Transmission of the acoustic energy down the pipe 84 is facilitated by the use of coupled modes as described herein, in order to acoustically activate bubbles within the liquid down a length of the pipe 84. It is also understood that in the embodiment of FIG. 59, the liquid traveling through the pipes 84 passes through each apparatus 2 at all times, regardless of whether cleaning is being performed.

FIG. 60 illustrates another embodiment of a system 3 that includes one or more pipes 84 with a plurality of apparatuses 2 that have nozzles 14 in communication with the pipe(s) 84 to inject streams of liquid into the pipe(s) 84. In this embodiment, the inlets 18 of the apparatuses 2 are separately supplied with liquid that may not be sourced from the pipe(s) 84, such as by each apparatus 2 having an individual liquid supply conduit 20 (not shown in FIG. 60). As a result, the apparatuses 2 may be selectively activated to supply liquid to the pipe(s) 84 only when desired, such as during a cleaning cycle. The apparatuses 2 may also be configured to continuously supply liquid to the pipe(s) 84 if desired. In another embodiment, the system 3 of FIG. 60 may include bypass lines (which may be selectively opened and closed) to connect the inlets 18 of the apparatuses to the pipe(s) 84, so as to supply liquid from the pipe(s) 84 to the inlets 18. The apparatuses 2 in FIG. 60 are connected to the pipe(s) 84 by ports 99 positioned periodically at spaced locations along the length of the pipe(s) 84. Each port 99 is configured to permit the nozzle 14 of the apparatus 2 to inject liquid into the pipe 84, such as by a fitted or mating connection. The apparatuses 2 may be removed from the pipe(s) 84 when not in use or may remain connected to the pipe(s) 84 indefinitely. In another embodiment, the apparatuses 2 may be permanently connected to the pipe(s) 84 in the configurations illustrated. The two upstream apparatuses 2 in the system 3 of FIG. 60 have nozzles 14 that exit at least partially downward and are therefore provided with vents 9 as described herein, and may also be provided with a choke (not shown) as described herein. The two downstream apparatuses 2 in the system of FIG. 60 have nozzles 14 that exit at least partially upward, and vents and/or chokes may not be necessary in such a configuration. Transmission of the acoustic energy down the pipe 84 is facilitated by the use of coupled modes as described herein, in order to acoustically activate bubbles within the liquid down a length of the pipe 84.

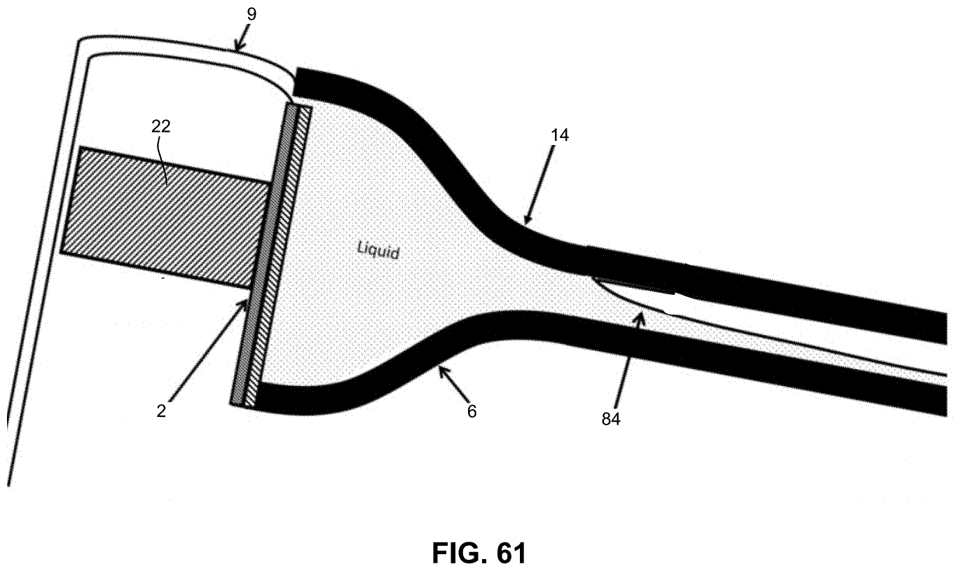

FIG. 61 illustrates an embodiment of a system 3 that includes an apparatus 2 that may be used in connection with a system 3 as in FIGS. 59-60 or another system 3 that includes one or more apparatuses 2 configured to supply liquid with acoustically-activated bubbles as described herein. In this embodiment, the apparatus 2 includes a rear wall 8, a transducer 22, a vent 9, and a manifold 40 as described herein, which are schematically illustrated. The apparatus 2 may also include a choke and/or a bubble generator as described herein. The nozzle 14 of the apparatus 2 in FIG. 61 is acoustically coupled with the pipe 84 in a manner that permits propagation of acoustic energy along the wall of the nozzle 14, and from the nozzle 14 directly into and through the wall of the pipe 84. In one embodiment, the nozzle 14 and the pipe 84 may be integrally formed together, such as being formed of a single piece of a single material. In another embodiment, the nozzle 14 and the pipe 84 may be formed of separate pieces that are connected in such a way as to permit transmission of acoustic energy from the nozzle 14 to the pipe 84, and are made of materials that permit coupled modes to be achieved between the liquid and the walls of the nozzle 14 and the pipe 84. The nozzle 14 and/or the pipe 84 may be provided with a fitting (not shown) that permits this acoustic coupling. In such a configuration, the use of mode coupling as described herein permits the acoustic energy to be transmitted down the pipe 84 to acoustically activate bubbles along at least a portion of the length of the pipe 84. This transmission of acoustic energy is even possible when the level of liquid in the pipe 84 is too thin for the acoustic energy to propagate effectively through the liquid, as shown in FIG. 61. Thus, in this configuration, high fluid pressure does not need to be maintained within the body 4 and the pipe 84. Cleaning can occur in this embodiment even when the pipe 84 is only partially full, with the understanding that such cleaning only occurs where the liquid touches the pipe wall.

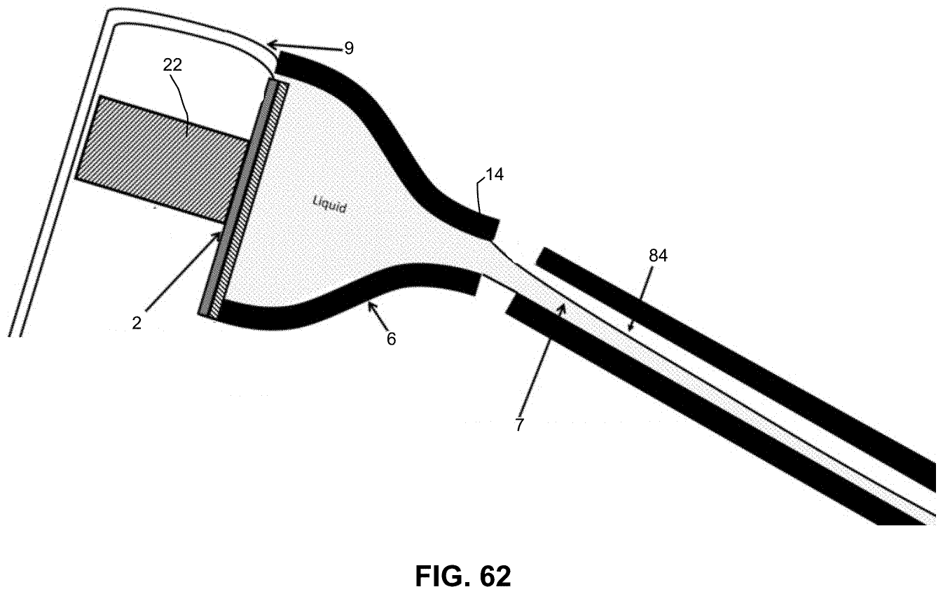

FIG. 62 illustrates another embodiment of a system 3 similar to the system 3 of FIG. 61, with the nozzle 14 not acoustically coupled to the pipe 84 via the solid wall material. In this configuration, water exiting the nozzle 14 travels down the pipe 84 in the same manner as shown in FIG. 62, but acoustic energy cannot be propagated directly from the nozzle 14 to the pipe 84. Mode coupling may still be used in the embodiment shown in FIG. 62, and the liquid stream alone must carry the acoustic energy into the pipe 84 to create coupled waves between the liquid and the pipe 84. Thus, in this embodiment, the characteristics of the stream must be sufficient to allow propagation of the acoustic energy from the nozzle 14 into the pipe 84, such as the thickness of the stream, the turbulence of the stream, the volume of bubbles in the stream, etc. It is understood that while FIG. 62 shows the nozzle 14 as being physically separate from the pipe 84, this embodiment may operate with the nozzle 14 physically connected to the pipe 84 in a non-acoustically coupled manner that does not permit transmission of acoustic energy directly from the nozzle 14 to the pipe 84 via the solid wall material. As in the embodiment of FIG. 61, cleaning can occur in this embodiment even when the pipe 84 is only partially full.

The apparatuses 2 shown in FIGS. 61-62 can be used to clean a pipe 84 with a larger diameter than the liquid stream from the apparatus 2, or when the outflow from the pipe 84 exceeds the inflow from the apparatus 2, or when a gas space builds up in the pipe 84. Despite the layer of liquid being too thin to allow for acoustic waves to travel solely in the liquid along the surface of the pipe 84 (if the cut-off frequency of the lowest mode is higher than the ultrasonic frequency if the film of water is considered to be a waveguide and coupled waves are ignored), cleaning can occur down at least a portion of the length of the pipe 84. This is enabled by the use of coupled modes as described herein.

In one embodiment, one or more apparatuses 2 are integrally connected within the pipework of the system 3, such that the apparatus(es) 2 are not disconnected from the system 3 during normal use. FIGS. 59-60 illustrate such embodiments. In such a system 3, the transducer is only activated during a cleaning (or other treatment) process, and not in normal use of delivering the liquid for consumption. In another embodiment, one or more apparatuses 2 may be connectable to and disconnectable from the pipework of the system 3, so that the apparatus(es) 2 deliver liquid to the pipes 84 only during cleaning or other treatment and can be removed during normal operation. The embodiments of FIGS. 61-62 may be configured to be permanently or removably connected to the system 3.

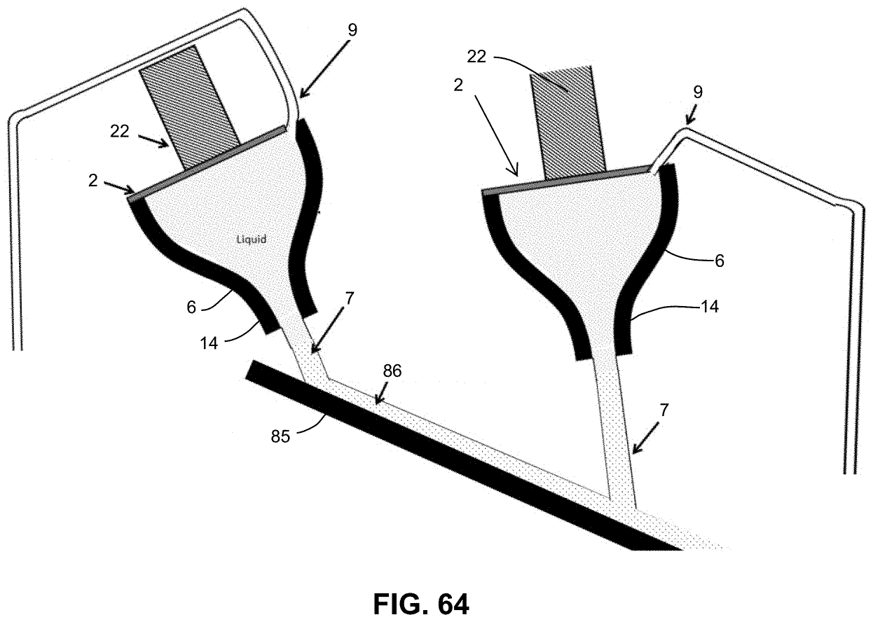

As described herein, a thin layer of liquid ordinarily cannot propagate acoustic energy effectively, but through the use of coupled modes between a thin layer of liquid and the solid surface next to it, acoustic energy can be propagated a significant distance to activate bubbles. For example, through the use of coupled modes, acoustic energy can be propagated 20-40 cm with refinement, or several meters with tuning and sufficient power, or tens of meters with reinforcement of the sound field (e.g., as described with respect to FIG. 64). In this way, an apparatus 2 as described herein can clean beyond the diameter of the stream, even when the layer is so thin that acoustic waves would not be able to travel through the liquid. Acoustic energy transferred into coupled modes and interface and surface waves can be used to extend the cleaning area. As discussed herein, propagation of acoustic waves in this manner can even be accomplished without the nozzle 14 touching the surface hosting the liquid film. This permits the use of an apparatus 2 as described herein for cleaning a wide variety of different target surfaces that are separate from the nozzle 14, such as the techniques for cleaning pipes 84 described above.

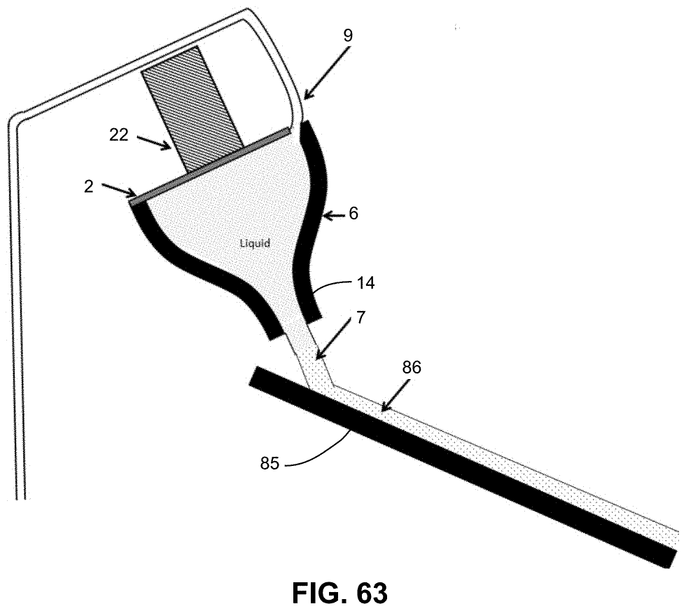

Another such example is cleaning or other treatment of a sheet material onto which the stream of water can be directed, such as a solar panel. FIGS. 63-64 illustrate example embodiments of using one or more apparatuses 2 as described herein to clean a target surface in the form of a solar panel 85, which has an inclined surface 86. In this embodiment, the water flows down the inclined surface 86 so that cleaning can occur both at the impact point of the stream and along the flow path of the liquid as it flows down the surface 86. If the acoustic energy and is configured (e.g., with the proper frequency) to excite coupled modes and interference waves, between the liquid and the surface 86 of the solar panel 85, a significant length of the flow path along the surface 86 can be cleaned as described herein. This permits a large surface area can be cleaned with relative speed and ease. One or more additional apparatuses 2, with one or more additional streams, may be used to reinforce the coupled modes and interface waves, in order to extend the length of the surface 86 within the flow path that is cleaned. FIG. 64 illustrates such an embodiment, where a second apparatus 2 has a nozzle 14 directed to produce a stream that intersects the flow path of the first stream on the surface 86. In this embodiment, both apparatuses 2 are configured to produce acoustic energy having the same properties (e.g., frequency) designed to create coupled modes with the surface 86 of the solar panel 85, which is propagated down the streams and onto the surface 86. These multiple streams reinforce the coupled modes within the flow path and increase the length of the flow path within which effective cleaning is accomplished. The streams could be delivered onto the surface 86 using a configuration such as shown in FIG. 35 (e.g., using nozzles 14 configured and arranged similarly to those shown in FIGS. 36-38), in which case exhaust tubes and/or chokes may not be required.

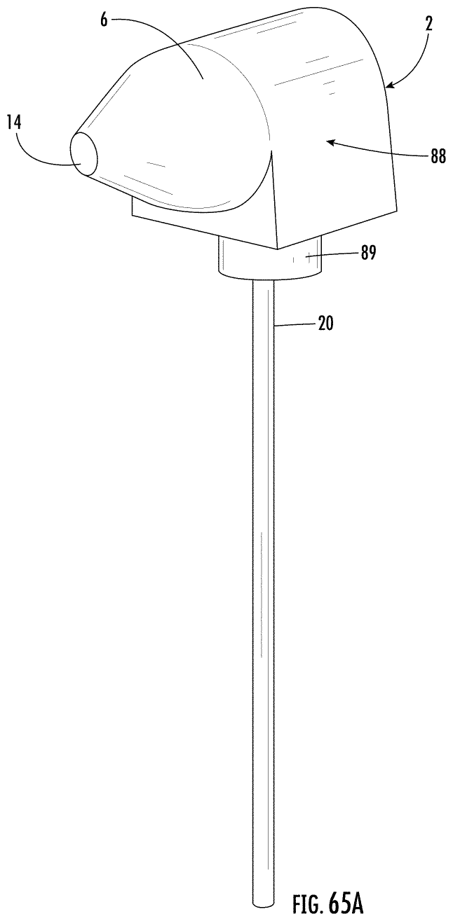

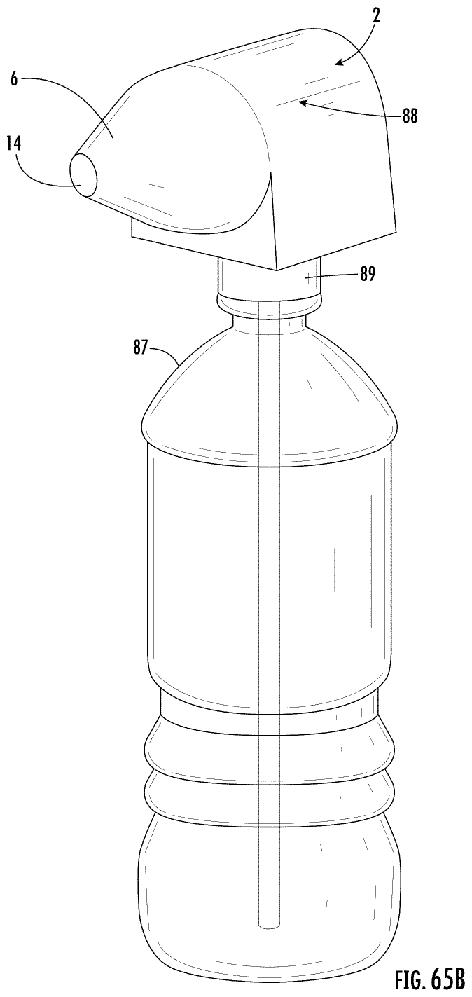

FIGS. 65A-B illustrate another embodiment of an apparatus 2 configured for use with a separate and/or removable container 87 illustrated in the form of a replaceable bottle, such as a disposable plastic bottle or a reusable bottle (e.g., including water or saline solution). The container 87 acts as the supply of liquid in this embodiment. It is understood that, while this embodiment is illustrated as being used with a bottle, other containers 87 may be used in place of a bottle, such as a compressible bag with a fitting for connection to the apparatus 2. The apparatus 2 includes a casing 88 that encloses at least some of the components of the apparatus 2, and which has a connector 89 that is configured for connection to the container 87. The connector 89 in FIGS. 65A-B is illustrated as a fitting that directly connects to the container 87, but the connector 89 may include intermediate structures in other embodiments, such as a flexible tube or other conduit extending from the container 87 to the chamber.

In the embodiment of FIGS. 65A-B, the apparatus 2 may include various components described herein, including at least a body 4 that defines a chamber (not shown), a nozzle 14, and a transducer (not shown). The apparatus 2 may include further components described herein, such as a vent, a manifold, a bubble generator, and/or a choke (none of which are shown), as well as other components, such as outgassers, filters, etc., to remove bubbles from the liquid. The liquid supply conduit 20 in FIGS. 65A-B extends downward within the connector 89 to extend into the container 87, which allows the apparatus 2 to draw liquid from the container 87 into the chamber, where the liquid is then forced out through the nozzle 14. In other embodiments, the liquid supply conduit 20 may be differently configured, and may not need to extend into the container 87 in some embodiments, depending on the configuration of the container 87 and the mechanism by which liquid is forced out of the container 87. The connector 89 is configured for connection to an opening of the container 87, such as by a threaded connection as shown in FIG. 65B.

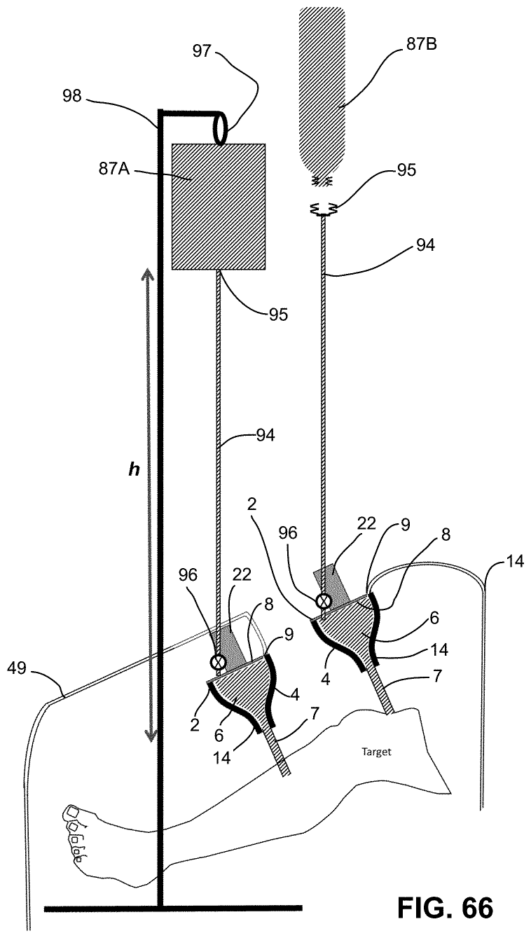

FIG. 66 illustrates additional embodiments of an apparatus 2 configured for use with a separate and/or removable container 87, through connection to a connection tube 94 or other conduit in fluid communication with the chamber 6 and the container 87. In this configuration, the connection tube 94 may feed directly into the inlet 18 for the chamber 6 of the apparatus 2, or may be connected to another component that feeds the liquid to the inlet 18, such as a manifold 40. The connection tube 94 may have a specialized connection member 95 at the end of the connection tube 94 distal from the apparatus 2, for connection to one or more container types. For example, the left hand apparatus 2 in FIG. 66 has a connection tube 94 with a connection member 95 configured for connection to a container 87A in the form of a bag or box that contains the liquid to be applied to the target. As another example, the right hand apparatus 2 in FIG. 66 uses has a connection tube 94 with a connection member 95 configured for connection to a container 87B in the form of a water bottle or other bottle for containing the liquid to be applied to the target. The connection tube 94 may also include a valve 96 positioned between the connection member 95 and the liquid inlet 18 for controlling flow of the liquid into the chamber 6. The valve 96 may be configured for manual or automatic activation, and may be mechanical or electronic. In the embodiments of FIG. 66, the connection tube 94 is configured to function as a siphon or gravity feed, by lifting the container 87A, 87B above the level of the chamber 6. As described herein, the connection tube 94 may be configured for forcing the liquid from the container 87A, 87B, such as by use of a pump, pressurization, mechanical compression, etc. The connection tube 94 may be flexible in one embodiment.

The connection member 95 may have a structure that is complementary with the structure of the container 87A, 87B. For example, the connection member 95 configured for connection to the container 87A in the form of a box or bag may connect to a complementary connection member (not shown) on the container 87A, which may have a structure configured for mating, interlocking, and/or sealing with the connection member 95. If the container 87A is specially configured for connection to the connection member 95, the container 87A may also be provided with an air inlet (not shown), such as a one-way valve that allows air to enter the container 87A to replace the liquid exiting to the chamber 6. As another example, the connection member 95 configured for connection to the container 87A in the form of a box or bag may have a lancing member (not shown) for piercing the container 87A. As a further example, the connection member 95 configured for connection to the container 87B in the form of a bottle may be in the form of a threaded connector, as illustrated in FIG. 66. For clarity, the threaded connection member 95 at the top of the connection tube 94 in the right hand apparatus 2 is shown disconnected from the container 87B, but in operation it would be connected to the container 87B to allow the liquid to flow from the container 87B into the connection tube 94 without spillage where it meets the connection member 95. This connection of the connection member 95 onto the container 87B is made before the container 87B is inverted such that the open end of the container 87B is lower than its base. The apparatus 2 in FIG. 66 may also be provided with a stand 98 configured to hold the container 87A, 87B in an elevated position during use, to facilitate flow of liquid from the container 87A, 87B to the chamber 6 and through the nozzle 14 by siphon or gravity effect. The stand 98 is illustrated in FIG. 66 as being configured for suspending the container 87A in the form of a bag or box, e.g., with a support member 97 in the form of one or more hooks, strings, wires, screws, etc. Other embodiments of a stand 98 may be configured for use with one or more specific types of containers 87. For example, a stand 98 may be provided with a support member 97 for holding the container 87B in an inverted position, such as one or more hooks or screws, one or more wires or strings, a cage or frame, etc. The stand 98 may be collapsible in one embodiment for ease of transport.

The height of the stand 98 is sufficient to supply the hydrostatic head that enables the liquid to be supplied to the apparatus 2 without use of a pump, which provides multiple benefits. As one example, this removes the need to supply electrical power for a pump, enabling use in a wider variety of situations. As another example, the lack of a pump (and a power supply therefor) reduces the weight and cost of the apparatus 2 and the associated components. As a further example, a pump can introduce bubbles into the liquid, which may be avoided if a pump is not used. The apparatus 2 of FIG. 66 functions best when the flow rate is about 0.25-0.5 L/min, and if the liquid flow speed from the nozzle 14 used is sufficiently fast to reduce instabilities on the wall of the stream and/or stream breakup, a 500 mL container 87 of liquid would last for 1-2 minutes, and a 250 mL container 87 would last for 3-4 minutes. To achieve this condition, a nozzle inner diameter of about 2 mm and an acoustic frequency about 1 MHz may be used. If h is the height difference between the top of the connection tube 94 and the exit from the nozzle 14, then (ignoring losses during the flow by turbulence, circulation, friction, etc.) equating potential energy of liquid at top of tube (where it assumed to be close to stationary) with the kinetic energy when it leaves where g is the acceleration due to gravity, and the liquid density is given by p. In practice, a slightly greater height difference h will be required to overcome the losses (perhaps 10%-50% greater). A pump could be added to enhance flow speed, but carries the disadvantages discussed herein. The height of the stand 98 should be sufficient to create a sufficient height h to provide the desired liquid speed through the nozzle 14, as shown in FIG. 66.

It is noted that with the conditions described above (e.g., a nozzle diameter of about 2 mm and an acoustic frequency of about 1 MHz), bubbles larger than about 6 microns in diameter are considered to be unwanted bubbles that are not acoustically activated, but instead simply attenuate the sound field in the stream 7 and chamber 6 through scattering. Therefore, measures to reduce bubbles in the liquid flow may be advantageous, such as not using a pump, letting the container 87 settle before use, taking the liquid from the bottom of the container 87 away from the buoyant bubbles, and possibly degassing the liquid supply (e.g., by heating the container 87, keeping the container 87 under partial vacuum, ultrasonic degassing, etc.) followed by sealing from the atmosphere (e.g., while the liquid cools). This degassed liquid may be sealed from the atmosphere in a container that contains no or minimal gas space to avoid shrinkage of the sealed container under atmospheric pressure as the gas space cools, and to avoid gas re-absorbing into the liquid.

The apparatus 2 in FIG. 66 may have an exhaust 49 with a tube that is sufficiently narrow so as to prevent air entering from the water exit point (i.e., the end of the exhaust 49), and by placing the water exit point sufficiently far beneath the nozzle, a siphon can be set up for vent 9 to remove unwanted gas/bubbles from the chamber 6. Such a siphon effect can be assisted by use of a choke 60 when the chamber 6 first fills with liquid as discussed herein.

One example of a method of use of the apparatus 2 as shown in FIG. 66 (using a bottle as the container 87B) would include attaching the container 87B to a fixing point to ensure that the distance h above the tip of the nozzle 14 is sufficient to achieve fast enough flow from nozzle 14, but not so great as to empty the container 87B too quickly, as described herein. Such a fixing point may be provided by a stand 98, a hand of a colleague, a hook or other attachment on a wall of an ambulance or building, or other elevated fixing point. The container 87B should be positioned upright before use, so the cap can be removed and the connection member 95 attached without spillage. Any additives (e.g., saline salts, biocide, antiseptic, drugs, etc.) to be added to the liquid should be added prior to connecting the connection member 95. While this container 87B configuration is suitable for using with a standard drinking water bottle to provide a treatment-of-opportunity to a surface (e.g. the wound of an injured mountain climber), the container 87B may also come pre-prepared (e.g. sterilized, and possibly with antiseptic or biocide or antimicrobial added) and sealed, so that water only issues from the container 87B when the seal is pierced. The connection member 95 may include piercing implement that pieces the seal on the container 87B when it is connected to the container 87B, e.g., as the connection member 95 is screwed on. Such an integrated piercing implement may be used with other containers 87 and connection members 95 as well. After the connection member 95 is attached, the user then ensures that the nozzle 14 is ready and in place to deploy the liquid, with the exhaust 49 in position, the choke 60 closed, and any other conditions for beginning operation are satisfied. At that point, the container 87B is raised up to the fixing point (and attached, if applicable), in an inverted position so the base of the container 87B is at the top. The base (now the top) of the container 87B is then pierced to allow air flow into the container 87B, and the base of the container 87B should be cleaned prior to piercing to avoid contamination. The valve 96 for the connection tube 94 can then be opened to begin the flow of liquid into the body 4 and out through the nozzle 14. The target can then be treated using liquid, bubbles, and acoustic energy as described herein.

In one embodiment, an apparatus 2 as shown in FIG. 66 may be deployed by use of a drone or other aircraft capable of hovering flight. A small (e.g., 500 mL) liquid container 87 could be carried by the drone along with the apparatus 2, and the drone may be deployed when a call or other alert is received indicating that treatment is needed. The drone may also act as an elevated fixing point for the container 87 (e.g., by hovering) and could supply electrical power for the transducer 22 and controller 19.

An apparatus 2 such as shown in FIG. 65A-66 may be provided as part of a kit that includes the apparatus 2 and other components for use with the apparatus 2. For example, the apparatus 2 may be provided with one or more packets of additives (e.g., chemicals) that can be added to the liquid in the container 87 to provide desired properties, such as for cleaning, treatment, etc., as described herein. One such additive may be a powder to convert water into saline solution, e.g., to prevent pain and/or adverse effects (such as osmotic stress and osmotic shock) from the unwanted effects of an induced osmotic gradient on application in deep wounds, anatomical pockets, etc. As another example, an apparatus 2 may be provided with a stand 98 and/or one or more containers 87 filled with liquid. As a further example, an apparatus 2 may be provided with a piercing member (not shown) for piercing the container 87 to allow air to enter during use, such as a blade or spike. It is understood that such a kit may include any combination of these components and/or other components as well.

Any of the apparatuses 2 in FIGS. 65A-66 may be configured for forcing liquid from the container 87 through the supply conduit 20 to the chamber and out through the nozzle 14. In one embodiment, the apparatus 2 may have an internal pumping mechanism (not shown) to draw liquid from the bottle and eject the liquid from the nozzle 14, and various pumping mechanisms may be used. A peristaltic pump may provide the benefit of reducing cavitation and/or bubble entrainment as compared to some other pumping mechanisms, such as an impeller-based pump. In another embodiment, the container 87 may be configured to be pressurized, such as by use of a pre-pressurized container 87 or by use of a pressurization mechanism on the apparatus 2 that functions to pressurize the container 87 when the apparatus 2 is connected to the container 87. The container 87 could also be pressurized by application of external force, such as manually squeezing the container 87, placing weighted objects (e.g., stones) on the container 87, etc. Pressurization by application of external force may function more effectively if a bag or similar structure is used in place of a bottle. In a further embodiment, a siphon or gravity feed mechanism may be used to create liquid flow from the container 87 to the apparatus 2, such as by raising the container 87 above the liquid level in the chamber, such as in FIG. 66. This embodiment may benefit from the connector 89 including a flexible conduit for establishing this siphon.

In one embodiment, any of the apparatuses 2 in FIGS. 65A-66 or other embodiments for use with a separate and/or removable container 87 may include a nozzle 14 with a relatively narrow diameter, e.g., less than 5 mm in one embodiment, or 1-3 mm in another embodiment, or 2 mm in a further embodiment. The use of a narrower diameter nozzle 14 allows the required liquid flow speed from the nozzle 14 (e.g., to suppress instabilities in the stream) to be achieved with a relatively lower volume flow rate, as described herein. Lower volume flow rates are important in a portable apparatus that uses a separate and/or removable container 87, because the total volume of liquid available for use may be relatively low. As described herein, the use of a smaller nozzle diameter may require the use of higher frequency acoustic energy, based on the cut-off frequency. The use of higher frequencies, in turn, necessitates the use of smaller bubbles to achieve pulsation resonance. In the event that a larger water supply is available, a larger nozzle 14 may be used, such as a 10 mm diameter nozzle 14. Outgassing and/or pre-filtering may therefore be beneficial in this embodiment. Higher hydrostatic pressure may be necessary if a filter is placed between the container 87 and the apparatus 2, which would increase the required distance h between the nozzle 14 and the container 87. Gas removal may also be accomplished by letting the liquid sit (e.g., after elevating the container 87 to the height h) for 30-90 minutes to allow bubbles to rise out, before starting the flow.

Any of the apparatuses 2 of FIGS. 65A-66 may further include an integrated power source (not shown), e.g., an internal battery, to make the apparatus 2 completely portable and usable in any location. The apparatus 2 may also be powered by an external power source, such as a main power or various dry or wet cell batteries, including a vehicle battery, a marine battery, a rechargeable battery, a solar-powered rechargeable battery, or any other various power cells. Connection to a vehicle battery may be made through a cigarette lighter socket, USB, or other connection on a vehicle, or through direct connection to the battery, e.g., by cables with appropriate connections (e.g., alligator clips). Connection to a marine battery on a watercraft may be similarly made. Such a portable apparatus 2 may be used by rescue workers in hazardous or remote areas, such as mountain rescues, disaster areas, battlefields, etc., by connecting the apparatus 2 to a container 87 filled with an appropriate liquid. The liquid could be transported with the user or obtained on-site (e.g., filtered stream water). This permits the cleaning of wounds in situations where adequate cleaning may not be possible otherwise, without heat, chemicals (e.g., bleach), or other agents that may cause damage to the tissue.

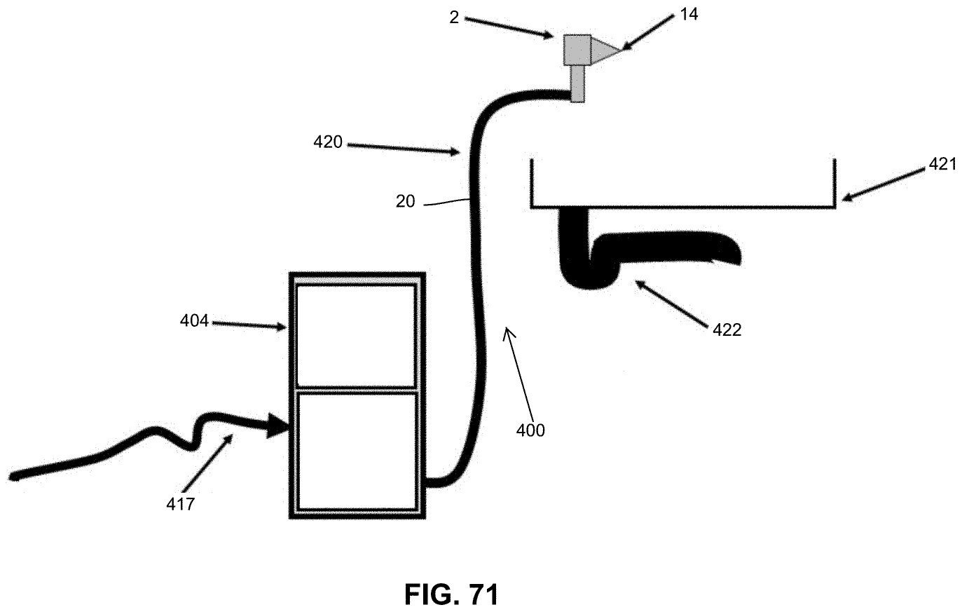

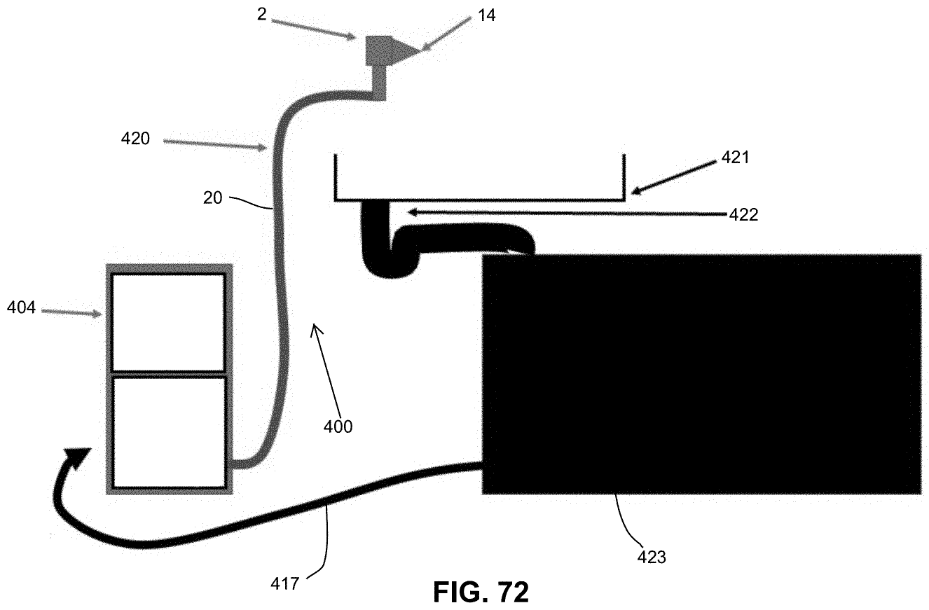

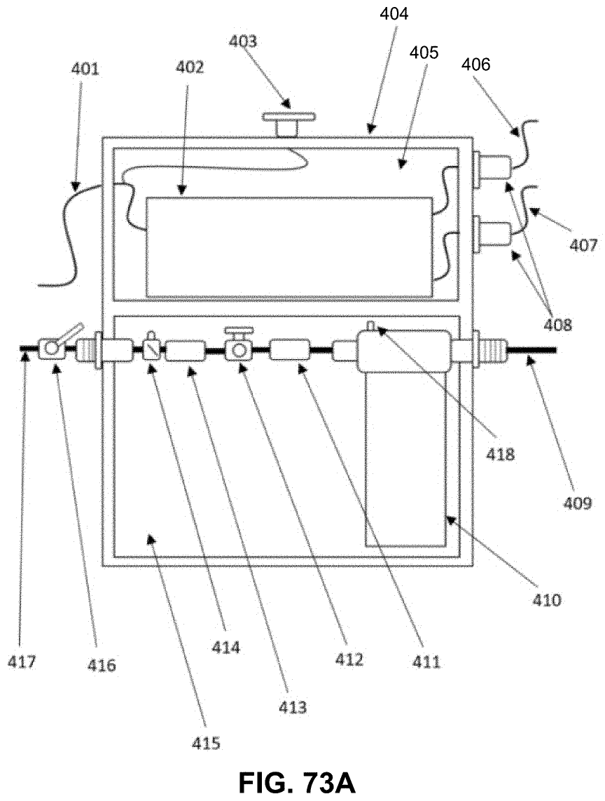

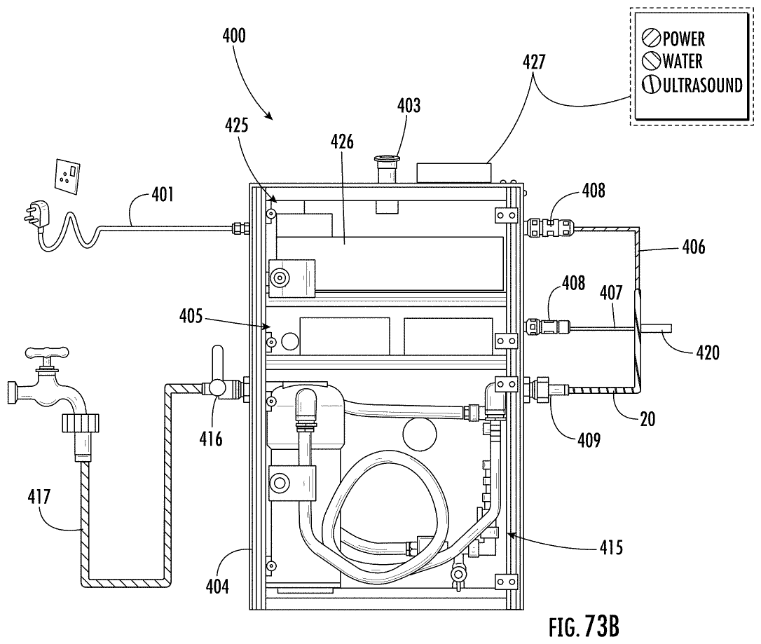

The apparatus 2 as described herein can be used with various different configurations of a liquid supply 417, shown schematically in FIGS. 71-73. For example, an apparatus 2 configured for use with water as the liquid may include a liquid supply 417 in the form of a main water supply (water main), a pumped water supply, a gravity feed from a header tank or bag, or a container 87 as shown in FIGS. 65-66 and discussed herein, among other configurations. The liquid supply used should be capable of generating the desired flow speed and volume flow of the liquid. However, some liquid supply apparatuses may introduce unwanted bubbles into the liquid, such as a main water supply or a pump. For example, maintenance on a main water supply or a fractured leak can entrain air into the liquid. As another example, certain types of pumps (particularly impeller-type pumps) can introduce bubbles into the liquid, such as through cavitation. Consequently, removal of these unwanted bubbles by an outgasser (such as a microporous filter) is desirable. In one embodiment, a control cabinet 404 may be provided for use with an apparatus 2 as described herein, which may include numerous components for operation of the apparatus 2, including electronic components (e.g., the controller 19 or components thereof), an outgasser, and other devices. FIGS. 71-72 illustrate embodiments of the apparatus 2 that use a control cabinet 404 as described herein, located between the liquid supply 417 and the liquid supply conduit 20. Such a control cabinet 404 may be designed to be portable, either by carrying or by provision of powered or non-powered wheels, casters, rollers, tracks, etc. In one embodiment, the control cabinet 404 may be approximately 20 cm×20 cm×30 cm in size, providing a portable configuration. The control cabinet 404 is illustrated schematically in FIGS. 71-72, where the control cabinet 404 is divided into multiple compartments that include a system control compartment 405 and a plumbing compartment 415. The system control compartment 405 may include components such as power amplifier, control electronics, and/or diagnostic electronics, while the plumbing compartment 415 may include components such as water outgassing and filtration, valves (e.g., solenoid-type), and plumbing. In the embodiments of FIGS. 71-72, one or more conduits (including liquid and/or electrical conduits) may extend between the control cabinet 404 and the apparatus 2 for placing the components of the control cabinet 404 in fluid and/or electronic communication with the apparatus 2. These conduits are consolidated within an umbilical 420 in FIGS. 71-72 extending from the control cabinet 404 to the apparatus, which includes multiple conduits held together, e.g., by one or more sheaths or bindings. This umbilical 420 may, in one embodiment, include at least the liquid supply conduit 20 and at least one electronic conduit, such as one or more electronic communication cables and/or a power cord, and may also include an exhaust conduit as well. FIGS. 73A-B, discussed in greater detail below, illustrate embodiments of such a control cabinet 404 in greater detail.

FIG. 71 illustrates an embodiment of a system 400 including an apparatus 2 as described herein with a control cabinet 404. In the embodiment of FIG. 71, the liquid supply 417 is in the form of a main water supply, which is connected to the cabinet 400, which then supplies the water to the apparatus 2 through the liquid supply conduit. It is understood that the system 400 in FIG. 71 may be used with a different type of liquid supply, including various pressurized liquid supply configurations. The apparatus 2 is illustrated for use with a sink or basin 421 with a drain 422 that removes the water after use in cleaning or treatment.

FIG. 72 illustrates another embodiment of a system 400 including an apparatus 2 as described herein with a control cabinet 404, where the liquid supply 417 is provided by recirculation from previously used liquid. In the embodiment of FIG. 72, the drain 422 from the basin 421 extends to a liquid recirculation system 423, which supplies the liquid to the cabinet 400, which then supplies the liquid to the apparatus 2 through the liquid supply conduit. The recirculation system 423 may include equipment such as pumps for moving the liquid, filters and other devices for cleaning the liquid, and/or a temperature control unit for cooling or heating as necessary to maintain a desired temperature of the liquid. The embodiment of FIG. 72 can be used to reduce or avoid wasting liquid, which is particularly advantageous in situations where access to the liquid (e.g., water) is difficult, or where there is some constraint on the supply of liquid.

In the embodiments of FIGS. 71 and 72, the target or targets may be placed within or over the basin 421 so that water used in cleaning or treating the target runs off into the basin 421 and into the drain 422. The basin 421 may be provided with specialized equipment, such as a holder for holding one or more targets within or over the basin 421.

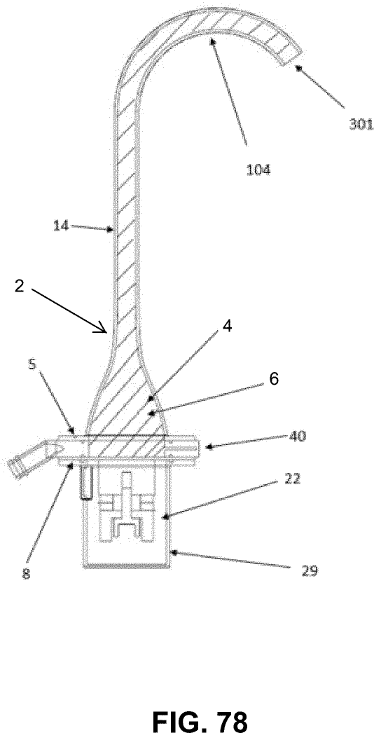

The systems 400 in FIGS. 71 and 72 may be provided as part of an integrated sink unit that includes some or all of the apparatus 2, the basin 421, the drain 422, the recirculation system 423 (if present), and the control cabinet 404. The apparatus 2 may be provided as part of such an integrated system 400, or may be provided separately, and different apparatuses 2 may be interchangeable with the system 400. The control cabinet 404 may be located within a sink cabinet, or the components of the control cabinet 404 may be built into a larger sink cabinet, such that the sink cabinet functions as the control cabinet 404. The apparatus may be stored in the control cabinet 404 and/or the sink cabinet when not in use, and a designated holder or compartment may be provided for storage of the apparatus 2. The sink cabinet in this embodiment may be provided as a fixture or as a self-contained portable system, i.e., including powered or non-powered wheels, casters, rollers, tracks, etc. The system 400 may be integrated into a single cabinet with the water to clean/treat coming from either a handheld or mounted apparatus 2 as shown in FIG. 72, or from a faucet 301 of the type described herein (e.g., FIGS. 78-86), could be used to provide a mobile cleaning station, especially if supplemented with soap, normal taps, vanity accessories such as mirrors, paper towels, and hand dryers etc., to provide support. Such a cabinet may also include any or all of a sink/basin 421, a drain 422, a recirculation system 423, heater/cooler units, filters, electronics, and an apparatus 2 (which may be configured as shown in FIGS. 71-72 or as a faucet 301 as described with respect to other embodiments herein such as FIGS. 78-86). The cabinet may be mounted on feet, wheels, casters, etc., to give it portability. A cabinet unit of this type may be rolled around a garage or workshop, or supplied to provide initial cover for field hospitals, mobile clinics, field research stations, wards, concerts and festivals (e.g. to supply extra hygiene). If main water and main wastewater and main electricity supplies are available, the system 400 could be connected to these, in which case the recirculation system 423 may be omitted or bypassed. If any or all of these main facilities (water, wastewater, power) are not present, the cabinet of the system 400 may be provided with a portable supply, such as a refillable water supply (e.g., from replaceable water bottle, by filling a tank in the cabinet using a hose, etc.), a wastewater storage unit, and batteries (e.g., with solar, charging, vehicle or marine battery power, or run from a vehicle or portable generator). The system 400 may also be configured for operating in multiple modes, including using recirculating water, or using water from a main supply or a supply stored in the cabinet with a drain 422 connected to a wastewater main or a storage container.

In one embodiment, separate water lines, one or more for cooled water and one or more for heated water (potentially exploiting a heat pump within the cabinet between the separate reservoirs for heated and cooled water) could be included in the recirculation system 423. Such a cabinet may also include a sink/basin 421, a drain 422 and/or a faucet 301 as described with respect to other embodiments herein. In one advantageous configuration, a cold water faucet with the acoustic energy capability of the apparatus 2 described herein (e.g., as in FIG. 78) is provided along with a hot water faucet with no transducer 22 or acoustic energy capability for normal cleaning, e.g., with soap, detergent, or degreaser.

FIG. 73A illustrates one embodiment of a control cabinet 404 that is usable with the embodiments of FIGS. 71-72 and other embodiments herein. The cabinet 404 in FIG. 73A is divided into two compartments: a system control compartment 405 and a plumbing compartment 415. These compartments 405, 415 may contain various components as described herein. The control compartment 405 in FIG. 73A contains a power amplifier and electrical control box 402 that may be powered from a main power supply 401, such as by a power cord connected to an outlet, and delivers power to the apparatus 2 via a high voltage power cable 406 and control signals via a low voltage communication cable 407. These cables 406, 407 may be detachable to aid portability using quick release connectors 408. An emergency stop 403 may be present to increase operator safety. The control compartment 405 may be provided with sealing that is resistant to water or other liquids, to protect from liquid ingress from the plumbing compartment 415 and the outside environment.

The plumbing compartment 415 generally includes components to control the liquid flow to the apparatus 2 and the condition of the liquid that reaches the apparatus 2. The liquid may enter the system via the liquid supply 417, which is a main water supply in FIG. 73A but may be a different source in another embodiment, such as another pressurized liquid source. A mechanical flow shut off valve 416 may be positioned at the connection of the liquid supply 417 to the control cabinet 404 to enable the user to shut of the flow to the cabinet 404. When the mechanical valve 416 is open, the liquid may flow through a solenoid valve 414 that controls the flow of liquid through the cabinet 404 and to the apparatus 2. The cabinet 404 may also contain a temperature sensor 413 and a pressure regulator 412, through which the liquid flows prior to entering the liquid supply conduit 20 to the apparatus 2. The cabinet 404 may further include a flow control valve 411 that could be fixed or user-controlled depending on the application. The cabinet 404 may additionally include a filter 410 (e.g., a microporous filter) or other outgassing device, to remove unwanted bubbles from the water, which filter 410 may have a pressure release valve 418 to remove any excess air upon installation. The liquid exits the cabinet 404 through a terminal 409 that is connected to the liquid supply conduit 20 and thereby travels to the apparatus. The liquid supply conduit 20, the power cable 406 and the communication cable 407 may be consolidated into a single umbilical 420 that connects the cabinet 404 to the apparatus 2, which is not illustrated in FIG. 73A. The walls of the control cabinet 404 may be configured for reducing electromagnetic interference (such as by being made of metal), either for protection of the components of the control cabinet 404 against leakage from other devices, or for protection against leakage from the control cabinet 404 affecting other devices or exceeding recommended levels. Similar protection may be provided in the umbilical 420 or the individual cables 406, 407, in the apparatus 2 (e.g., in the handle 56), and/or in any electrical connections between the same.

FIG. 73B illustrates another embodiment of a control cabinet 404 that is usable with the embodiments of FIGS. 71-72 and other embodiments herein. The cabinet 404 in FIG. 73B is divided into three compartments: a system control compartment 405, a plumbing compartment 415, and a power amplifier compartment 425. These compartments 405, 415, 425 may contain various components as described herein. The control compartment 405 in FIG. 73B contains an electrical control box 402 and associated components that may be powered from a main power supply 401, such as by a power cord connected to an outlet, and delivers control signals via a low voltage communication cable 407. The power amplifier compartment 425 contains a power amplifier 426 and associated components connected to the main power supply 401, which delivers power to the apparatus 2 via a high voltage power cable 406. These cables 406, 407 may be detachable to aid portability using quick release connectors 408. An emergency stop 403 may be present to increase operator safety. The control compartment 405 and the power amplifier compartment 425 may be provided with sealing that is resistant to water or other liquids, to protect from liquid ingress from the plumbing compartment 415 and the outside environment. The control cabinet 404 may further include an LED panel 427, which is positioned on the top surface in FIG. 73B, with detail shown in the inset. The control cabinet 404 of FIG. 73B and/or the electrical components and connections thereof may be shielded against electromagnetic interference as similarly described above with respect to FIG. 73A.

The plumbing compartment 415 generally includes components to control the liquid flow to the apparatus 2 and the condition of the liquid that reaches the apparatus 2. The liquid may enter the system via the liquid supply 417, which is a main water supply in FIG. 73B but may be a different source in another embodiment, such as another pressurized liquid source. A mechanical flow shut off valve 416 may be positioned at the connection of the liquid supply 417 to the control cabinet 404 to enable the user to shut of the flow to the cabinet 404. When the mechanical valve 416 is open, the liquid may flow through the control cabinet and to the apparatus 2, and the plumbing compartment 415 may include additional components for controlling, conditioning, and/or monitoring the water flow, such as a filter (e.g., a microporous filter) or another outgassing device. The liquid exits the cabinet 404 through a terminal 409 that is connected to the liquid supply conduit 20 and thereby travels to the apparatus. The liquid supply conduit 20, the power cable 406 and the communication cable 407 may be consolidated into a single umbilical 420 that connects the cabinet 404 to the apparatus 2, which is not illustrated in FIG. 73B. The control cabinet 404 in FIG. 73B may include any of the additional features shown in FIG. 73A and/or described herein, and the control cabinet 404 in FIG. 73A may include any of the additional features shown in FIG. 73B and/or described herein.

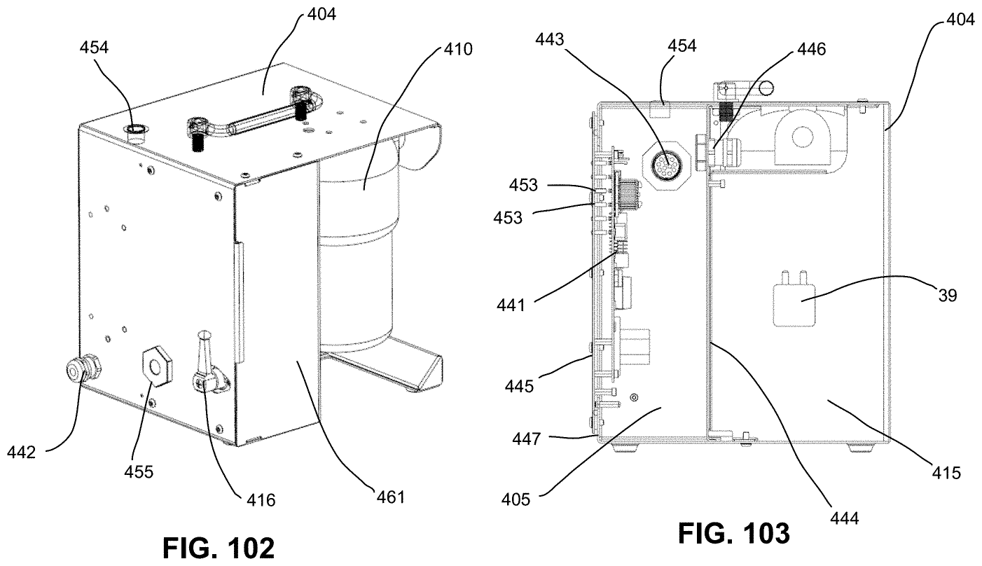

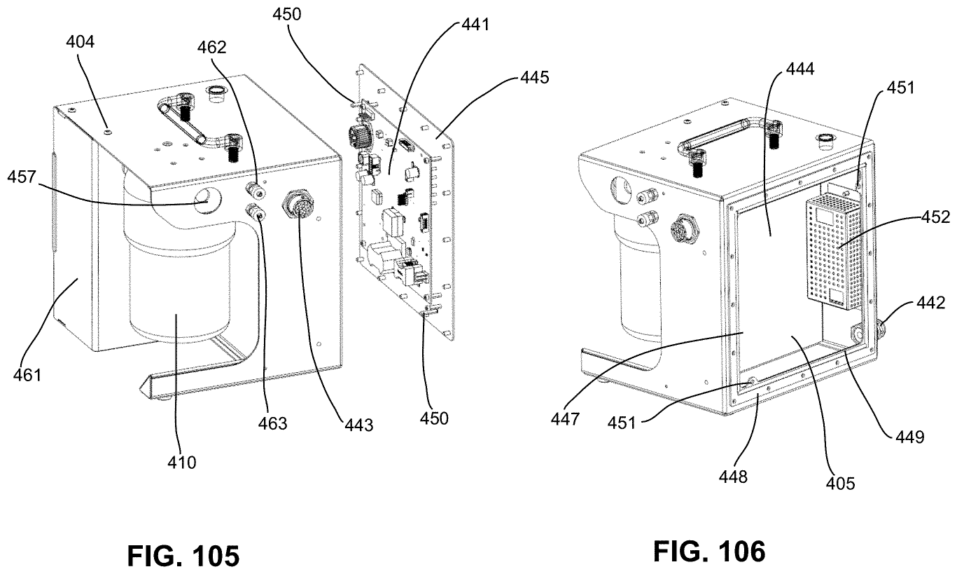

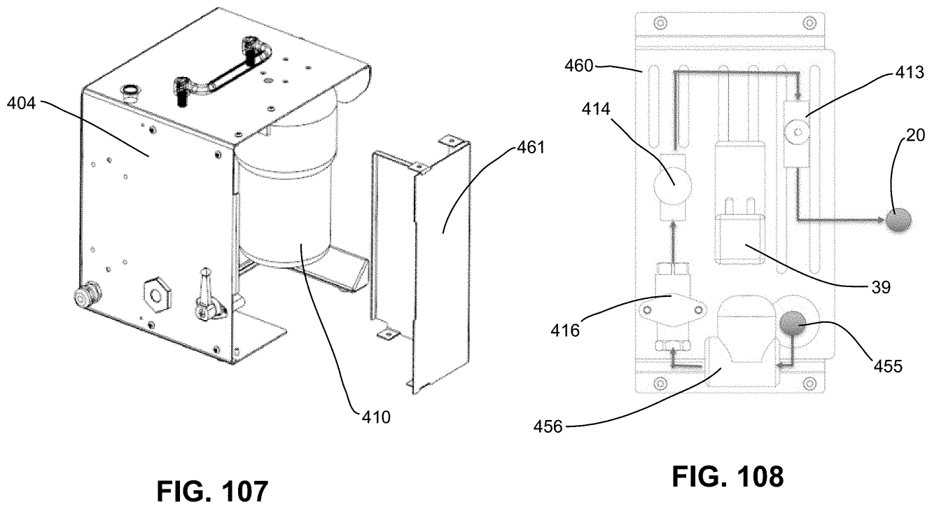

FIGS. 102-108 illustrate another embodiment of a control cabinet 404 that is usable with the apparatus 2 of FIGS. 89-101 as shown in FIG. 88, but may also be configured for use with other embodiments of apparatuses 2 as described herein. In the embodiment of FIGS. 88 and 102-108, the liquid supply 417 is in the form of a main water supply, which is connected to the cabinet 400, which then supplies the water to the apparatus 2 through the liquid supply conduit. It is understood that the system 400 and/or the control cabinet 404 in this embodiment may be used with a different type of liquid supply, including various pressurized liquid supply configurations. The cabinet 404 in FIG. 102-108 is divided into two compartments: a system control compartment 405 and a plumbing compartment 415. These compartments 405, 415 may contain various components as described herein. It is understood that certain common components, such as water and electrical conduits and connections (both internal and external to the control cabinet 404) may be illustrated only schematically, or not at all, in FIGS. 102-108. It is also understood that the control cabinet 404 may contain additional or alternate components as described herein, including any components described herein with respect to other embodiments, such as those shown in FIGS. 73A-B.

The control compartment 405 may contain electronic components as described herein. In the embodiment of FIGS. 102-108, the control compartment 405 includes at least a PCB 441 including electrical and control components for powering and controlling components of the system 400. It is understood that the PCB 441, in combination with the PCB 439 in the apparatus 2, may comprise the controller in this embodiment. The components of the control compartment 405 (including the PCB 441) are connected to the main power supply 401, e.g., by a power cord connected to an outlet as shown in FIG. 88, and deliver power and control instructions to the apparatus 2 via one or more electrical conduits 465 running through the umbilical 420. The cabinet 404 has a power input 442 extending through one of the outer walls and into the control compartment 405 to permit connection with the main power supply 401, as well as a main electrical connector 443 that forms a consolidated connection for any power and/or control output cables that extend to the apparatus 2. The main electrical connector 443 may include a quick release connector or other detachable connection as described herein, and is also sealed against water ingress, e.g., by a grommet.

The control compartment 405 may be provided with sealing that is resistant to water or other liquids, to protect from liquid ingress from the plumbing compartment 415 and the outside environment, as well as electromagnetic shielding. The control cabinet 404 of FIGS. 102-108 has an internal wall 444 separating the control compartment 405 from the plumbing compartment 415 that is sealed against liquid ingress, as well as a removable outer panel 445 providing access to an opening 447 to the control compartment 405 from outside the control cabinet 404. The internal wall 444 has a cable port 446 extending therethrough, to permit electronic connections (e.g., power, control, sensor) between the components of the control compartment 405 (e.g., PCB 441) and the components of the plumbing compartment 415 (e.g., the pump 39 and various sensors and valves described herein). The outer panel 445 is removably connected to the control cabinet 404 by a plurality of fasteners in FIGS. 102-108, and a water sealing gasket 448 and an electromagnetic shielding gasket 449 are positioned around the opening 447. Electronic components including at least the PCB 441 are mounted directly on the outer panel 445 in the embodiment of FIGS. 102-108. Other electronic components, such as the AC-to-DC power supply 452 (which includes a cage that provides electromagnetic shielding and air venting), may be mounted on a different wall within the control compartment 405. The outer panel 445 also includes one or more alignment pins 450 that extend through one or more alignment apertures 451 around the opening 447 to ensure the proper alignment of the outer panel 445 relative to the control cabinet 404. The configuration of the control compartment 405 in the embodiment of FIGS. 102-108 provides numerous benefits, including water and electromagnetic shielding, ease of access for components within the control compartment 405, and extreme compactness of design, greatly reducing the size of the entire control cabinet 404. Components such as LEDs 453 and a power button 454 are accessible externally by passing through the walls of the control compartment 405 as well.

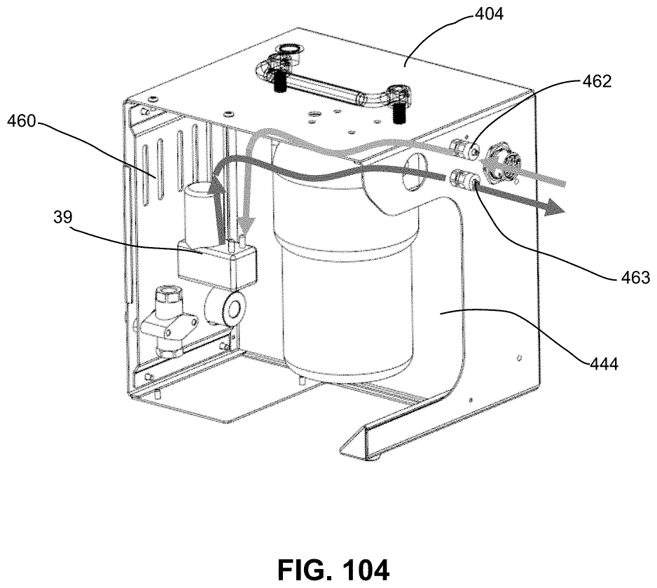

The plumbing compartment 415 generally includes components to control the liquid flow to the apparatus 2 and the condition of the liquid that reaches the apparatus 2. The plumbing compartment 415 in FIGS. 102-108 includes a pump 39 for the exhaust 49 and a filter 410 (e.g., a micropore filter) for outgassing or other filtering, as well as associated components. The liquid may enter the system via the liquid supply 417, which is a main water supply in FIGS. 102-108 but may be a different source in another embodiment, such as another pressurized liquid source. FIG. 108 schematically illustrates the flow of water into and through the plumbing compartment 415 to the filter 410. The liquid supply 417 connects to an inlet 455, and the liquid sequentially flows through a non-return valve (i.e., check valve) 456 and a mechanical flow shut off valve 416 (e.g., a ball valve), then through a solenoid valve 414 that controls the flow of liquid through the cabinet 404 (i.e., a flow restrictor) and a temperature sensor 413. The liquid then flows into and through the filter 410 and then out through the liquid supply conduit 20, which exits the control cabinet 404 through an aperture 457 in the side wall. FIG. 104 schematically depicts the flow of liquid between the apparatus 2 and the pump 39, through the control cabinet 404. The output conduit 458 of the exhaust 49 extends into an inlet port 462 and to the pump 39, then out through an outlet port 463 and through the return conduit 459 of the exhaust 49, which leads to the outlet 54. FIG. 88 illustrates an exhaust stream 54A emitted from the outlet 54 adjacent to and/or into the stream 7.

The control cabinet 404 in FIGS. 102-108 is also provided with a removable internal panel 460 on which is mounted multiple components of the plumbing compartment 415, including at least the non-return valve 456, the shut off valve 416, the solenoid valve 414, the temperature sensor 413, and the pump 39. The removable internal panel 460 is removably connected to one of the inner walls of the control cabinet 404. The control cabinet 404 also has removable internal partitions 461 enclosing the portion of the plumbing compartment 415 that includes the components connected to the removable internal panel 460. This configuration also provides compactness and protects the internal components while allowing access if desired, and also leaves the filter 410 more easily accessible from the exterior of the control cabinet 404 for changing, maintenance, etc.

The configuration of the control cabinet 404 in FIGS. 102-108 may be configured to provide improved heat conduction away from the internal components of the control cabinet 404, particularly the electronic components in the control compartment 405. For example, at least some or all of the panels forming the walls of the control cabinet 404, including the exterior walls, the internal wall 444, the outer panel 445, and/or the internal partitions 461, are made from metal, which effectively conducts heat away from the heat-producing components of the control cabinet 404. In another embodiment, the control cabinet 404 may be configured to even more effectively conduct heat away from the control compartment 405, including using the internal liquid conduits for heat conduction. For example, one or more internal liquid conduits may be run along the side of the internal wall 444 opposite the control compartment 405, to absorb heat that is conducted through the internal wall 444. As another example, one or more walls (e.g., the internal wall 444) may be provided with fins (not shown) or other structures to conduct heat away from the respective wall(s), and such fins may be in contact with one or more internal liquid conduits for further removal of heat. It is noted that the use of metal panels may additionally improve electromagnetic shielding.

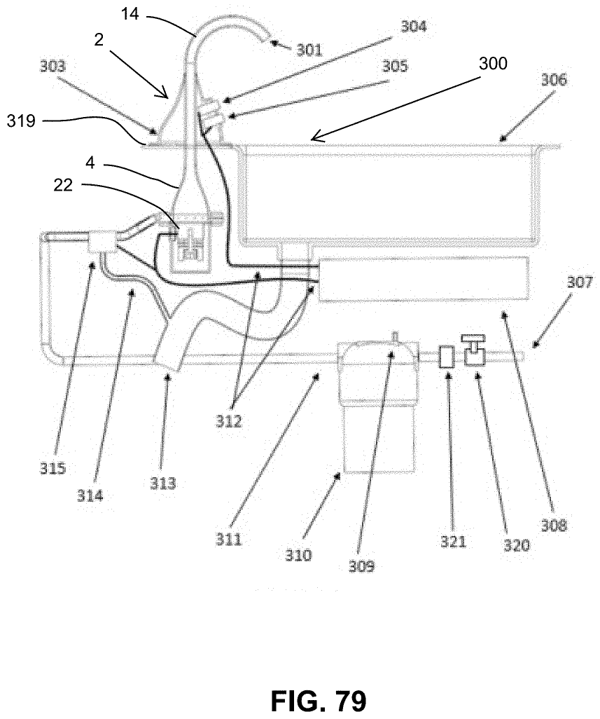

In another embodiment, the apparatus 2 may be integrated into a faucet 301 for use with a sink or basin, as part of system 300 provided as a fixture or a mobile unit for cleaning or other treatment as described herein. FIGS. 79-86 illustrate embodiments of such a system 300 including a faucet 301. Such a system 300 may be provided with some features described herein with respect to FIGS. 71-73. A system 300 provided with such a faucet 301 may have many uses in many different settings, including in the home, public, private, or institutional washrooms, an industrial (manufacturing and/or processing) setting, a laboratory setting, a healthcare setting, an agricultural setting, a workshop or hobby setting, a decontamination setting, etc. For example, the system 300 may be used for personal hygiene (especially hand washing), for washing components, materials, food (e.g., fresh produce), inert and chemically-significant and biologically-significant items and similar items in various settings, for treatment of wounds or other tissue, for sterilization, etc. Such a faucet 301 may be used to provide washing, or pre-washing (e.g., to remove clumps of contaminant, for example from structured surfaces, cracks, and crevices) prior to the main wash, so that the treatments used in the main wash (e.g., chemicals, enzymes, heat) can penetrate to all the contaminants. A pre-wash enhances the ability of the main wash to fully wash the target, and not to produce only a limited wash because matter within the center of larger clumps or agglomerations is protected from the heat and chemicals of the main wash by the outer layers of such clumps or agglomerations. The apparatus 2 may also be provided as part of a system configured as a different type of fixture or mobile unit, including a toilet.

A system 300 using a faucet 301 as described herein may be used for routine washing of items and/or for washing of items in crisis and/or shortage conditions. For example, with respect to a pandemic situation, the system 300 may be used for washing ventilator parts, personal protective equipment, skin, hands, patients, ward rooms, trolleys and gurneys, food and food delivery apparatuses, toilets, etc. The ability of the apparatus 2 to clean with limited or no chemical additives, and without heating, increases effectiveness when other cleaning and decontamination methods that rely on chemical additives (e.g. detergent, bleach, etc.) and/or heating may be compromised by failures in the supply line (e.g., in a pandemic situation) or when the delay to heat liquid before cleaning may cause undesired effects. This may apply to cleaning in many sectors, including healthcare, transport, food production and retail, water treatment, sewage treatment, electricity supply, and other sectors.

An apparatus 2 with a nozzle 14 having a gooseneck or curved configuration (see, e.g., FIGS. 33 and 35) may be advantageous for the use in a faucet 301 such as illustrated in FIGS. 79-86. FIGS. 74-78 illustrate another embodiment of an apparatus 2 that uses a nozzle 14 having a gooseneck configuration, which is adapted for use in a faucet 301 as illustrated in FIGS. 79-86 but may be used in other applications as well. Components and features of the apparatus 2 illustrated in FIGS. 74-78 that have been previously described herein with respect to other embodiments are referenced with the same reference numbers previously used herein. Such components and features that have been previously described may not be described again in detail for the sake of brevity. It is noted that the embodiment shown in FIG. 78 includes a nozzle 14 with a slightly different degree of curvature than the embodiment shown in FIGS. 74-77.

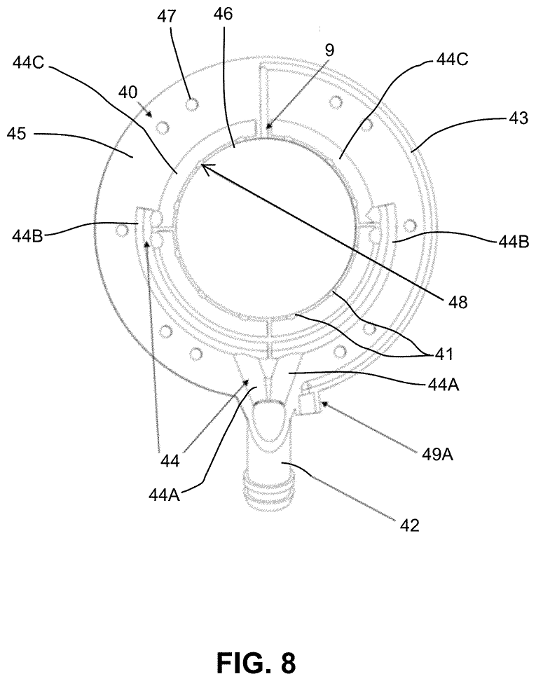

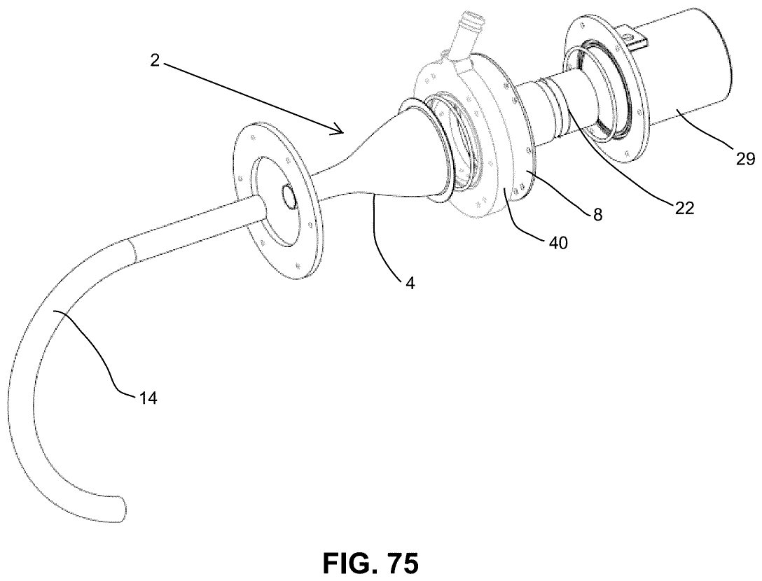

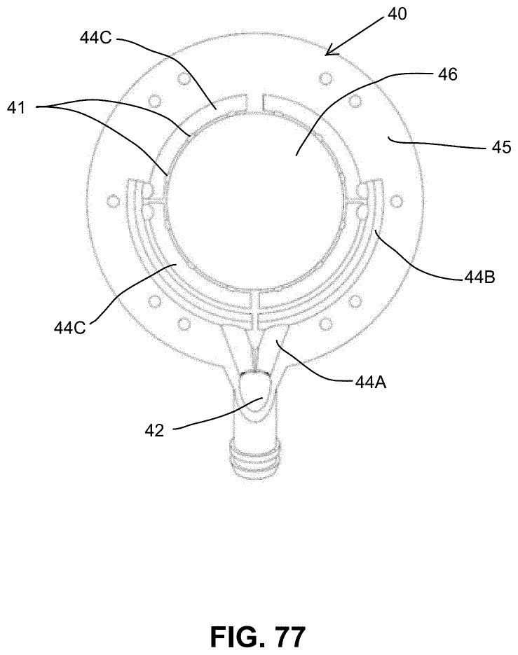

As shown in FIGS. 74-75, the apparatus 2 in this embodiment is provided with a conical body 4 having a nozzle 14, with a manifold 40 for introducing liquid into the chamber 6, a rear wall 8, and a transducer 22 configured to emit acoustic energy at or near the rear wall 8, as similarly described herein with respect to other embodiments. The manifold 40 is shown in greater detail in FIG. 77 and includes many of the components of the manifold 40 shown in FIG. 8 and described herein. For example, the manifold 40 in FIG. 77 includes, among others, a manifold inlet 42 is connected to the liquid supply conduit 20 and one or more internal conduits 44 that extend from the manifold inlet 42 to a plurality of ports 41 distributed around a central cavity 46, such that the ports 41 act as inlets 18 to introduce the liquid into the chamber 6. The manifold 40 in FIG. 77 does not include a vent 9 or an exhaust 49, because the upward-facing configuration of the body 4 avoids the need for venting, as described herein. Similarly, the apparatus 2 in FIGS. 74-78 does not include a choke 60, although a vent 9 and/or a choke 60 may be used in another embodiment.

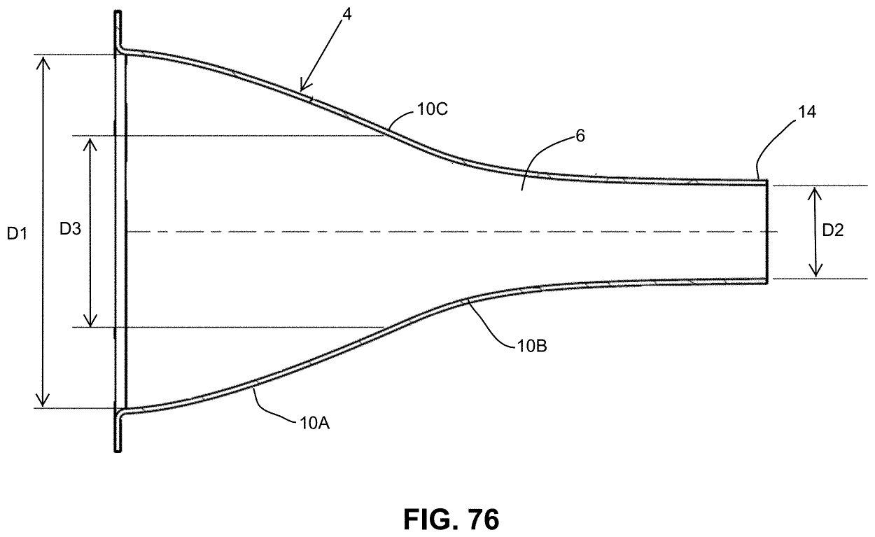

The body 4 of the embodiment in FIGS. 74-78 is illustrated in greater detail in FIG. 76. The body 4 in FIG. 76 has a length L of 95 mm, a diameter D1 at the base 11 of 51.4 mm, a diameter D2 at the nozzle 14 of 13.6 mm, and a wall thickness of 0.7 mm, and the inflection point 10C is located 40 mm from the base 11 of the body 4, with a diameter D3 at the inflection point 10C of 27.8 mm. The body 4 also has an internal chamber 6 volume V of 70.5 mL, and the rear wall 8 has a diameter of 90 mm, with an area A1 having a diameter of 50 mm and a surface area of 1,963 mm² being exposed to the chamber 6. In other embodiments, the body 4 may have a structural configuration with dimensions that are within +/−10% of these values or +/−5% of these values. The body 4 may be formed of a material that enables transmission of acoustic energy by mode coupling as described herein, such as a metal material, or of another material designed for different acoustic properties as described herein. In one embodiment, the body 4 is made from brass. The nozzle 14 may be formed of the same material as the body 4 or a material with similar acoustic modes, in order to achieve transmission of the acoustic energy (assisted by the establishment of propagating acoustic modes that are coupled between the solid and the liquid, as discussed herein) from the body 4 to the nozzle 14. In one embodiment, the body 4 is made from copper and separately connected to the body 4, such as by welding, and in another embodiment, the body 4 is made from brass and either integrally formed with or separately connected to the body 4. As discussed herein, physical breaks such as air gaps, poor welds, use of flanges, gaskets, etc., between the nozzle 14 and the body 4 will reduce the efficiency by which coupled modes propagate.

FIGS. 79-82 illustrate an embodiment of a system 300 that uses an apparatus 2 as described herein and shown in FIGS. 74-78 (with a nozzle 14 as shown in FIG. 78) including a faucet 301 configured to dispense the liquid flowing from the nozzle 14. FIG. 79 illustrates the overall system 300, with a plurality of different components and features, and it is understood that the system 300 may include some or all of these features and/or additional features (including other features described herein) in other embodiments. The faucet 301 in this embodiment is configured so the nozzle 14 empties the liquid into a sink or basin 306 that has a drain 313, which can be used for cleaning or treating one or more targets as described herein. The drain 313 can be connected to a main drain line or to a separate container (e.g., for disposal or later analysis, or as part of a recirculation system 423 as described herein). The faucet 301 may include a casing 303 (e.g., of cast metal) to cover and/or support components of the faucet 301, including at least a portion of the nozzle 14, as well as electronic components. The system 300 in FIG. 79 is configured for connection to a liquid supply 307 in the form of a main water supply (water main), and the water first passes through a regulator 320 to a flow controller 321, which could be fixed or user-controlled as desired. An outgas sing device 310 (such as a micropore filter) may be provided to remove unwanted bubbles and/or excess gas from the liquid. The outgassing device 310 may have a pressure relief valve 309 that would remove excess air in the water line upon installation and maintenance of operation. The outgassing device 310 is connected to a control solenoid 315 by piping 311. The solenoid 315 controls the flow of water into the chamber 6 and out through the nozzle 14. In the embodiment of FIG. 79, the solenoid 315 is provided as a three-way solenoid that also controls the drainage of excess water in the system when not in use. Multiple solenoids may be used for this purpose in another embodiment. The solenoid 315 in FIG. 79 is connected to the main drain 313 via an exhaust tube 314.

The system 300 may include a control box 308 that includes various components for operation of the apparatus 2 (including the solenoid 315), including a power amplifier and some or all components of the controller 19, which are connected to the apparatus 2 (e.g., the transducer 22 and the solenoid 315) by cabling 312. The control box 308 may be provided as part of an integrated fixture or mobile unit along with other components of the system 300 in one embodiment, or may be separately provided. The control box 308 may be located in a sink cabinet below the sink 306 in one embodiment (with appropriate sealing to resist water ingress), or may be located higher than and/or remote from the sink 306 to avoid the risk of contact with the liquid. Additionally, one control box 308 may be configured to power and/or control multiple apparatuses 2 in another embodiment. Further, the components of the control box 308 may instead be provided separately, with no consolidated control box. It is understood that the system 300 may include a control cabinet 404 as shown in FIGS. 71-73B, or that a fixture or mobile unit may be configured similarly to the control cabinet 404 as described herein. For example, the control box 308 may be provided in a system control compartment 405 (and potentially a power amplifier compartment 425) that is/are separate from a water conditioning compartment 415.

The apparatus 2 may be automatically activated in one embodiment, such as by use of a proximity sensor 304 disposed in the casing 303, which is configured to sense the presence of a user or object in position to use the faucet 301. The proximity sensor 304 may transmit proximity information to the control box 308 (e.g., to the controller 19), which may then activate the water flow via the solenoid 315 and activate the transducer 22 at appropriate times according to internal logic. The apparatus 2 may also include a mechanism for user feedback, such as an LED panel 305 disposed in the casing 303, which may receive information for display from the control box 308 (e.g., from the controller 19).

The use of a proximity sensor 304 ensures that the item being washed or treated (such as hands, components, fresh produce, etc.) is at the correct range from the nozzle 14 to ensure good cleaning. Other options to achieve appropriate positioning for the target to be cleaned include use of a rangefinder (e.g., infra-red or in-air ultrasonic) mounted in an appropriate position, e.g., on the nozzle 14 or the casing 303, or an in-liquid ultrasonic pulse transmitted down the liquid stream 7. Such techniques could be used to provide feedback to adjust the off-time between the pulses from transducer 22, so that the bubble bolus has the extra time needed to travel to further ranges down the stream 7, which adjustment may be done by the controller 19 using internal logic. In a further embodiment, the correct positioning of the target to be cleaned could be determined using crossed light beans or lasers producing visible spots that give the user real-time feedback of when the target is correctly positioned, to enable the user to accurately reposition the target as necessary.

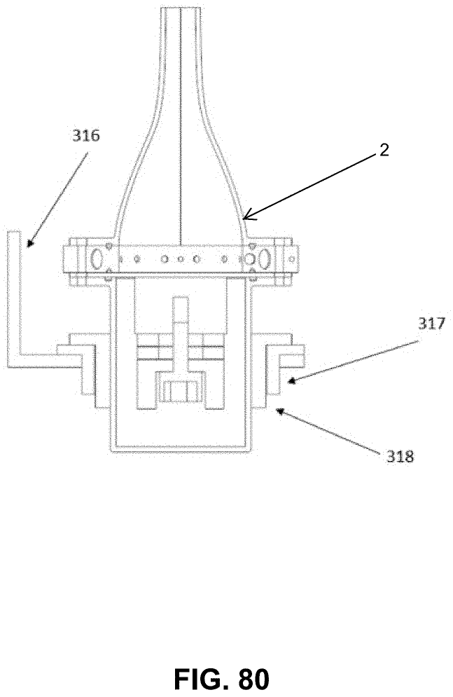

In one embodiment, the apparatus 2 and may be rotatably mounted, to provide a more adaptable faucet 301 configuration. A bearing assembly may be used to achieve at least some degree of rotational movement. FIG. 80 shows one configuration using bearings or bushings 317 positioned between external and internal supporting structures 316, 318, to allow rotation of the internal supporting structure 318 and the apparatus 2 with respect to the external supporting structure 316. The sensors determining the correct positioning of the target to be cleaned, such as the proximity sensor 304, would need to be mounted to rotate with the apparatus 2 in order to continue effective operation. In one embodiment, the casing 303 supporting the proximity sensor 304 (and potentially other sensors) could be fixedly connected to the nozzle 14, such that the casing 303 and any connected sensors would rotate along with the nozzle 14.

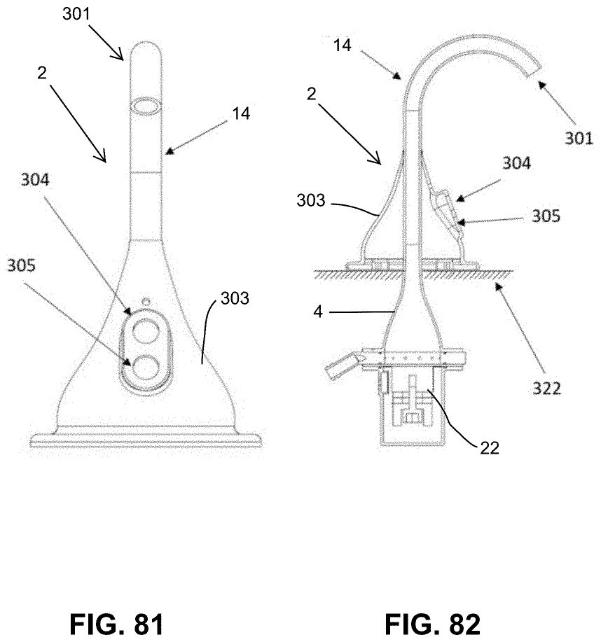

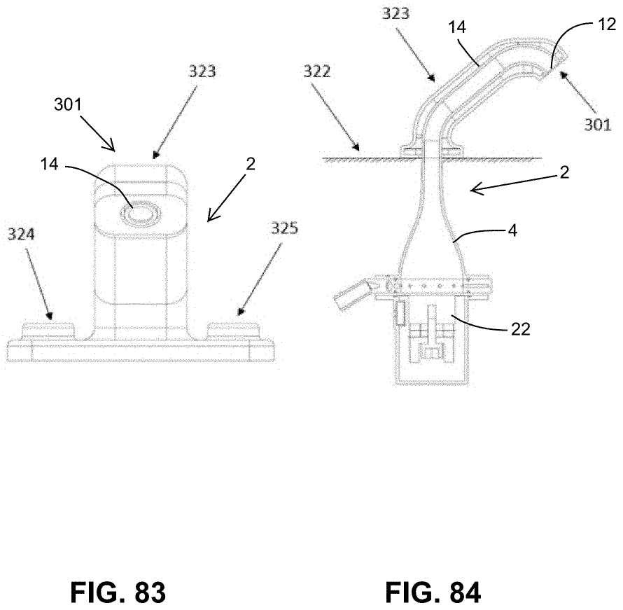

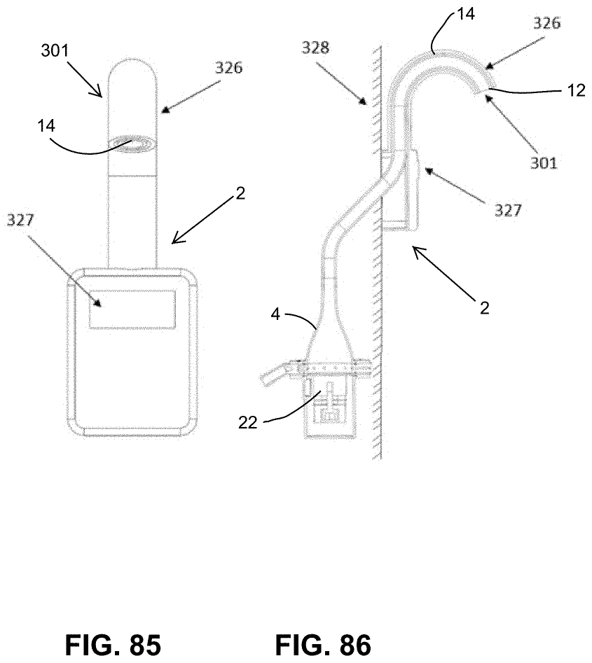

FIGS. 81-86 illustrate various embodiments for mounting a faucet 301 that includes the apparatus 2, all of which use a nozzle 14 with a gooseneck or other curved configuration. FIGS. 81-82 illustrate a faucet 301 configured similarly to the faucet 301 in FIG. 79, which uses a gooseneck nozzle 14 that is partially exposed. In this embodiment, a bulb-shaped casing 303 covers a lower portion of the nozzle 14 and the end of the body 4, and the casing 303 is mounted on a horizontal supporting surface 319 surrounding at least part of the sink 306. A proximity sensor 304 and an LED panel 305 are mounted on the casing 303 in this embodiment as well. FIGS. 83-84 illustrate a faucet 301 that uses a faucet mount 323 that is mounted on a horizontal supporting surface 319 and completely surrounds the portions of the nozzle 14 above the supporting surface 319 other than the orifice 12. The nozzle 14 is hidden from view in such an embodiment. Both the faucet mount 323 and the nozzle 14 in this embodiment have two curved sections 322. The faucet 301 in FIGS. 83-84 also includes manual activation buttons 324, 325 mounted on the faucet mount 323, such as button switches or capacitive sensors. FIGS. 85-86 illustrate a faucet 301 in which the apparatus 2 is partially mounted behind a vertical wall 328, such as within a structural building wall or within a vertical portion of a fixture or a mobile unit. In this configuration, the transducer 22 and the body 4 are mounted behind the wall 328, and the nozzle 14 includes a three-curved configuration that extends through the wall 328 to expose the nozzle 14 on the opposite side of the wall, without curving sharply enough to interfere with propagation of the acoustic energy. The faucet 301 in FIGS. 85-86 includes a faucet mount 326 mounted on the vertical wall 328 that completely surrounds the portions of the nozzle 14 on the outside of the wall 328 other than the orifice 12. The faucet 301 in FIGS. 85-86 also includes one or more sensors 327, such as infrared or ultrasonic sensors, which may function as proximity and/or position sensors as described herein. The sensor(s) 327 in this embodiment are mounted on the faucet mount 326.

It is understood that casing components, such as the casing 303 and the faucet mounts 323, 326 described herein, may be in the form of metal castings that provide structural reinforcement and strong aesthetics.

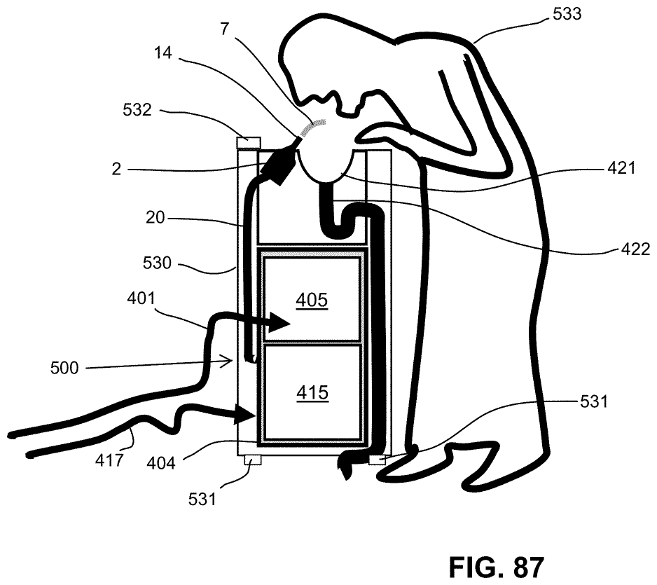

FIG. 87 illustrates another embodiment of a system 500 that uses an apparatus 2 as described herein and includes features and aspects of the systems 300, 400 of FIGS. 71-86, provided in the form of a water dispenser for dispensing water (or other liquid) for drinking and/or oral treatment, such as a drinking fountain. The components of the system 500 may be installed in an existing drinking fountain in one embodiment. The system 500 may be integrated into a fixture 530 that may be provided with feet 531 for resting on the ground as shown in FIG. 87, or may be installed on building structure in another embodiment, e.g., as a wall mounted or floor mounted unit. The fixture 530 may be provided with wheels (including rollers, casters, etc.) to provide a mobile unit in a further embodiment. Components already described herein with respect to the apparatus 2 or the systems 300, 400, may not be described again in detail with respect to FIG. 87 for the sake of brevity.

The fixture 530 of FIG. 87 includes a control cabinet 404 arranged similarly to the control cabinet 404 of FIG. 73A, with a system control compartment 405 and a water conditioning compartment 415, and may also include a power amplifier compartment (not shown) as described with respect to FIG. 73B. The control cabinet 404, the system control compartment 405, and the water conditioning compartment 415 may include any or all of the components discussed herein or illustrated in FIGS. 71-73B. For example, the control cabinet 404 is connected to a main power supply 401, which may supply power to the apparatus 2 via a high voltage power cable (not shown) connected to the system control compartment 405. The components of the system control compartment 405 (e.g., a controller) may also send control signals to the apparatus 2 via a low voltage communication cable (not shown). The water conditioning compartment 415 is connected to a main water supply 417 and delivers liquid to the apparatus 2 through a liquid supply conduit 20. The control cabinet 404 and/or the components or compartments 405, 415 therein, may be electromagnetically shielded as discussed herein.

The apparatus 2 in FIG. 87 is positioned so the nozzle 14 directs the stream 7 into a sink or basin 421 with a drain 422 that removes the liquid after use. The stream 7 is positioned and oriented to permit a user 533 to intake the stream orally, as illustrated in FIG. 87, such as for drinking or mouth washing. Various orientations of the apparatus 2 and/or configurations of the nozzle 14 as described herein may be used in the apparatus 2 of FIG. 87, including nozzles 14 as shown in FIGS. 74-86 that may direct the stream horizontally and/or downward with an upwardly-oriented body 4. It is understood that the apparatus 2 may include components described herein with respect to other embodiments, including at least a body 4 with a chamber 6 in communication with the nozzle 14, and a transducer 22 configured for emitting acoustic energy into the liquid. Depending on the orientation of the body 4 and the nozzle 14, a vent and exhaust (not shown) may also be included. Accordingly, the apparatus 2 may include a manifold with a simplified structure that does not include a vent 9. The nozzle 14 illustrated in FIG. 87 is a straight nozzle 14 that directs the stream 7 in a direction that is within 45° of vertical, such that a choke and/or a vent/exhaust is not needed. It is understood that in other embodiments, a different angle may be used, which may or may not require the use of choke and/or a vent/exhaust. Gas and bubbles can be removed from the chamber 6 by buoyancy in this configuration, as discussed herein. The drain 422 may direct the liquid to a waste water disposal location. The system 500 and fixture 530 of FIG. 87 may also include a sensor (e.g., infrared) to detect the presence of a user and automatically activate the water flow and the transducer 22, such as via communication with a controller. In another embodiment, the fixture 530 may include a pedal or button for manual activation.

The system 500 in FIG. 87 could be used to provide oral freshness and hygiene, such as reducing bacteria, odor, etc., by providing a stream 7 of water with acoustically activated bubbles to clean or provide other treatment to the mouth of the user 533. The system 500 and fixture 530 may be provided in many different uses and applications. For example, the system 500 and fixture 530 may be deployed in a workplace for use by workers or in an airport or railway lounge for use by travelers (particularly those on long stop-overs), as an alternative to using brushes or mouthwashes in a restroom that may also include toilets. As another example, the system 500 and fixture 530 may be deployed in waiting rooms (e.g. for dentists, doctors, government agencies, car rental services, etc.), where use of the fixture 530 will improve oral hygiene and may be conducive to regular use because people often wait in such locations for long periods of time while not otherwise occupied. As a further example, the system 500 and fixture 530 may be deployed in various locations in rural and/or low income areas, where access to personal oral care may be limited. The system 500 may be installed in any location that includes drinking water facilities, e.g., a drinking fountain, and the system 500 may be installed within a drinking water fixture 530 such as a water fountain. The fixture 530 may be configured to provide water from the nozzle 14 with acoustic energy and bubbles for cleaning or other treatment, or to provide water from the nozzle 14 without acoustic energy and bubbles for drinking purposes. In another embodiment, the fixture 530 may include a separate nozzle 14 (and potentially a second basin 421) for use with the apparatus 2 so that drinking water and oral cleaning or other treatment is not done with the same nozzle 14 and/or within the same basin 421. In a further embodiment, the fixture 530 may be provided with separate hot and cold water supply as described above with respect to FIGS. 71-72. The fixture 530 may be provided with an apparatus 2 as described herein to dispense cold water and a normal faucet or other water outlet for dispensing hot water, as also discussed herein. The fixture 530 may include any of the components discussed herein with respect to FIGS. 71-72, including with respect to the hot and cold water embodiment discussed herein.

Use of the system 500 provides the convenience of oral cleaning and hygiene improvement without the necessity of carrying or purchasing consumables such as a toothbrush, toothpaste, or mouthwash. The system 500 can provide cleaning using water alone and may be configured to deliver water with various additives, such as fluoride, mouthwash, etc. In one embodiment, the fixture 530 may include an interface 532 for user interaction, e.g., a touchscreen and/or a wireless interface for communication with a user's mobile device, which permits a user to select one or more additives to be included in the stream 7. Such additives may be provided as a paid service in one embodiment. The interface 532 may further be configured to permit user selection to deliver water only, without acoustic energy.

The system 500 could further be incorporated in a drinking apparatus for use by livestock or other animals, to provide oral cleaning or other treatment while the animal drinks. In particular, some species (e.g., apes, some rodents) can drink from streams emitted by nozzles, facilitating use of the system 500. The routine introduction of drinking streams that periodically contain acoustic energy could reduce the oral healthcare bill for a large institution containing high-value animals (e.g., a zoo, an equestrian stable, a cattle lot) considerably.

The apparatus and method described herein produces numerous technical advantages over existing cleaning technologies, in particular, over ultrasound baths in which the target is immersed in the cleaning liquid. One such advantage is that the liquid stream can be directed to a particular location as desired, and can be directed onto an object of any size, where a cleaning bath can only be used to clean within the bath itself, which is unable to accept any object larger than the dimensions of the bath. This is enabled by the ability of the apparatus to propagate resonant bubbles and acoustic energy sufficient for energizing those resonant bubbles down a stream to a distant target. Another such advantage is that the liquid is not retained for a subsequent cleaning, reducing the risk of cross-contamination. A further advantage is the potential for the apparatus to be portable. Yet another advantage is the capability of the apparatus to be used along with other similar apparatuses in a large-scale cleaning system. Still other advantages are recognized by those skilled in the art.

The apparatus and method described herein may be used as an effective cleaning system to intervene at an early stage of a potential pandemic, by reducing the ability of the virus to reside on touch surfaces, and so infect one person as their hands carry virus to their face, directly or indirectly, from touching a surface (e.g., counter, keypad etc.) that was touched previously by an infected person. This can be a powerful intervention, because this infection prevention technology is generally effective against microbes (bacteria, viruses, fungi, parasites, etc.) and the host materials in which they can reside (grease, biofilms, secretions, residues, etc.), and it is not specific to any individual pathogen. As a result, such an intervention can be introduced without the delay required to devise, test and distribute vaccines and drug treatments, sequence RNA or DNA as relevant, etc. An appropriate faucet containing an apparatus as described herein could be embedded in routine use in, for example, the home, healthcare facilities, hospitality, catering, aircraft, cruise lines, etc., prior to any pandemic. The benefits from the technology could therefore be effective from the earliest days before an infection becomes a pandemic, potentially reducing the possibility of pandemic development. The apparatus could be configured to resemble a traditional faucet externally and could be implemented as such, by being integrated into a traditional sink or bath fixture. This usage would permit the apparatus to be easily adopted without the delay and limitations in take-up required by implementation, training, etc. In this usage, the apparatus could ease the burden on healthcare systems by reducing infections of all types within and outside of pandemic periods. The apparatus may also be used to more quickly and effectively clean ventilator components, personal protective equipment, and other critical items for healthcare for re-use. In fact, the apparatus may permit effective cleaning in crisis situations where supply lines are disrupted, affecting the availability of detergent, soap, bleach, and other cleaning agents.

Various embodiments of cleaning apparatuses, as well as systems and methods incorporating the same, have been described herein, which include various components and features. In other embodiments, the apparatus, system, and method may be provided with any combination of such components and features. It is also understood that in other embodiments, the various devices, components, and features of the apparatus and system described herein may be constructed with similar structural and functional elements having different configurations, including different ornamental appearances.

Several alternative embodiments and examples have been described and illustrated herein. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the spirit or central characteristics thereof. The present examples and embodiments, therefore, are to be considered in all respects as illustrative and not restrictive, and the invention is not to be limited to the details given herein. Terms such as “top,” “bottom,” “front,” “back,” “side,” “rear,” “proximal,” “distal,” and the like, as used herein, are intended for illustrative and relative purposes only and do not limit the embodiments in any way. It is understood that an apparatus may be used in a variety of different orientations that may change the positions of the components thereof. For example, a “base” as described herein may not be at the bottom of the apparatus in some use orientations. When used in description of a method or process, the term “providing” (or variations thereof) as used herein means generally making an article available for further actions, and does not imply that the entity “providing” the article manufactured, assembled, or otherwise produced the article. Nothing in this specification should be construed as requiring a specific three dimensional orientation of structures in order to fall within the scope of this invention, unless explicitly specified by the claims. Additionally, the term “plurality,” as used herein, indicates any number greater than one, either disjunctively or conjunctively, as necessary, up to an infinite number. Accordingly, while the specific embodiments have been illustrated and described, numerous modifications come to mind without significantly departing from the spirit of the invention and the scope of protection is only limited by the scope of the accompanying claims.

What is claimed is:

1. An apparatus comprising:
a body defining a chamber configured to contain a liquid and having a nozzle located a front end of the body and configured to discharge a stream of the liquid from the chamber toward a surface;
a rear wall positioned at a rear of the body and at least partially defining the chamber;
an inlet in communication with the chamber and configured for connection to an inlet conduit to introduce an inlet stream of the liquid into the chamber;
an acoustic transducer positioned at the rear wall, the acoustic transducer configured to generate acoustic energy and to introduce the acoustic energy into the liquid contained in the chamber; and
an exhaust system comprising a vent in communication with the chamber and configured to remove a mixture of liquid and gas bubbles from the chamber, an exhaust leading away from the vent, and an outlet in communication with the exhaust and configured to discharge the mixture of liquid and gas bubbles into or adjacent to the stream.

2. The apparatus of claim 1, wherein the body is a conical body comprising a conical wall having a concavely curved portion more proximate to the rear wall and a convexly curved portion between the concavely curved portion and the nozzle, when viewed from inside the chamber, and wherein an inflection point is located between the concavely curved portion and the convexly curved portion.

3. The apparatus of claim 1, wherein the outlet is positioned to discharge the mixture of liquid and gas bubbles into the stream at a location within the nozzle.

4. The apparatus of claim 1, wherein the outlet is positioned to discharge the mixture of liquid and gas bubbles into or adjacent to the stream at a location adjacent to the nozzle.

5. The apparatus of claim 1, wherein the exhaust system further comprises a pump in communication with the exhaust and configured to draw the mixture of liquid and gas bubbles from the vent and to pump the mixture of liquid and gas bubbles to the outlet.

6. The apparatus of claim 1, further comprising a manifold located at the rear of the body and connected to an inlet conduit to receive the inlet stream, the manifold further having a plurality of inlets distributed around the manifold and configured for introducing the inlet stream into the chamber.

7. The apparatus of claim 6, wherein the vent is formed in the manifold.

8. The apparatus of claim 6, wherein the manifold includes a central cavity, and the inlets are distributed around the central cavity and configured to introduce the liquid into the central cavity.

9. The apparatus of claim 1, further comprising a casing having a handle, wherein the body, the rear wall, the transducer, the inlet, the vent, and the outlet are positioned within the casing, and wherein the nozzle is positioned to discharge the stream out of the casing.

10. The apparatus of claim 9, wherein the exhaust comprises an output conduit extending out of the casing and a return conduit extending back into the casing and to the outlet.

11. The apparatus of claim 10, wherein the exhaust system further comprises a pump in communication with the exhaust and configured to draw the mixture of liquid and gas bubbles from the vent and to pump the mixture of liquid and gas bubbles to the outlet, and wherein the pump is located outside the casing.

12. An apparatus comprising:
a conical body defining a chamber configured to contain a liquid and having a nozzle located a front end of the conical body and configured to discharge a stream of the liquid from the chamber toward a surface;
a rear wall positioned at a rear of the conical body and at least partially defining the chamber;
a manifold located at the rear of the conical body and engaged with the conical body and the rear wall, wherein the manifold is connected to a liquid supply conduit to receive an inlet stream of the liquid and has a plurality of inlets in communication with the chamber, the inlets being distributed around the manifold and configured for introducing the inlet stream into the chamber;
an acoustic transducer positioned at the rear wall, the acoustic transducer configured to generate acoustic energy and to introduce the acoustic energy into the liquid contained in the chamber; and
an exhaust system comprising:
a vent formed in the manifold in communication with the chamber and configured to remove a mixture of liquid and gas bubbles from the chamber;
an output conduit connected to the manifold and leading away from the vent;
a pump in communication with the output conduit and configured to draw the mixture of liquid and gas bubbles from the vent via the output conduit;
a return conduit in communication with the pump; and
an outlet in communication with the return conduit and configured to discharge the mixture of liquid and gas bubbles into or adjacent to the stream at a location adjacent to the nozzle,
wherein the pump is further configured to pump the mixture of liquid and gas bubbles to the outlet via the return conduit.

13. The apparatus of claim 12, wherein the manifold includes a central cavity, and the inlets are distributed around the central cavity and configured to introduce the liquid into the central cavity.

14. The apparatus of claim 12, further comprising a casing having a handle, wherein the conical body, the rear wall, the transducer, the manifold, the vent, and the outlet are positioned within the casing, and wherein the nozzle is positioned to discharge the stream out of the casing.

15. The apparatus of claim 14, wherein the pump is located outside the casing, the output conduit extends out of the casing to the pump, and the return conduit extends from the pump back into the casing and to the outlet.

16. A system comprising the apparatus of claim 12 and a control cabinet located remote from the apparatus, the system further comprising an electrical conduit extending from the control cabinet to the apparatus, wherein the liquid supply conduit extends from the control cabinet to the manifold to supply the inlet stream of the liquid to the manifold, and the electrical conduit is connected to the transducer.

17. The system of claim 16, wherein the control cabinet comprises an outgasser in communication with the liquid supply conduit and configured to remove gas from the inlet stream.

18. The system of claim 17, wherein the outgasser comprises a micropore filter.

19. The system of claim 16, wherein the pump is located in the control cabinet, and wherein the output conduit and the return conduit extend to and from the control cabinet, respectively.

20. A system comprising:
a liquid supply conduit configured to supply an inlet stream of liquid;
a control cabinet comprising an outgasser in communication with the liquid supply conduit and configured to remove gas from the inlet stream, and a pump; and
an apparatus comprising:
a conical body defining a chamber configured to contain a liquid and having a nozzle located a front end of the conical body and configured to discharge a stream of the liquid from the chamber toward a surface;
a rear wall positioned at a rear of the conical body and at least partially defining the chamber;
a manifold located at the rear of the conical body and engaged with the conical body and the rear wall, wherein the manifold is connected to the liquid supply conduit to receive the inlet stream of the liquid and has a plurality of inlets in communication with the chamber, the inlets being distributed around the manifold and configured for introducing the inlet stream into the chamber;
an acoustic transducer positioned at the rear wall, the acoustic transducer configured to generate acoustic energy and to introduce the acoustic energy into the liquid contained in the chamber; and
an exhaust system comprising:
a vent formed in the manifold in communication with the chamber and configured to remove a mixture of liquid and gas bubbles from the chamber;
an output conduit connected to the manifold and leading away from the vent to the pump;
a return conduit in communication with the pump; and
an outlet in communication with the return conduit,
wherein the pump is configured to draw the mixture of liquid and gas bubbles from the vent via the output conduit and to pump the mixture of liquid and gas bubbles to the outlet via the return conduit, and wherein the outlet is configured to discharge the mixture of liquid and gas bubbles into or adjacent to the stream at a location adjacent to the nozzle.
